SEQUENCE LISTING (19 sequences)

SEQ ID NO: 1 - DNA, 301 bp, Cucurbita moschata
  source 1..301: mol_type = genomic DNA
ttataattagtattagaattaggaatcgaatggtgtgccaacaaggacactgacccatacctaatacacgacgagtaagaacttatatgtattttttttaattgaaattgattgataaggctaatggaatgaaaagaatgaataaaattgrttaaagaggtatataaagacccacatatcatcatgattgttgcccatttactcaatcttccgttctcataattattcctaataataattaaccaaactaaatgtctaacataattttattttcaaaatttagatatttttgtcactaata

SEQ ID NO: 2 - DNA, 301 bp, Cucurbita moschata
  source 1..301: mol_type = genomic DNA
gtagccattgtcaaatgactcaaattctgtgttttgctgtgactcaaagcatcaaaaataatattatttacattttaatttaatttaattttttttataaagaaaaaagtggatacattatttcccttatgtcctaagcatatggcattastccctaaacaggattagccctaataatgcaattcttgaactcaaattagaaagcatcaatttttaatcccattcgagttttgtagggtagtgaccagattgaggtactttcacgtgttcaacaaactatagcgtaacgttggagtaaatt

SEQ ID NO: 3 - DNA, 301 bp, Cucurbita moschata
  source 1..301: mol_type = genomic DNA
aatttaatttaattttttttataaagaaaaaagtggatacattatttcccttatgtcctaagcatatggcattagtccctaaacaggattagccctaataatgcaattcttgaactcaaattagaaagcatcaatttttaatcccattcgrgttttgtagggtagtgaccagattgaggtactttcacgtgttcaacaaactatagcgtaacgttggagtaaattaaaagtacatgaatgaatcgacactatgaatattattaagaacttcaaacgtaagtatgaagttgttgatgacatt

SEQ ID NO: 4 - DNA, 301 bp, Cucurbita moschata
  source 1..301: mol_type = genomic DNA
gcccgtgctacagcctctcccccgttcgcctcggcatcaataccgcctcggcaaatgccagaattttcaacttagatatgaacaagatatggctattctaaggtcgaatgcgtttcgaggaatacgaaagtgaatctcatatgaacaacamtgttggttccacccctacatcacccaaaaatagtgaaaagaattcaataaatcctaggagtctaaaaaaaaaagtatggaaaatggttacaccgaggaggaatcggcgaaaacttgattaagcatatgatcgtcgtcgttgccgttgggt

SEQ ID NO: 5 - DNA, 301 bp, Cucurbita moschata
  source 1..301: mol_type = genomic DNA
aattaaatttcataaatcgtaatgaaattatacgtagcttcttttaagttatttttagctatgttatatttggaatcgctgtcattcatatactcttttttttactcgggtgtcacatagctatcctcttaaatcacgcgtaataatatcrtattaattagctaattatacatgatatgaggagatttggatttggggacgaatatgggattttctaccttatttggaaatttcataattgattccaatttaatttggaataaatcttaaaccaaatccaaaatataatggatttttcttg

SEQ ID NO: 6 - DNA, 301 bp, Cucurbita moschata; identical to SEQ ID NO: 4
  source 1..301: mol_type = genomic DNA

SEQ ID NO: 7 - DNA, 301 bp, Cucurbita moschata
  source 1..301: mol_type = genomic DNA
ctcatactaatggagattgtgtccctcacttatgtacccatgagcttcctgttaattagccaacgtggaactcaaacaatcctcaacaatcttcacatgggacaaagtacaccatatagcctctcctaaggcctatctacttctttggagyccttgaacaacctttgtttaatcgaagttcaactcctttcgagcccttggagaaaatacaccctctattcaacacgtgactcactaaaacagctcacaactttcaaagaaaaatatttttacaattgtttcttttaaaaagccactcaac

SEQ ID NO: 8 - DNA, 301 bp, Cucurbita moschata
  source 1..301: mol_type = genomic DNA
ttagagaataaataataaaaaattaattataaaataaaataaatatatataattgagttcgttagttatattcaaaattttgttatggttaattcataaaatatctatttattaaaattatttttcaaaaaaaaatttgtaagagcagtgrgagaataaataataaaaaatagtaataaaataaaataaaataaatatactgtatgtataactgtattataagctaattttttttaatcaacagtaaatttgaattaatttgttatcaggggaatctactcactaattcggtccataaaat

SEQ ID NO: 9 - DNA, 301 bp, Cucurbita moschata
  source 1..301: mol_type = genomic DNA
aatatggtctacattttgtcactgctacccatactacctattctattgtgggttcaaatctattaacttgttgttactaactctattaactaatcctcctaaagatgctattgtataggatctagattcatattagagtcatgactttaasttaaccatataaatataatcctcaaatgtcgaataaaaaagttatgaatctagaaggtgtaggcaaaaagtgactcaagtgcgaactcgggtgtactttgttttgagggctctagagaaagaagtcaagactcaattaaagggaggttgt

SEQ ID NO: 10 - DNA, 301 bp, Cucurbita moschata
  source 1..301: mol_type = genomic DNA
caatccgtaacctactagtagaggaagaccccctaaaaataaaagacgtagggccaaccagagaatcaaaagaggatatttttgtgcagaattttaaagatttttctaatatatttaatctgtgtcagaaagattaaactcggaatctttsatcgaatacattatgtttaacttatttaaaaaggttagaggtttaaattaataatgatatttactattttatctactgaattatgctcaaatgggtaaaaaaaataaaaaaacaaacattttccacgaatctttaattactaaagttttt

SEQ ID NO: 11 - DNA, 301 bp, Cucurbita moschata
  source 1..301: mol_type = genomic DNA
accaaaaagtaatgtataataattttctttacagaaatttatctttctttttttccttttccaaaaggttaaattaagataattttttatatttcatagtaaatttaatattcaattttatcgtataaaataaataggtgcaataataggyaattaataataataatcgagatttttttattaaaaaataatgcattatatttaggcaatttttaaaaataaataaatttaacaaagaaaaaaaaaacaggagagaagctgcaattgacaaagctcacgtacatgcaaagtccaatgagga

SEQ ID NO: 12 - DNA, 301 bp, Cucurbita moschata
  source 1..301: mol_type = genomic DNA
tgtaagcatgaacttgtgttggtatgtggagataatatgatcgtatgatataagggatgcaaagacatgtcaaggtcatttagtaggtaacgtggtcatgtcgcaagagacgtaagtaagtgaatgttaggcttattttgtgctagtcgaygaagtgaccatgcggcagaggatggcgtttgtagcaatcaaaactgggtgagagtgtgcatgaaaacaaactgtcaagaattgaggagttgagaatgttaaaagggtttgatttgatggcaaaacgcttccctctcatcctttttccctc

SEQ ID NO: 13 - DNA, 180 bp, Cucurbita moschata
  source 1..180: mol_type = genomic DNA
aaaaaaaaaaaaaaaaaaaaaaaaaaaaagggaaggcagatccaaggaggtggttaatctgtcaatcccatcaatcagtcaaccaaacacgaccaatcatcaatcatcaatcaagattttataaaaagcaaaggcgaaaatacaataatcttgatatgtcatcctcttcctcttcttctt

SEQ ID NO: 14 - DNA, 104 bp, synthetic construct
  source 1..104: mol_type = other DNA
aaaaaaccaaacacgaccaatcatcaatcatcaatcaagattttataaaaagcaaaggcgaaaatacaataatcttgatatgtcatcctcttcctcttcttctt

SEQ ID NO: 15 - DNA, 104 bp, synthetic construct; identical to SEQ ID NO: 14
  source 1..104: mol_type = other DNA

SEQ ID NO: 16 - DNA, 96 bp, synthetic construct
  source 1..96: mol_type = other DNA
aaaaaaaaaaaaaaaaaaaaaaaaaaaaagggaaggcagtcaaccaaacacgaccaatcatcaatcatcaatcaagattttataaaaagcaaaggc

SEQ ID NO: 17 - DNA, 82 bp, synthetic construct
  source 1..82: mol_type = other DNA
aaaaaaaaaaaaaaaaaaaaaaaaaaaaagggaaggcgaccaatcatcaatcatcaatcaagattttataaaaagcaaaggc

SEQ ID NO: 18 - DNA, 65 bp, synthetic construct
  source 1..65: mol_type = other DNA
aaaaaaaaaaaaaaaaaaacgaccaatcatcaatcatcaatcaagattttataaaaagcaaaggc

SEQ ID NO: 19 - DNA, 43 bp, synthetic construct
  source 1..43: mol_type = other DNA
aaaaaaaaaaaaaaaaaaaaaaaaaaaaagggaaggcaaaggc

(19) United States
(12) Patent Application Publication
(10) Pub. No.: US 2025/0113796 A1
(43) Pub. Date: Apr. 10, 2025

(54) CUCURBITA WITH DETERMINATE GROWTH HABIT

(71) Applicant: Vilmorin & Cie, Paris (FR)

(72) Inventors: Florian LEPLAT, Avignon (FR); Jeremie DENONFOUX, La Bohalle (FR); Van TRAN NU THANH, Ho Chi Minh (VN)

(21) Appl. No.: 18/907,400

(22) Filed: Oct. 4, 2024

Related U.S. Application Data
(60) Provisional application No. 63/588,683, filed on Oct. 6, 2023.

(51) Int. Cl. A01H 6/34; A01H 1/00; A01H 5/10

(52) U.S. Cl. CPC A01H 6/348; A01H 1/121; A01H 5/10

(57) ABSTRACT

The disclosure provides novel Cucurbita plants having a determinate growth habit and plants comprising a QTL associated with said determinate growth trait. The disclosure provides a Cucurbita plant having a determinate growth habit and its derivatives. The disclosure also provides molecular markers linked to said QTL. The disclosure further provides methods for producing plants having a genetic determinacy in growth habit and maturity timing, and the plants produced by such methods.

### CROSS-REFERENCE TO RELATED APPLICATIONS

The present application claims the benefit of priority to U.S. Provisional Application No. 63/588,683 filed on Oct. 6, 2023, the contents of which are hereby incorporated in its entirety.

### TECHNICAL FIELD

The disclosure relates to plant breeding, plant physiology and molecular biology. Particularly, the disclosure relates to identification of plants of the genus Cucurbita with a genetic determinacy in plant growth and maturity, methods of developing plants of the genus Cucurbita (e.g., through plant breeding) with the desired growth determinacy; and to the plants of the genus Cucurbita developed by such methods.

### REFERENCE TO AN ELECTRONIC SEQUENCE LISTING

The contents of the electronic sequence listing (VILM_055_01US_SeqList_ST26.xml; Size: 21,711 bytes; and Date of Creation: Oct. 4, 2024) are herein incorporated by reference in its entirety.

### BACKGROUND

Some of the World's most valuable crops, including watermelon, honey melon, cucumber, squash, zucchini and pumpkin, belong to the family Cucurbitaceae. Production of Cucurbitaceae crops in large quantities is important worldwide, since cucurbits are important commercially in many regions, and are increasingly available throughout the year.

The genus Cucurbita is well known for its cultivated species which in English are called by the general names of squashes, pumpkins, marrows and gourds, and in Spanish are commonly known as calabazas and zapallos. Cucurbita species are cultivated worldwide, and are an important food source for much of the world's population. According to FAO data, global production of squash, pumpkins, and gourds was estimated to be 16.2 million tons in 2001 (Taylor and Brant, 2002).

The Cucurbita species such as squash and pumpkins are important horticultural crops worldwide, but there has been relatively little research to systematically describe yield components and improve productivity in this species. Surprisingly, there is a lack of public breeders working with species in the genus Cucurbita for fundamental problems of crop productivity, while most of the breeders have focused on pathological problems. Most cultural research in this genus has dealt primarily with nutrient and moisture responses in relation to fresh weight yields, with little attention to problems associated with the physiology of productivity (Loy, 2004).

Therefore, there is an important need in the art to identify traits associated with semi-determinate or determinate architecture in Cucurbita plants and utilize reliable source of a semi-determinate or determinate growth trait which could then be applied to manage plant size and fruit yield in commercial plants of the genus Cucurbita. To accomplish this goal, the Cucurbita breeder must select and develop Cucurbita plants that have the traits of interest.

### SUMMARY

The present inventors have identified C. moschata plants which display a plant architecture with a semi-determinate or determinate growth habit depending on the heterozygous or homozygous manner of the trait, which can be used for managing plant size and fruit set. The present disclosure provides that Cucurbita plants carrying the genetic determinacy have a limited number of internodes and shorter internodes than indeterminate Cucurbita plants. The present disclosure further provides that compact plants with the semi-determinate or determinate growth habit allow for increased plant density in the field and greenhouse which will benefit breeders and growers. In some embodiments, the claimed semi-determinate or determinate growth habit has been able to be introgressed into Cucurbita plants by introducing C. moschata sequences (i.e. quantitative trait loci (QTLs)) conferring the desired genetic determinacy, thus obtaining Cucurbita plants with semi-determinate or determinate growth habit.

The present disclosure provides that the constrained fruit set obtained from the claimed Cucurbita plants displaying a semi-determinate or determinate growth habit leads to increased uniformity of fruit maturity at harvest. This helps solve the issue of immature fruit being harvested with mature fruit.

The present disclosure provides a plant of Cucurbita variety PUV008, having a determinate growth habit, wherein a representative sample of plant tissue of said variety was deposited under NCIMB No. 43740. In some embodiments, a Cucurbita seed is produced by growing the plant of Cucurbita variety PUV008 deposited under NCIMB No. 43740. In some embodiments, a method of producing a Cucurbita plant, or part thereof, comprises growing the seed produced by grown the plant of Cucurbita variety PUV008. In some embodiments, a Cucurbita plant, or part thereof, has all of the physiological and morphological characteristics of the Cucurbita plant of Cucurbita variety PUV008. In some embodiments, a method of vegetatively propagating the plant of Cucurbita variety PUV008 comprises the steps of: a) collecting tissue or cells capable of being propagated from said plant; b) cultivating said tissue or cells to obtain proliferated shoots or plantlets; and c) rooting said proliferated shoots or plantlets to obtain rooted shoots or rooted plantlets. In some embodiments, a Cucurbita plant is produced by growing the rooted shoots or rooted plantlets described herein.

In some embodiments, a method of determining the genotype of the Cucurbita plant of Cucurbita variety PUV008 comprises obtaining a sample of nucleic acids from said plant and detecting in said nucleic acids a plurality of polymorphisms.

In some embodiments, a method of introducing a mutation into the genome of a PUV008 plant comprises mutagenesis of the plant, or plant part of Cucurbita variety PUV008, wherein said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.

The present disclosure teaches a method of genetically modifying the plant of Cucurbita variety PUV008, wherein said genetic modification is selected from agrobacterium-mediated gene transfer, protoplast transformation, or biolistic transformation. In some embodiments, a plant produced by the method of genetically modifying the plant of Cucurbita variety PUV008 has the determinate growth habit.

In some embodiments, a method of editing the genome of Cucurbita plant PUV008 comprises editing the genome of the plant, or a part of Cucurbita plant PUV008. In some embodiments, said method is selected from the group comprising zinc finger nucleases, transcription activator-like effector nucleases (TALENs), engineered homing endonucleases/meganucleases, and the clustered regularly interspaced short palindromic repeat (CRISPR)-associated protein9 (Cas9) system. In some embodiments, a Cucurbita plant is produced by the method of editing the genome of Cucurbita plant PUV008.

In some embodiments, a method for developing a Cucurbita plant having a determinate growth habit comprises: applying plant breeding techniques to the plant of Cucurbita variety PUV008, having a determinate growth habit, wherein a representative sample of plant tissue of said variety was deposited under NCIMB No. 43740, to produce a plant having a determinate growth habit. In some embodiments, said plant breeding technique is recurrent selection, mass selection, hybridization, open-pollination, backcrossing, or pedigree breeding. In some embodiments, said plant breeding technique is mutation breeding, and wherein said mutation selected is spontaneous or artificially induced. In some embodiments, said plant breeding technique is marker enhanced selection.

The present disclosure provides a method for producing a Cucurbita plant having a determinate growth habit, said method comprising: a) crossing two Cucurbita plants, b) harvesting the resultant Cucurbita seed, c) growing the seed from step b) to produce progeny, and d) selecting resulting progeny for the determinate growth habit; wherein at least one of the two Cucurbita plants is ‘PUV008,’ representative tissue of ‘PUV008’ having been deposited under NCIMB No. 43740. In some embodiments, the method further comprises: e) crossing the selected progeny plant having the determinate growth habit another Cucurbita plant, or with itself, and selecting resulting subsequent progeny for the determinate growth habit. In some embodiments, the method further comprises: e) propagating the selected progeny plant via seed, cutting, or tissue culture.

In some embodiment, a method for producing a Cucurbita plant having a determinate growth habit comprises: propagating a vegetative cutting from a stock Cucurbita plant, thereby producing the Cucurbita plant having a determinate growth habit, wherein the stock Cucurbita plant has the a determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740. In some embodiments, said plant breeding technique is recurrent selection, mass selection, hybridization, open-pollination, backcrossing, or pedigree breeding.

In some embodiments, a method for producing a seed that develops into a Cucurbita plant having a determinate growth habit comprises: crossing a first Cucurbita plant with a stock Cucurbita plant, thereby producing the seed that develops into the Cucurbita plant having the determinate growth habit; wherein the first Cucurbita and/or the stock Cucurbita plant has the determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740. In some embodiments, said plant breeding technique is recurrent selection, mass selection, hybridization, open-pollination, backcrossing, or pedigree breeding. In some embodiments, said plant breeding technique is mutation breeding, and wherein said mutation breeding selects for a mutation that is spontaneous or artificially induced. In some embodiments, said plant breeding technique is marker enhanced selection.

In some embodiments, a method of determining the genotype of the Cucurbita plant designated PUV008 comprises obtaining a sample of nucleic acids from said plant and detecting in said nucleic acids a plurality of polymorphisms.

In some embodiments, a method of introducing a mutation into the genome of a PUV008 plant comprises mutagenesis of the plant, or plant part of Cucurbita variety PUV008, wherein said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.

The present disclosure provides a method of genetically modifying the plant of Cucurbita variety PUV008, wherein said genetic modification is selected from agrobacterium-mediated gene transfer, protoplast transformation, or biolistic transformation. In some embodiments, a plant is produced by the method of genetically modifying the plant of Cucurbita variety PUV008, wherein said plant has the determinate growth habit.

### BRIEF DESCRIPTION OF THE DRAWINGS

FIG. 1 depicts a breeding strategy of introducing a determinate habit into an indeterminate Cucurbita moschata plant. A donor line (A) was crossed with an acceptor line (B) by carrying the determinate habit feature into a butternut Cucurbita moschata plant (B). Two recurrent backcrosses were followed with a second Cucurbita moschata line (C).

FIG. 2A-2B depicts the determinate trait of the C. moschata plant with determinacy obtained (FIG. 2B) in comparison to the indeterminate check variety (FIG. 2A).

FIG. 3 depicts length comparison of 30 first internodes between indeterminate Cucurbita plants and determinate Cucurbita plants with QTL associated with determinate growth habit.

FIG. 4 depicts average length of internodes average between indeterminate Cucurbita plants and determinate Cucurbita plants with QTL associated with determinate growth habit.

FIG. 5 depicts average number of internodes between indeterminate Cucurbita plants and determinate Cucurbita plants with QTL associated with determinate growth habit.

FIG. 6 shows a picture of the indeterminate plant (left) and the determinate plant (right). While the main stem and lateral shoots of the indeterminate plant has long internodes, the main stem and lateral shoots of the determinate plant produces short internodes (the lateral shoots of the determinate plants have been cut to better see the short internodes).

FIG. 7 shows an entire view of the indeterminate plant (left) and the determinate plant (right).

FIG. 8 shows an entire view of the determinate plant with all the leaves removed (left) and the indeterminate plant with all the leaves removed (right). While the main stem and lateral shoots of the indeterminate plant has long internodes, the main stem and lateral shoots of the determinate plant produces short internodes.

FIG. 9 depicts the phenotypic traits to be characterized of the Cucurbita plant with determinacy obtained.

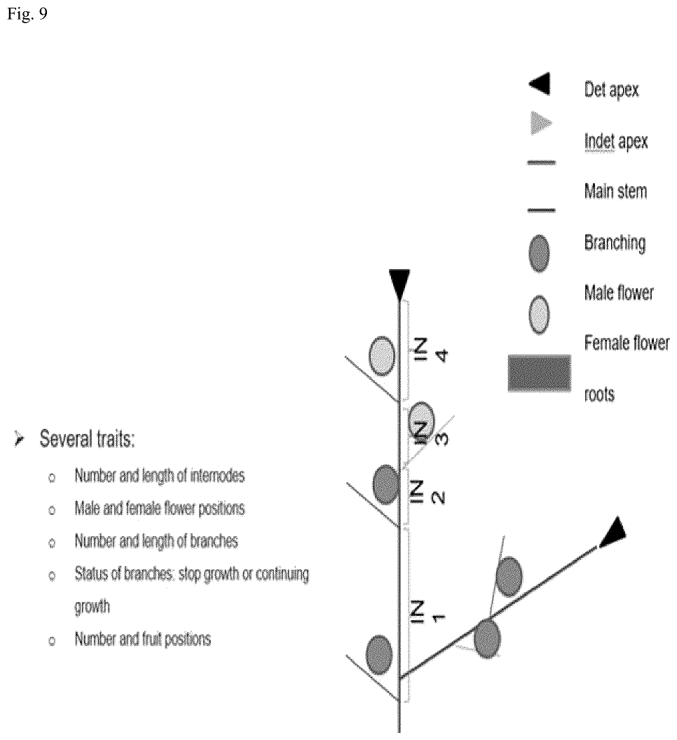

FIG. 10 depicts multiple sequence alignment results of the 5′ UTR of CmoCh15G012090. HMC sequence obtained from the plants of the present disclosure (PUV008) shows a genetic modification with a portion of the 5′ UTR of CmoCh15G012090 gene deleted. Sequences of the deleted 5′UTR of CmoCh15G012090 gene (i.e., Bu, CmoM-1, CmoM-2, CmoM-3, and Cmo-M4 described in Wang et al., 2022) are presented as a positive control to show similar 5′ UTR deletion.

FIG. 11 presents physical positions of SNP markers on chromosome 15 of the publicly-available C. moschata genome map (Rifu) along with candidate gene CmoCh15G012090 involved with growth habit determinacy.

FIG. 12 shows a picture of PUV008 fruit before at the pre-harvesting stage.

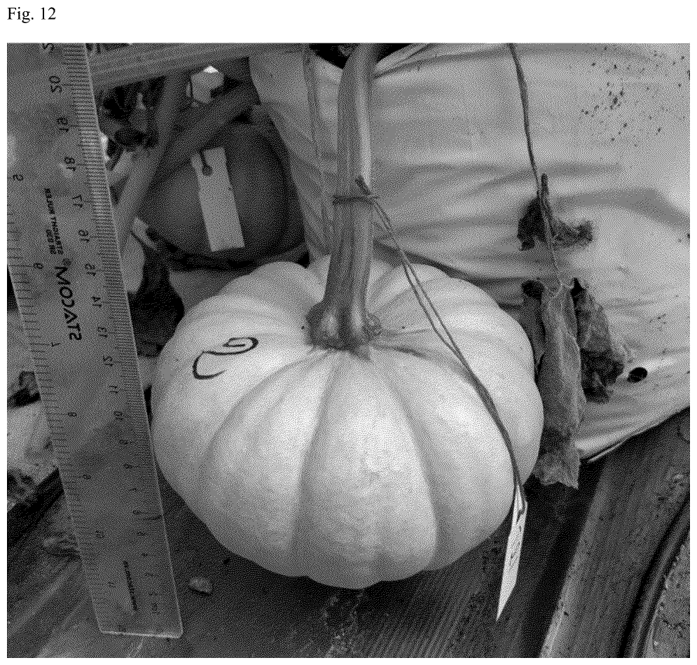

### BRIEF DESCRIPTION OF THE SEQUENCE LISTING

SEQ ID NO:1 corresponds to the sequence of marker SQ-0018902.

SEQ ID NO:2 corresponds to the sequence of marker SQ-0018903.

SEQ ID NO:3 corresponds to the sequence of marker SQ-0018904.

SEQ ID NO:4 corresponds to the sequence of marker SQ-0018907.

SEQ ID NO:5 corresponds to the sequence of marker SQ-0018909.

SEQ ID NO:6 corresponds to the sequence of marker SQ-0018839, which is identical to SQ-0018907 (SEQ ID NO: 4). SEQ ID Nos:4 and 6 can be interchangeably used herein.

SEQ ID NO:7 corresponds to the sequence of marker SQ-0018902-N.

SEQ ID NO:8 corresponds to the sequence of marker SQ-0018903-N.

SEQ ID NO:9 corresponds to the sequence of marker SQ-0018904-N.

SEQ ID NO:10 corresponds to the sequence of marker SQ-0018907-N.

SEQ ID NO:11 corresponds to the sequence of marker SQ-0018909-N.

SEQ ID NO:12 corresponds to the sequence of marker SQ-0018814-N.

SEQ ID NO:13 corresponds to 5′ UTR of CmoCh15G012090 (CmoCh15G012090).

SEQ ID NO:14 corresponds to the deletion of partial 5′UTR of CmoCh15G012090 gene (HMC sequence).

SEQ ID NO:15 corresponds to the deletion of partial 5′UTR of CmoCh15G012090 gene (Bu from Wang).

SEQ ID NO:16 corresponds to the deletion of partial 5′UTR of CmoCh15G012090 gene (CmoM-1).

SEQ ID NO:17 corresponds to the deletion of partial 5′UTR of CmoCh15G012090 gene (CmoM-2).

SEQ ID NO:18 corresponds to the deletion of partial 5′UTR of CmoCh15G012090 gene (CmoM-3).

SEQ ID NO:19 corresponds to the deletion of partial 5′UTR of CmoCh15G012090 gene (CmoM-4).

### DETAILED DESCRIPTION

All publications, patents and patent applications, including any drawings and appendices, herein are incorporated by reference to the same extent as if each individual publication or patent application was specifically and individually indicated to be incorporated by reference.

The following description includes information that may be useful in understanding the present disclosure. It is not an admission that any of the information provided herein is prior art or relevant to the presently claimed disclosures, or that any publication specifically or implicitly referenced is prior art.

### Definitions

As used herein, the verb “comprise” as is used in this description and in the claims and its conjugations are used in its non-limiting sense to mean that items following the word are included, but items not specifically mentioned are not excluded.

As used herein, the term “a genus Cucurbita plant” refers to any plant belonging to the genus Cucurbita.

As used herein, the term “plant part” refers to any part of a plant including but not limited to the shoot, root, rootstock, scion, stem, seeds, stipules, leaves, petals, flowers, ovules, bracts, branches, petioles, internodes, bark, pubescence, tillers, rhizomes, fronds, blades, pollen, stamen, fruits, and the like. The two main parts of plants grown in some sort of media, such as soil, are often referred to as the “above-ground” part, also often referred to as the “shoots”, and the “below-ground” part, also often referred to as the “roots”.

The term “a” or “an” refers to one or more of that entity; for example, “a plant” refers to one or more plants or at least one plant. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid,” “nucleotide,” and “polynucleotide” are used interchangeably.

As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

As used herein, the term “homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. In some embodiments, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%.

As used herein, the term “nucleotide change” refers to, e.g., nucleotide substitution, deletion, and/or insertion, as is well understood in the art. For example, mutations containing alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “at least a portion” of a nucleic acid or polypeptide means a portion having the minimal size characteristics of such sequences, or any larger fragment of the full length molecule, up to and including the full length molecule. For example, a portion of a nucleic acid may be at least 1 nucleotide, at least 2 nucleotide, at least 3 nucleotide, at least 4 nucleotide, at least 5 nucleotide, at least 6 nucleotide, at least 7 nucleotide, at least 8 nucleotide, at least 9 nucleotide, at least 10 nucleotide, at least 11 nucleotide, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 22 nucleotides, at least 24 nucleotides, at least 26 nucleotides, at least 28 nucleotides, at least 30 nucleotides, at least 32 nucleotides, at least 34 nucleotides, at least 36 nucleotides, at least 38 nucleotides, at least 40 nucleotides, at least 45 nucleotides, at least 50 nucleotides, at least 55 nucleotides, and so on, going up to the full length nucleic acid. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as hybridization probe may be as short as 12 nucleotides; in one embodiment, it is 20 nucleotides. A portion of a polypeptide useful as an epitope may be as short as 4 amino acids. A portion of a polypeptide that performs the function of the full-length polypeptide would generally be longer than 4 amino acids.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm, for example, NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx, and the Clustal W and Clustal X (Larkin et al. 2007 Bioinformatics, 23, 2947-294, Clustal W and Clustal X version 2.0) as well as Clustal Omega. Unless otherwise stated, references to sequence identity used herein refer to the Clustal Omega.

As used herein, the term “substantially complementary” means that two nucleic acid sequences have at least about 65%, about 70% or 75%, about 80% or 85%, about 90% or 95%, or about 98% or 99%, sequence complementarities to each other. This means that primers and probes must exhibit sufficient complementarity to their template and target nucleic acid, respectively, to hybridize under stringent conditions. Therefore, the primer and probe sequences need not reflect the exact complementary sequence of the binding region on the template and degenerate primers can be used. For example, a non-complementary nucleotide fragment may be attached to the 5′-end of the primer, with the remainder of the primer sequence being complementary to the strand. Alternatively, non-complementary bases or longer sequences can be interspersed into the primer, provided that the primer has sufficient complementarity with the sequence of one of the strands to be amplified to hybridize therewith, and to thereby form a duplex structure which can be extended by the polymerizing means. The non-complementary nucleotide sequences of the primers may include restriction enzyme sites. Appending a restriction enzyme site to the end(s) of the target sequence would be particularly helpful for cloning of the target sequence. A substantially complementary primer sequence is one that has sufficient sequence complementarity to the amplification template to result in primer binding and second-strand synthesis. The skilled person is familiar with the requirements of primers to have sufficient sequence complementarity to the amplification template.

As used herein, the terms “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer can be single stranded for maximum efficiency in amplification. In some embodiments, the primer is an oligodeoxyribonucleotide. The primer must be sufficiently long to prime the synthesis of extension products in the presence of the agent for polymerization. The exact lengths of the primers will depend on many factors, including temperature and composition (A/T en G/C content) of primer. A pair of primers consists of one forward and one reverse primer as commonly used in the art of DNA amplification such as in PCR amplification.

As used herein, the term “inbred”, “inbred plant” is used in the context of the present disclosure, this also includes any single gene conversions of that inbred. The term single allele converted plant as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “sample” includes a sample from a plant, a plant part, a plant cell, or from a transmission vector, or a soil, water or air sample.

As used herein, the term “biological sample” includes a DNA sample, a RNA sample, and/or a protein sample extracted from any part of a plant (e.g. leaf, fruit, stem)

As used herein, the term “progeny” or “progenies” refers to the descendants of one or more of the parental lines and includes an F1 plant produced from the cross of two parental plants where at least one parental plant includes a Cucurbita plant disclosed herein and progeny further includes, but is not limited to, subsequent F2, F3, F4, F5, F6, F7, F8, F9, and F10 generational crosses with the recurrent parental line.

As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “genotype” refers to the genetic makeup of an individual cell, cell culture, tissue, organism (e.g., a plant), or group of organisms.

As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus. As used herein, the term “heterozygous” refers to the presence of different alleles (forms of a given gene) at a particular gene locus.

As used herein, the terms “homolog” or “homologue” refer to a nucleic acid or peptide sequence which has a common origin and functions similarly to a nucleic acid or peptide sequence from another species.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by different sequences. In some embodiments, a specific place or places, or a site on a chromosome where a gene or molecular marker, such as a SNP, is found.

As used herein, the terms “genetically linked to” refers to the situation wherein the two genetic elements are segregating together over one or more generation. More specifically, by “a QTL associated with a determinate growth habit genetically linked to a marker”, it is meant that the QTL associated with a determinate growth habit is segregating with the marker over one or more generation. In some embodiments, distances between loci are usually measured by frequency of crossing-over between loci on the same chromosome. The farther apart two loci are, the more likely that a crossover will occur between them. Conversely, if two loci are close together, a crossover is less likely to occur between them. As a rule, one centimorgan (cM) is equal to 1% recombination between loci (markers). For example, the genetic distance between the QTL and the marker is about 4.9 cM, 4.8 cM, 4.7 cM, 4.6 cM, 4.5 cM, 4.4 cM, 4.3 cM, 4.2 cM, 4.1 cM, 4.0 cM, about 3.9 cM, 3.8 cM, 3.7 cM, 3.6 cM, 3.5 cM, 3.4 cM, 3.3 cM, 3.2 cM, 3.1 cM, 3.0 cM, about 2.9 cM, 2.8 cM, 2.7 cM, 2.6 cM, 2.5 cM, 2.4 cM, 2.3 cM, 2.2 cM, 2.1 cM, 2.0 cM, about 1.9 cM, about 1.8 cM, about 1.7 cM, about 1.6 cM, about 1.5 cM, about 1.4 cM, about 1.3 cM, about 1.2 cM, about 1.1 cM, about 1.0 cM, about 0.9 cM, about 0.8 cM, about 0.7 cM, about 0.6 cM, about 0.5 cM, about 0.4 cM, about 0.3 cM, about 0.2 cM, about 0.1 cM, or less than 0.1 cM.

As used herein, the term “mass selection” refers to a form of selection in which individual plants are selected and the next generation propagated from the aggregate of their seeds.

As used herein, the terms “mutant” or “mutation” refer to a gene, cell, or organism with an abnormal genetic constitution that may result in a variant phenotype.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein when discussing plants, the term “ovule” refers to the female gametophyte, whereas the term “pollen” means the male gametophyte.

As used herein, the term “phenotype” refers to the observable characters of an individual cell, cell culture, organism (e.g., a plant), or group of organisms which results from the interaction between that individual's genetic makeup (i.e., genotype) and the environment.

As used herein, the term “plant line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selling). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

As used herein, the term “self-crossing”, “self-pollinated” or “self-pollination” means the pollen of one flower on one plant is applied (artificially or naturally) to the ovule (stigma) of the same or a different flower on the same plant.

As used herein, the term “variety” refers to a subdivision of a species, consisting of a group of individuals within the species that are distinct in form or function from other similar arrays of individuals.

As used herein, “quantitative trait locus (QTL)” refer to a genetic domain that effects a phenotype that can be described in quantitative terms and can be assigned a “phenotypic value” which corresponds to a quantitative value for the phenotypic trait. It means a locus that controls to some degree numerically representable traits that are usually continuously distributed. A QTL may for instance comprise one or more genes of which the products confer a desired trait(s). Alternatively, a QTL may for instance comprise regulatory genes or sequences of which the products influence the expression of genes on other loci in the genome of the plant thereby conferring the desired trait(s). The QTLs of the present disclosure may be defined by indicating their genetic location in the genome that is associated with the desired trait(s) using one or more molecular genomic markers. One or more markers, in turn, indicate a specific locus. When a QTL can be indicated by multiple markers the genetic distance between the end-point markers is indicative of the size of the QTL.

The term “marker” or “molecular marker” refers to a genetic locus (a “marker locus”) used as a point of reference when identifying genetically linked loci such as a QTL. In some embodiments, the term can refer to a nucleotide sequence or a fragment of such sequence, e.g., a single nucleotide polymorphism (SNP), used as a point of reference at an identifiable physical location on a chromosome (e.g. restriction enzyme cutting site, gene) whose inheritance can be tracked. In some embodiments, markers can be derived from genomic nucleotide sequences or from expressed nucleotide sequences (e.g., from a spliced RNA, cDNA, etc.). In some embodiments, the term can also refer to nucleic acid sequences used as a molecular marker probe, primer, primer pair, or a molecule that can be used to identify the presence of a marker locus, e.g., a nucleic acid probe that is complementary to a marker locus sequence, and is capable of amplifying sequence fragments using PCR and modified PCR reaction methods.

For example, a molecular marker assay, such as PCR, KASP, or SSR can be used to identify whether a certain DNA sequence or SNP, for example, is present in a sample of DNA. For example, a marker assay can include a molecular marker assay, e.g., KASP assay, which can be used to test whether a cultivated, landrace, heirloom, or pureline plant has a SNP associated with an expression of a trait from DNA extracted from the plant. Markers corresponding to genetic polymorphisms between members of a population can be detected by methods commonly used in the art including, PCR-based sequence specific amplification methods, detection of restriction fragment length polymorphisms (RFLPs), detection of amplified variable sequences of the plant genome, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD). Other examples of such methods are using sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the person skilled in the art using common molecular techniques. It is noted in this respect that specific positions in a chromosome can indeed be defined with respect to markers, such as SNPs, insofar as the flanking sequences of said markers are defined in order to unambiguously position them on the genome. In some embodiments, the present inventors have used SNPs markers, identified by their flanking sequences, present in the Cucurbita (e.g. C. moschata) genome, to discriminate between introgressed and endogenously residing sequences and to track down the introgressed sequences conferring the determinate growth habit.

The term “marker assisted selection” or “MAS” is a process of identifying and using the presence (or absence) of one or more molecular markers, e.g., a SNP, associated with a particular locus or to a particular chromosome region, to select plants for the presence of the specific locus. For example, the presence of a SNP known to be associated with a volatile compound can be used to detect and/or select common bean plants expressing the volatile compound of interest. MAS can be used to quickly introgress simply inherited traits, test early generations, breakup linkage drag, pyramid genes, and/or authenticate the identity of a cultivar.

The term “marker assisted selection breeding” refers to the process of selecting a desired trait or traits in a plant or plants by detecting one or more nucleic acids from the plant, where the nucleic acid is linked to the desired trait, and then selecting the plant or germplasm possessing those one or more nucleic acids.

The term “single-nucleotide polymorphism” or “SNP” is a variation in a single nucleotide (A, T, C, or G) that occurs at a specific position in a DNA sequence of a genome, where each variation is present to some appreciable degree within members of the same species or a paired chromosome (e.g., >1%). A SNP serves as a molecular marker used to assist in locating genes associated with certain traits expressed by genes related to the SNP. For example, at a specific base position in a genome, the base C may appear in a majority of the members of the same species, but in a minority of members of that same species, the position is occupied by the base A. The SNP at this specific base position, and the two possible nucleotide variations—C or A—are alleles for this base position. A SNP may fall within coding sequences of a gene, a non-coding region of a gene, or in intergenic regions.

The term “introgress” or “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene or a selected allele of a marker or QTL.

As used herein, “allele” refers to an alternative nucleic acid sequence at a particular locus. The length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population.

As used herein, “selecting” or “selection” in the context of marker-assisted selection or breeding refer to the act of picking or choosing desired individuals, normally from a population, based on certain pre-determined criteria.

As used herein, “mapping” is the process of defining the linkage relationships of loci through the use of genetic markers, populations segregating for the markers, and standard genetic principles of recombination frequency.

As used herein, a “genetic map” is the relationship of genetic linkage among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group or chromosome where a specified marker can be found within a given species. In contrast, a “physical map” of the genome refers to absolute distances (for example, measured in base pairs or isolated and overlapping contiguous genetic fragments, e.g., contigs). In general, the closer two markers or genomic loci are on the genetic map, the closer they lie to one another on the physical map. A physical map of the genome does not take into account the genetic behavior (e.g., recombination frequencies) between different points on the physical map. A lack of precise proportionality between genetic distances and physical distances can exist due to the fact that the likelihood of genetic recombination is not uniform throughout the genome; some chromosome regions are cross-over “hot spots,” while other regions demonstrate only rare recombination events, if any. Genetic mapping variability can also be observed between different populations of the same crop species. In spite of this variability in the genetic map that may occur between populations, genetic map and marker information derived from one population generally remains useful across multiple populations in identification of plants with desired traits, counter-selection of plants with undesirable traits and in MAS breeding. As one of skill in the art will recognize, recombination frequencies (and as a result, genetic map positions) in any particular population are not static. The genetic distances separating two markers (or a marker and a QTL) can vary depending on how the map positions are determined. For example, variables such as the parental mapping populations used, the software used in the marker mapping or QTL mapping, and the parameters input by the user of the mapping software can contribute to the QTL marker genetic map relationships. However, it is not intended that the disclosure be limited to any particular mapping populations, use of any particular software, or any particular set of software parameters to determine linkage of a particular marker or chromosome interval with a desired phenotype. It is well within the ability of one of ordinary skill in the art to extrapolate the novel features described herein to any gene pool or population of interest, and using any particular software and software parameters.

As used herein, a “chromosomal region” or “chromosomal interval” delimited by two markers (e.g. SNPs) X and Y refers to the section of the chromosome or linkage group lying between the positions of these two markers and comprising said markers, therefore the nucleotide sequence of this chromosomal region or interval begins with the nucleotide corresponding to marker X and ends with the nucleotide corresponding to marker Y, i.e. the markers are comprised within the region or interval they delimit, in the sense of the disclosure.

As used herein, a “desirable trait” or “desirable traits” that may be introduced into plants by breeding may be directed to the fruit or the plant. Desirable traits to be introduced into plants and fruit may be independently selected. Desirable fruit traits, e.g. as displayed by agronomically elite lines or cultivars, and that may be independently selected include, but are not limited to: fruit size, shape, color, surface appearance; seed number, seed size, locule number; pericarp thickness and toughness; taste, bitterness, the presence of tubercles, and shelf life. Desirable plant traits, e.g. as displayed by agronomically elite lines or cultivars, and that may be independently selected include, but are not limited to: plant vigor, leaf shape, leaf length, leaf color, plant height, plant growth habit (determinate, semi-determinate or indeterminate), fruit set timing, time to maturity, adaptation to field growth, adaptation to greenhouse growth, and resistance to one or more diseases or disease causing organisms such as Bacterial wilt (Erwinia tracheiphila), Alternaria leaf blight (Alternaria cucumerina), Downy mildew (Pseudoperonospora cubensis), Powdery mildew (Erysiphe spp. or Sphaerotheca spp.) Squash Mosaic Virus, Zucchini Yellow Mosaic Virus, Phytophthora blight (Phytophthora capsici), Tomato Leaf Curl New Delhi Virus (ToLCNDV), and the like. In some embodiments, any combination of desirable fruit traits, plant traits, or plant and fruit traits may be combined with a determinate growth habit trait. The resulting agronomically elite Cucurbita plants of the present disclosure surprisingly display such agronomic traits in combination with a determinate growth habit, while lacking deleterious traits.

As used herein, “polymorphism” means the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found, or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs) a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, dsRNA, a transcriptional profile, and a methylation pattern may comprise polymorphisms. In addition, the presence, absence, or variation in copy number of the preceding may comprise a polymorphism.

### Cucurbita

Cucurbita is a plant genus of the Cucurbitaceae family. Most Cucurbita species are herbaceous annual vines that grow several meters in length and have tendrils, but non-vining “bush” cultivars of C. pepo and C. maxima have also been developed. Many species have large, yellow or white flowers. The stems are hairy and pentangular. Tendrils are present at 900 to the leaf petioles at nodes. Leaves are exstipulate alternate simple palmately lobed or palmately compound. The flowers are unisexual, with male and female flowers on different plants (dioecious) or on the same plant (monoecious). The female flowers have inferior ovaries. The fruit is often a kind of berry called a pepo.

Cucurbita genus plants include at least the following 13 species groups: C. argyrosperma or C. mixta group (e.g. C. kellyana, C. palmeri, C. sororia species), C. digitata group (e.g. C. californica, C. cordata, C. cylindrata and C. palmata species), C. ecuadorensis group, C. ficifolia group, C. foetidissima group (e.g. C. scabridifolia species), C. galeottii group, C. lundelliana group, C. maxima group (e.g. C. andreana species), C. moschata group, C. okeechobeensis group (e.g. C. martinezii species), C. pedatifolia group (e.g. C. moorei species), C. pepo group (e.g. C. fraterna and C. texana species), and C. radicans group (e.g. including C. gracillor species).

The five domesticated species are Cucurbita argyrosperma, C. ficifolia, C. maxima, C. moschata, and C. pepo. All of these can be treated as winter squash because the full-grown fruits can be stored for months; however, C. pepo includes some cultivars that are better used only as summer squash.

### Squash

Squash is the common name for a collection of plants that produce edible seeds, fruits and flowers. Squashes generally refer to four species of the genus Cucurbita native to Mexico and Central America, also called marrows depending on variety or the nationality of the speaker. It is also natively grown in other parts of North America, and in Europe, India, and Australia. In North America, squash is loosely grouped into summer squash or winter squash, as well as autumn squash (another name is cheese squash) depending on whether they are harvested as immature vegetables (summer squash) or mature vegetables (autumn squash or winter squash). Gourds are from the same family as squashes. Well known types of squash include the pumpkin and zucchini. Giant squash are derived from Cucurbita maxima and are routinely grown to weights nearing those of giant pumpkins. Non-limiting examples of squash species include, C. maxima (winter squash), C. mixta (cushaw squash), C. moschata (winter crookneck squash, e.g., butternut squash), C. pepo var. pepo (most pumpkins, e.g. Halloween type pumpkins, acorn squash, summer squash (bush summer squash zucchini), ambercup squash, autumn cup squash, banana squash, buttercup squash, carnival squash, delicata squash, gold nugget squash, kabocha squash, spaghetti squash, sweet dumpling squash, hubbard squash, and turban squash).

Winter squashes are the mature fruits of three Cucurbit species: Cucurbita maxima, Cucurbita moschata and Cucurbita pepo. Fruit from winter squash varieties are grown to physiological maturity and typically stored for consumption during the winter months or used for ornamental purposes. Examples of common winter squashes are acorn, butternut, hubbard, and spaghetti squash, as well as the Halloween type pumpkins. Cucurbita maxima is one of the most diverse domesticated species, perhaps with more cultivated forms than any other crop. This species originated in South America from the wild C. maxima ssp. andreana over 4000 years ago. Different squash types of this species were introduced into North America as early as the 16th century. By the early 19th century, at least three varieties are known to have been commercially introduced in North America from seeds obtained from Native Americans. Secondary centers of diversity include India, Bangladesh, Bunna, and possibly the southern Appalachians. Non-limiting examples of Cucurbita maxima include, Banana squash, Buttercup squash, Jarrandale pumpkin, Kabocha (e.g. KURIJIMAN), Lakota squash, Arikara squash, and Hubbard squash. Candyroaster landrace Cucurbita moschata is a species that includes some varieties of squash and pumpkin. C. moschata squash are generally more tolerant of hot, humid weather than C. maxima or C. pepo. They also generally display a greater resistance to disease and insects, especially to the squash vine borer. Non-limiting examples of C. moschata include, butternut squash (e.g. C. moschata butternut type, C. moschata long neck butternut type), C. moschata flat round type, C. moschata round to oblate type, Dickinson field pumpkin, Kentucky field pumpkin, Long Island cheese pumpkin, Calabaza pumpkin, Seminole pumpkin, Neck pumpkin, and Long of Naples squash. Cucurbita pepo is the main economic squash species. It includes varieties of squash, gourd, and pumpkin. Non-limiting example of Cucurbita pepo include, Acorn squash, Delicata squash, Gem squash, Heart of gold squash, Pattypan squash, Some types of Pumpkin, Spaghetti squash, Sweet dumpling squash, Yellow crookneck squash, Yellow summer squash, and Zucchini.

Most summer squash varieties are Cucurbita pepo, and their fruits are typically harvested and consumed at an immature stage. The flowers of summer squash can also be harvested for consumption. There are many types of summer squash, including yellow crookneck, yellow straightneck, scallop, Lebanese, and green and gray zucchini. Green zucchini is the type of C. pepo squash preferred by consumers in Europe and many parts of the North America, as well as in other regions. Unlike winter squashes, summer squash fruit have a short shelf life, and are typically consumed within days of harvest. Because of the extended ability to ship produce over long distances there are some markets where the terms “summer” and “winter” squash no longer reflect a restriction on availability and all types can be found in these markets year round.

### Semi-Determinate or Determinate Growth Habit

The present disclosure teaches that a semi-determinate or determinate growth habit is a desired trait that is introgressed into a Cucurbita plant.

While indeterminate growth is growth that is not terminated, determinate growth stops once a genetically pre-determined structure has completely formed. Plant that grows and produces flowers and fruit until killed by frost or some other external factor is called indeterminate.

For example, the term is applied to indeterminate Cucurbita varieties which continuously produce internodes (i.e. leading to long internodes) and flowers (male and female) associated to those internodes, producing fruit throughout the growing season, and in contrast to a determinate Cucurbita plant, which show a determinate number of internodes (e.g. short internodes) and flowers (male and female) associated to those internodes.

Determinate Cucurbita plants stop growing (i.e. terminate) and show an apex that stopped (i.e. determinate growth) after a given number of internodes, and plants themselves are generally upright and bushy. Determinate Cucurbita plants thus allow a concentrated fruit harvest.

Indeterminate Cucurbita plants form flowers and fruits at the point where a leaf grows out from the stem. The shoots keep growing while the flowers and fruits are formed. Plants grow flat on the ground and form vines. Indeterminate Cucurbita plants thus allow a fruit harvest throughout the season, i.e. a non-concentrated fruit harvest.

Semi-determinate Cucurbita plants grow very similar to the fully determinate Cucurbita plants, which terminates (i.e. stop growing) and show an apex that stopped (i.e. determinate growth) after a given number of internodes. The main difference between the determinate and semi-determinate Cucurbita plants is that the semi-determinate plants grow further before terminating. That is, the semi-determinate Cucurbita plants terminate like the determinate Cucurbita plants, but show longer internodes than the determinate Cucurbita plants.

Most of the plants in the Cucurbita family form short twisted branches called tendrils. Tendrils wrap around stems or stakes as the plant grows. In general, pumpkins, squash, and gourds of Cucurbita plants have shallow roots that grow in a wide area. Roots can grow along a vine as it lies on the ground. Roots form on the stem at the place where a leaf grows.

In some embodiments, Cucurbita plants of the present disclosure have a semi-determinate or determinate growth habit. In some embodiments, the determinate growth habit is obtained by possessing a QTL associated with the determinate growth trait at a homozygous manner. In some embodiments, the semi-determinate growth habit is obtained by possessing a QTL associated with the determinate growth trait at a heterozygous manner. The trait is introgressed by a traditional breeding technique (i.e. crossing).

The term “determinate” or “determinacy” refers to Cucurbita plants with short internodes and a limited number of internodes. Herein, the botanical definition of determinate is used to refer to a growth habit in which vegetative growth stops with an inflorescence or other reproductive structure formed at a terminal bud. Conventional Cucurbita plants used for processing are indeterminate. Indeterminate Cucurbita plants generally produce long internodes and continue producing flowers and fruits, while determinate Cucurbita plants produce relatively short internodes and do not continuously produce flowers and fruit after a certain point in development. Semi-determinate Cucurbita plants produce moderately long internodes, but shorter than those of indeterminate Cucurbita plants. According to one aspect of the present disclosure, the internode number of the determinate plant can be about 15 to 35, while the indeterminate plant has about 45 to 65 internodes, or more. In other embodiments, the determinate Cucurbita plants may have less than 15 internodes, and the indeterminate Cucurbita plants may have more than 65 internodes In general, Cucurbita plants set fruits at flowering nodes.

In one embodiment, the semi-determinate or determinate Cucurbita plants has substantially shorter internodes than the indeterminate Cucurbita plants. In another aspect of this embodiment, a semi-determinate or determinate Cucurbita plants has substantially a limited number of internodes when compared to the indeterminate Cucurbita plants. In further embodiment, determinate Cucurbita plants have shorter internodes than semi-determinate Cucurbita plants.

In some embodiments, Cucurbita plants carrying the genetic determinacy have a limited number of internodes and shorter internodes compared to indeterminate Cucurbita plants. Thus, Cucurbita plants of the present disclosure are the compact plants that allow for concentrated harvest and increased plant density in the field and greenhouse, thereby benefitting breeders and growers.

In some embodiments, Cucurbita plants of the present disclosure has the constrained fruit set timing that leads to increased uniformity of fruit maturity at harvest. In some embodiments, Cucurbita plants of the present disclosure with the determinate growth habit tend to produce all their fruits at about the same time. In further embodiments, Cucurbita plants of the present disclosure with determinate growth habit solve the issue of immature fruit being harvested with mature fruit due to discorded fruit set timing among fruits.

Teachings concerning ‘short internode’ trait and/or ‘terminal flower formation’ trait in Cucurbita especially Melon, Watermelon and Cucumber are known in the art. Thus, references (Wen et al, 2019a; Gebremeskel et al, 2020; Zhao et al, 2018; Zhang et al, 2020; Njogu et al, 2020; Zhao et al, 2018; Dong et al, 2018, Zhang et al, 2019, Wei et al, 2019b, Yang et al, 2019, Hou et al, 2017; Xu et al, 2018) are incorporated herein by reference in its entirety.

Methods for Producing a Cucurbita Plant with a Desired Trait

The present disclosure provides a method for producing a Cucurbita plant with a desired trait (i.e. a semi-determinate or determinate growth habit) and using the plants to identify genotypes associated with phenotypes of interest (i.e. semi-determinate or determinate growth habit) wherein the Cucurbita plant is assayed with at least one marker and associating the at least one marker with at least one phenotypic trait. The genotype of interest can then be used to make decisions in a plant breeding program. Such decisions include, but are not limited to, selecting among new breeding populations which population has the highest frequency of favorable nucleic acid sequences based on historical genotype and agronomic trait associations, selecting favorable nucleic acid sequences among progeny in breeding populations, selecting among parental lines based on prediction of progeny performance, and advancing lines in germplasm improvement activities based on presence of favorable nucleic acid sequences. Non-limiting examples of germplasm improvement activities include line development, hybrid development, transgenic event selection, making breeding crosses, testing and advancing a plant through self-fertilization, using plants for transformation, using plants for candidates for expression constructs, and using plants for mutagenesis.

Non-limiting examples of breeding decisions include progeny selection, parent selection, and recurrent selection for at least one haplotype. In another aspect, breeding decisions relating to development of plants for commercial release comprise advancing plants for testing, advancing plants for purity, purification of sublines during development, inbred development, variety development, and hybrid development. In yet other aspects, breeding decisions and germplasm improvement activities comprise transgenic event selection, making breeding crosses, testing and advancing a plant through self-fertilization, using plants for transformation, using plants for candidates for expression constructs, and using plants for mutagenesis.

In some embodiments, a method of mutagenesis described herein is selected from the group consisting of temperature, long term seed storage, somaclonal variation, radiation, chemical agents, targeting induced local lesions in genomes, site-directed mutagenesis, and genome editing. In some embodiments, the resulting plant produced by the method of mutagenesis comprises at least one genome mutation.

Plants of the present disclosure can be a Cucurbita plant that is determinate, semi-determinate, or indeterminate.

The present disclosure teaches that the segregation of a Cucurbita plant with determinate architecture in a Cucurbita plant introgressed with the determinate growth habit trait suggests a “codominant” or “incompletely dominant” determinism type of the trait, considering the overall plant architecture. In some embodiments, the determinate behavior of the apex (i.e. determinate growth) has a dominant determinism, whereas the internode length has an incomplete dominant or codominant determinism. A reasonable assumption is that the genetic mechanism is relatively simple, with one major QTL and unknown QTLs of minor effect(s).

The present disclosure teaches the genetic basis of the trait and the developed molecular markers for efficient introgression of the trait into elite germplasm through Marker Assisted Selection (MAS). In some embodiments, QTL associated with a determinate growth habit of the present disclosure may be introduced into an elite Cucurbita line. An “elite line” is any line that has resulted from breeding and selection for superior agronomic performance.

In another aspect, the Cucurbita plant can show a comparative determinate growth habit compared to an indeterminate control Cucurbita plant. In this aspect, a control Cucurbita plant will be genetically similar except for the allele or alleles of a QTL associated with a determinate growth habit of the present disclosure. Such plants can be grown under similar conditions with equivalent or near equivalent.

In one aspect, the semi-determinate or determinate Cucurbita plants have at least 500%, 400%, 300%, 200%, 150%, 100%, 90%, 80%, 70%, 60%, 50%, 45%, 40%, 30%, 25%, 20%, 15%, 10%, 5%, 2%, 1%, 0.5% or 0.1% of shorter internodes than the indeterminate Cucurbita plants.

In another aspect, the semi-determinate or determinate Cucurbita plants have less internodes than the indeterminate Cucurbita plants.

In another aspect, the semi-determinate or determinate Cucurbita plants have more compact than the indeterminate Cucurbita plants.

In another aspect, the semi-determinate or determinate Cucurbita plants have more increased plant density than the indeterminate Cucurbita plants.

In another aspect, the semi-determinate or determinate Cucurbita plants have more constrained fruit set timing than the indeterminate Cucurbita plants.

In another aspect, the semi-determinate or determinate Cucurbita plants have more synchronized fruit set timing than the indeterminate Cucurbita plants.

In another aspect, the semi-determinate or determinate Cucurbita plants have less discorded fruit set timing than the indeterminate Cucurbita plants.

In another aspect, the semi-determinate or determinate Cucurbita plants have increased uniformity of fruit maturity at harvest when compared to the indeterminate Cucurbita plants.

In another aspect, the semi-determinate or determinate Cucurbita plants have a concentrated fruit harvest when compared to the indeterminate Cucurbita plants.

In some embodiments, a determinate growth habit QTL allele or alleles can be introduced from any plant that contains that allele (donor) to any recipient Cucurbita plant. In one aspect, the recipient Cucurbita plant can contain additional loci associated with the determinate growth habit. In another aspect, the recipient Cucurbita plant can contain a transgene.

An allele of a QTL can, of course, comprise multiple genes or other genetic factors even within a contiguous genomic region, a chromosome, or linkage group, such as a haplotype. As used herein, an allele of a determinate growth habit locus can therefore encompass more than one gene or other genetic factor where each individual gene or genetic component is also capable of exhibiting allelic variation and where each gene or genetic factor is also capable of eliciting a phenotypic effect on the quantitative trait in question. In an aspect of the present disclosure, the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present in the disclosure can denote a haplotype within a haplotype window wherein a phenotype can be determinate growth habit. A haplotype window is a contiguous genomic region that can be defined, and tracked, with a set of one or more polymorphic markers wherein the polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus. Plants of the present disclosure may be homozygous or heterozygous at any particular determinate growth habit locus or for a particular polymorphic marker.

The present disclosure also provides a semi-determinate or determinate Cucurbita plant selected for by screening for determinacy or indeterminacy in the Cucurbita plant, the selection comprising interrogating genomic nucleic acids for the presence of a marker molecule that is genetically linked to an allele of a QTL associated with determinate growth habit in the Cucurbita plant, where the allele of a QTL is also located on a chromosome associated with determinate growth habit.

The present disclosure teaches that the determinacy in the determinate plant taught herein is due to a QTL located on chromosome 15 based on a publicly available Cucurbita moschata (Rifu) Genome database (cucurbitgenomics.org/organism/9), which is genetically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5). In some embodiments, said QTL conferring the determinate growth habit is located on chromosome 15 in a locus encompassing the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5). In some embodiments, said QTL conferring the determinacy is located on chromosome 15 within a chromosomal region delimited by markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5).

### Marker-Trait Associations

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs.

The genetic linkage of additional marker molecules can be established by a gene mapping model such as, without limitation, the flanking marker model reported by Lander et al., (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, XXell University, Ithaca, N.Y.).

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander et al. (1989), and further described by Arús and Moreno-González, Plant Breeding, Hayward, Bosemark, Romagosa (eds.) Chapman & Hall, London, pp. 314-331 (1993).

Many modifications and alternative approaches to interval mapping have been reported, including the use of non-parametric methods (Kruglyak et al., 1995 Genetics, 139:1421-1428). Multiple regression methods or models can also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen et al. (Jansen et al., 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng 1994). These models can be extended to multi-environment experiments to analyze genotype-environment interactions (Jansen et al., 1995 Theor. Appl. Genet. 91:33-3).

Selection of appropriate mapping populations is important to map construction. The choice of an appropriate mapping population depends on the type of marker systems employed (Tanksley et al., Molecular mapping in plant chromosomes. chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F₂ population is the first generation of selfing. Usually a single F₁ plant is selfed to generate a population segregating for all the genes in Mendelian (1:2:1) fashion. Maximum genetic information is obtained from a completely classified F₂ population using a codominant marker system (Mather, Measurement of Linkage in Heredity: Methuen and Co., (1938)). In the case of dominant markers, progeny tests (e.g. F₃, BCF₂) are required to identify the heterozygotes, thus making it equivalent to a completely classified F₂ population. However, this procedure is often prohibitive because of the cost and time involved in progeny testing. Progeny testing of F₂ individuals is often used in map construction where phenotypes do not consistently reflect genotype (e.g. determinate in growth habit) or where trait expression is controlled by a QTL. Segregation data from progeny test populations (e.g. F₃ or BCF₂) can be used in map construction. Marker-assisted selection can then be applied to cross progeny based on marker-trait map associations (F₂, F₃), where linkage groups have not been completely disassociated by recombination events (i.e., maximum disequilibrium).

Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so. Under conditions of tight linkage (i.e., about <10% recombination), dominant and co-dominant markers evaluated in RIL populations provide more information per individual than either marker type in backcross populations (Reiter et al., 1992 Proc. Natl. Acad. Sci. (USA) 89:1477-1481). However, as the distance between markers becomes larger (i.e., loci become more independent), the information in RIL populations decreases dramatically.

Backcross populations (e.g., generated from a cross between a successful variety (recurrent parent) and another variety (donor parent) carrying a trait not present in the former) can be utilized as a mapping population. A series of backcrosses to the recurrent parent can be made to recover most of its desirable traits. Thus a population is created consisting of individuals nearly like the recurrent parent but each individual carries varying amounts or mosaic of genomic regions from the donor parent. Backcross populations can be useful for mapping dominant markers if all loci in the recurrent parent are homozygous and the donor and recurrent parent have contrasting polymorphic marker alleles (Reiter et al., 1992). Information obtained from backcross populations using either codominant or dominant markers is less than that obtained from F₂ populations because one, rather than two, recombinant gametes are sampled per plant. Backcross populations, however, are more informative (at low marker saturation) when compared to RILs as the distance between linked loci increases in RIL populations (i.e. about 0.15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the polymorphic loci are expected to map to a selected region.

The development of massive number of molecular markers is the basis of molecular assisted selection (MAS), which is highly useful for plant breeding through the association between phenotypes and molecular markers. A huge number of molecular markers have been identified for constructing genetic map of plants, such as restriction fragment length polymorphism (RFLP), amplified fragment length polymorphism (AFLP), sequence-tagged sites (STS), cleaved amplified polymorphic sequences (CAP), inter simple sequence repeat (ISSR), and expressed sequence tag-simple sequence repeat (EST-SSR).

The present disclosure teaches that QTL mapping is an efficient way to identify candidate genes related with phenotypes in Cucurbita plants. However, fine mapping for traits of interest is difficult due to the low densities of the genetic markers in Cucurbita plants.

With the development of genotyping-by-sequencing (GBS), the resolution of QTL mapping has been improved significantly by the increasing numbers of markers. The present disclosure teaches Seq-BSA analysis, which is one way to discover genetic determinants underling phenotypic variants. Seq-BSA was used to identify SNP markers linked to determinate growth habit traits in the Cucurbita plants of the present disclosure. In some embodiments, combining QTL mapping with high-density genetic map and BSA are used to understand the genetic determinacy architecture of the Cucurbita plants with the determinate growth habit.

One skilled in the art will know how to use the seq-BSA method and associated strategies for QTL mapping (Mansfeld et al, 2018; Pujol et al, 2019, Ramos et al, 2020, Zhang et al, 2019, Zou et al, 2016; Song et al, 2017; Yoshitsu et al, 2017, Sun et al, 2018; Pang et al, 2018; Deokar et al, 2019; Zhang et al, 2018, Wang et al. 2016, each of which is incorporated herein by reference in its entirety).

The disclosure is directed to a method for identifying, detecting and/or selecting Cucurbita plants having the QTL associated with the determinate growth habit, either homozygously or heterozygously, on the basis of the allele detection of at least one of the marker of the present disclosure. When present homozygously, the QTL confers the determinate growth habit. When present heterozygously, the QTL confers the semi-determinate growth habit. In some embodiments, the markers are chosen amongst markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5) or any combinations thereof. In some embodiments, the detection and/or selection is made on the basis of specific allele of the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and/or SQ-0018909 (SEQ ID NO:5).

In some embodiments, plants bearing the introgressed sequences associated with determinate trait (i.e., determinate alleles from Table 1) are selected if at least one or more of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6), in a genetic material sample of the plant to be selected. In some embodiments, plants bearing the introgressed sequences associated with indeterminate determinate trait (i.e., indeterminate alleles from Table 1) are selected if all of the following alleles is detected: allele T of marker SQ-0018902-N (SEQ ID NO:7), allele G of marker SQ-0018903-N (SEQ ID NO:8), allele G of marker SQ-0018904-N (SEQ ID NO:9), allele G of marker SQ-0018907-N (SEQ ID NO:10), allele C of marker SQ-0018909-N (SEQ ID NO:11), allele C of marker SQ-0018914-N (SEQ ID NO:12), and allele C of marker SQ-0018839 (SEQ ID NO:6) in a genetic material sample of the plant to be selected. According to one embodiment, the allele(s) of interest which is/are detected is/are present homozygously in the selected plant, i.e. no other allele of said marker(s) is present. In such a case, it can be concluded that the plant bears the introgressed sequences that gives a trait of the determinate growth habit. Non-limiting detection methods are detailed above and applicable to this aspect of the disclosure.

In another embodiment, the allele(s) of interest which is/are detected is/are present heterozygously in the selected plant. In such a case, it can be concluded that the plant bears the introgressed sequences that gives a trait of the semi-determinate growth habit. Non-limiting detection methods are detailed above and applicable to this aspect of the disclosure.

In some embodiments, said Cucurbita plants are C. moschata, C. pepo or C. maxima plants.

The QTL responsible for the determinate growth habit can advantageously be introduced into Cucurbita plants or varieties, such as indeterminate C. moschata, C. pepo or C. maxima plants or varieties that contain other desirable genetic traits, such as resistance to another disease, early fruit maturation, drought tolerance, fruit shape, plant habit, internode length, androecy, gynoecy, and the like.

The markers of the disclosure can thus be used as detailed above, for selection plants or seed having the desired phenotype (i.e. determinate growth habit) or bearing introgression sequence conferring said phenotype when present homozygously. The same markers can be used for selection plants or seed having the desired phenotype (i.e. semi-determinate growth habit) or bearing introgression sequence conferring said phenotype when present heterozygously. According to one embodiment, the selection can be made on the basis of the presence of at least one or more of the following alleles of QTL markers for the determinate growth habit: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). According to another embodiment, the selection can be made on the basis of the presence of all of the following alleles of QTL markers for the indeterminate growth habit: allele T of marker SQ-0018902-N (SEQ ID NO:7), allele G of marker SQ-0018903-N (SEQ ID NO:8), allele G of marker SQ-0018904-N (SEQ ID NO:9), allele G of marker SQ-0018907-N (SEQ ID NO:10), allele C of marker SQ-0018909-N (SEQ ID NO:11), allele C of marker SQ-0018914-N (SEQ ID NO:12), and allele C of marker SQ-0018839 (SEQ ID NO:6). The presence of these alleles indeed confirms the presence of introgressed sequences at the chromosomal locus defined by said markers.

### Genetic Element Conferring Determinate Growth Habit

The present disclosure provide a candidate gene that may confer a determinate growth habit to Cucurbita plants of interest. This candidate gene is present in the QTL associated with the determinate growth habit trait, which is delimited by two SNP markers; (i) SQ-0018902-N (SEQ ID NO:7) and SQ-0018839 (SEQ ID NO:6); (ii) SQ-0018902-N (SEQ ID NO:7) and SQ-0018914-N (SEQ ID NO:12); (iii) SQ-0018902-N (SEQ ID NO:7) and SQ-0018909-N (SEQ ID NO:11); (iv) SQ-0018902-N (SEQ ID NO:7) and SQ-0018907-N (SEQ ID NO:10); (v) SQ-0018902-N (SEQ ID NO:7) and SQ-0018904-N (SEQ ID NO:9); and (vi) SQ-0018902-N (SEQ ID NO:7) and SQ-0018903-N (SEQ ID NO:8).

In some embodiments, this candidate gene is CmoCh15G012090 (cucurbitgenomics.org/v2/). Wang et al. (2022; Nat Plants. 8(12):1394-1407) reported that CmoCh15G012090 was predicted to encode a YABBY gene family transcription factor based on its phylogenetic analyses. CmoCh15G012090 encodes a protein with a zinc finger-like domain in the N terminus and a YABBY domain in the C terminus. It is also named as CmoYABBY1. Wang et al. (2022) further found that a pumpkin accession with a dominant bushy trait is identified with various B-region deletions at the 5′ UTR of CmoYABBY1 gene, which enhance the translation of YABBY1, with consequent proportional suppression of stem length in a dose-dependent manner along with the dominant bushy trait.

The present disclosure teaches that the candidate gene, CmoCh15G012090 a.k.a. CmoYABBY1, has similar deletions at the 5′ UTR in the Cucurbita plants having a determinate growth habit. In some embodiments, genetic modifications of CmoCh15G012090 are found at the 5′UTR, exons, introns, or 3′UTR from the Cucurbita plants of the present disclosure having a determinate growth habit. In some embodiments, a portion of the 5′UTR of CmoCh15G012090 is deleted. In further embodiments, at least one exon of CmoCh15G012090 is deleted. In further embodiments, at least one intron of CmoCh15G012090 is deleted. In further embodiments, a portion of the 3′UTR of CmoCh15G012090 is deleted.

In some embodiments, a genome of the Cucurbita plant of the present disclosure has genome contains at least one copy of a dominant allele for a determinate growth habit, wherein the allele localizes to chromosome 15 and is flanked on either side by one or more SNP markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO: 12), SQ-0018839 (SEQ ID NO:6), and combinations thereof. In some embodiments, the dominant allele for a determinate growth habit on chromosome 15 comprises a deleted portion of the 5′ UTR of CmoCh15G012090.

In further embodiments, a Cucurbita spp. seed, plant, part thereof, or cell thereof, have a genome that contains at least one copy of a dominant allele for a determinate or semi-determinate growth habit, wherein the allele localizes to chromosome 15 and is flanked on either side by one or more SNP markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO: 12), SQ-0018839 (SEQ ID NO:6), and combinations thereof.

In further embodiments, a Cucurbita spp. seed, plant, part thereof, or cell thereof, possess the dominant allele for a determinate or semi-determinate growth habit on chromosome 15 comprising a deleted portion of the 5′ UTR of CmoCh15G012090.

Plants of the Genus Cucurbita with Determinate Growth Habit

Once a determinate Cucurbita plant is identified from the screening as described above, it can be used for many purposes. Thus, the present disclosure provides determinate or indeterminate plants of the genus Cucurbita. In some embodiment, said plant of the genus Cucurbita is a Cucurbita moschata (C. moschata), Cucurbita pepo (C. pepo), Cucurbita okeechobeensis (C. okeechobeensis), Cucurbita pedatifolia (C. pedatifolia), Cucurbita argyrosperma (C. argyrosperma) or a Cucurbita maxima (C. maxima) plant. In some embodiments, said plant of the genus Cucurbita is a Cucurbita moschata (C. moschata), Cucurbita pepo (C. pepo) or a Cucurbita maxima (C. maxima) plant. In some embodiments, the C. moschata plant is butternut squash (e.g. C. moschata butternut type, C. moschata long neck butternut type), C. moschata flat round type, C. moschata round to oblate type, or hybrid thereof.

Indeed, as demonstrated in the example section, the inventors identified that the determinate Cucurbita plant are the plants having the determinate growth habit and harboring the genetic locus conferring said trait. They have then been able to introgress, into the indeterminate C. moschata genetic background, the sequences (i.e. quantitative trait loci (QTLs)) conferring the determinate growth habit, thus obtaining determinate C. moschata plants or semi-determinate Cucurbita plants.

The present disclosure thus provides a Cucurbita plant with a semi-determinate or determinate growth habit, wherein said plant comprises a QTL associated with said determinate growth habit on chromosome 15.

In some embodiments, said QTL on chromosome 15 is genetically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said QTL on chromosome 15 conferring the determinate growth habit is located at less than 5 cM, less than 2.5 cM, less than 1 cM, or less than 0.5 cM from markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, markers SQ-0018902-N (SEQ ID NO:7) and SQ-0018839 (SEQ ID NO:6) are physically located within about 500 kb, about 400 kb, or about 300 kb.

In some embodiments, said QTL on chromosome 15 is located in a locus encompassing the markers SQ-0018902-N (SEQ ID NO:7) and SQ-0018839 (SEQ ID NO:6). In some embodiments, said QTL on chromosome 15 is located in a locus encompassing the markers SQ-0018902-N (SEQ ID NO:7) and SQ-0018814 (SEQ ID NO:12). In some embodiments, said QTL on chromosome 15 is located in a locus encompassing the markers SQ-0018902-N (SEQ ID NO:7) and SQ-0018809 (SEQ ID NO: 11). In some embodiments, said QTL on chromosome 15 is located in a locus encompassing the markers SQ-0018902-N (SEQ ID NO:7) and SQ-0018807 (SEQ ID NO:10). In some embodiments, said QTL on chromosome 15 is located in a locus encompassing the markers SQ-0018902-N (SEQ ID NO:7) and SQ-0018804 (SEQ ID NO:9). In some embodiments, said QTL on chromosome 15 is located in a locus encompassing the markers SQ-0018902-N (SEQ ID NO:7) and SQ-0018803 (SEQ ID NO:8)

In some embodiments, said QTL on chromosome 15 is located within a chromosomal region delimited by markers SQ-0018902-N (SEQ ID NO:7) and SQ-0018839 (SEQ ID NO:6). In some embodiments, said QTL on chromosome 15 is located within a chromosomal region delimited by markers SQ-0018902-N (SEQ ID NO:7) and SQ-0018814 (SEQ ID NO:12). In some embodiments, said QTL on chromosome 15 is located within a chromosomal region delimited by markers SQ-0018902-N (SEQ ID NO:7) and SQ-0018809 (SEQ ID NO:11). In some embodiments, said QTL on chromosome 15 is located within a chromosomal region delimited by markers SQ-0018902-N (SEQ ID NO:7) and SQ-0018807 (SEQ ID NO:10). In some embodiments, said QTL on chromosome 15 is located within a chromosomal region delimited by markers SQ-0018902-N (SEQ ID NO:7) and SQ-0018804 (SEQ ID NO:9). In some embodiments, said QTL on chromosome 15 is located within a chromosomal region delimited by markers SQ-0018902-N (SEQ ID NO:7) and SQ-0018803 (SEQ ID NO:8) These markers have predictive value for the phenotype of interest, i.e. the determinate growth habit.

In some embodiments, said QTL on chromosome 15 conferring determinate growth habit according to the present disclosure is homozygously present in the genome of the plant.

In some embodiments, said QTL on chromosome 15 conferring semi-determinate growth habit according to the present disclosure is heterozygously present in the genome of the plant.

Such plant can be used to produce plants with additional desired traits by self-crossing or out-crossing.

The SNP markers associated with the determinate and indeterminate growth habits are described in Table 1.

TABLE 1
SNP markers linked to determinate growth habit, their location on the public Cucurbita
moschata genome database (cucurbitgenomics.org/organism/9; Sun H et al. (2017) Karyotype
stability and unbiased fractionation in the paleo-allotetraploid Cucurbita genomes. Molecular
Plant 10: 1293-1306) and flanking sequences. The SNP is identified in bold and in brackets.
D allele *: Determinate allele. I allele **: Indeterminate allele

| | | SNP physical | | | |
|---|---|---|---|---|---|
| | | position on | | | |
| | | Chromosome 15 | | | |
| | | of public map | | | |
| | | Cucurbita | | | |
| | | moschata | D | I | SEQ |
| SNP name | Sequence surrounding the SNP | Rifu (v 1.0) | Allele** | Allele* | ID NO |
| SQ-0018902 | TTATAATTAGTATTAGAATTAGGAATC | 8,473,188 | G/G | A/A | 1 |
| | GAATGGTGTGCCAACAAGGACACTGAC | | | | |
| | CCATACCTAATACACGACGAGTAAGAA | | | | |
| | CTTATATGTATTTTTTTTAATTGAAAT | | | | |
| | TGATTGATAAGGCTAATGGAATGAAAA | | | | |
| | GAATGAATAAAATTG[A/G]TTAAAGA | | | | |
| | GGTATATAAAGACCCACATATCATCAT | | | | |
| | GATTGTTGCCCATTTACTCAATCTTCC | | | | |
| | GTTCTCATAATTATTCCTAATAATAAT | | | | |
| | TAACCAAACTAAATGTCTAACATAATT | | | | |
| | TTATTTTCAAAATTTAGATATTTTTGT | | | | |
| | CACTAATA | | | | |
| SQ-0018903 | GTAGCCATTGTCAAATGACTCAAATTC | 8,476,073 | C/C | G/G | 2 |
| | TGTGTTTTGCTGTGACTCAAAGCATCA | | | | |
| | AAAATAATATTATTTACATTTTAATTT | | | | |
| | AATTTAATTTTTTTTATAAAGAAAAAA | | | | |
| | GTGGATACATTATTTCCCTTATGTCCT | | | | |
| | AAGCATATGGCATTA[C/G]TCCCTAA | | | | |
| | ACAGGATTAGCCCTAATAATGCAATTC | | | | |
| | TTGAACTCAAATTAGAAAGCATCAATT | | | | |
| | TTTAATCCCATTCGAGTTTTGTAGGGT | | | | |
| | AGTGACCAGATTGAGGTACTTTCACGT | | | | |
| | GTTCAACAAACTATAGCGTAACGTTGG | | | | |
| | AGTAAATT | | | | |
| SQ-0018904 | AATTTAATTTAATTTTTTTTATAAAGA | 8,476,149 | G/G | A/A | 3 |
| | AAAAAGTGGATACATTATTTCCCTTAT | | | | |
| | GTCCTAAGCATATGGCATTAGTCCCTA | | | | |
| | AACAGGATTAGCCCTAATAATGCAATT | | | | |
| | CTTGAACTCAAATTAGAAAGCATCAAT | | | | |
| | TTTTAATCCCATTCG[A/G]GTTTTGT | | | | |
| | AGGGTAGTGACCAGATTGAGGTACTTT | | | | |
| | CACGTGTTCAACAAACTATAGCGTAAC | | | | |
| | GTTGGAGTAAATTAAAAGTACATGAAT | | | | |
| | GAATCGACACTATGAATATTATTAAGA | | | | |
| | ACTTCAAACGTAAGTATGAAGTTGTTG | | | | |
| | ATGACATT | | | | |
| SQ-0018907 | GCCCGTGCTACAGCCTCTCCCCCGTTC | 8,477,012 | A/A | C/C | 4 |
| | GCCTCGGCATCAATACCGCCTCGGCAA | | | | |
| | ATGCCAGAATTTTCAACTTAGATATGA | | | | |
| | ACAAGATATGGCTATTCTAAGGTCGAA | | | | |
| | TGCGTTTCGAGGAATACGAAAGTGAAT | | | | |
| | CTCATATGAACAACA[A/C]TGTTGGT | | | | |
| | TCCACCCCTACATCACCCAAAAATAGT | | | | |
| | GAAAAGAATTCAATAAATCCTAGGAGT | | | | |
| | CTAAAAAAAAAAGTATGGAAAATGGTT | | | | |
| | ACACCGAGGAGGAATCGGCGAAAACTT | | | | |
| | GATTAAGCATATGATCGTCGTCGTTGC | | | | |
| | CGTTGGGT | | | | |
| SQ-0018909 | AATTAAATTTCATAAATCGTAATGAAA | 8,647,279 | A/A | G/G | 5 |
| | TTATACGTAGCTTCTTTTAAGTTATTT | | | | |
| | TTAGCTATGTTATATTTGGAATCGCTG | | | | |
| | TCATTCATATACTCTTTTTTTTACTCG | | | | |
| | GGTGTCACATAGCTATCCTCTTAAATC | | | | |
| | ACGCGTAATAATATC[A/G]TATTAAT | | | | |
| | TAGCTAATTATACATGATATGAGGAGA | | | | |
| | TTTGGATTTGGGGACGAATATGGGATT | | | | |
| | TTCTACCTTATTTGGAAATTTCATAAT | | | | |
| | TGATTCCAATTTAATTTGGAATAAATC | | | | |
| | TTAAACCAAATCCAAAATATAATGGAT | | | | |
| | TTTTCTTG | | | | |
| SQ-0018839 | GCCCGTGCTACAGCCTCTCCCCCGTTC | 8,477,012 | A/A | C/C | 6 |
| | GCCTCGGCATCAATACCGCCTCGGCAA | | | | |
| | ATGCCAGAATTTTCAACTTAGATATGA | | | | |
| | ACAAGATATGGCTATTCTAAGGTCGAA | | | | |
| | TGCGTTTCGAGGAATACGAAAGTGAAT | | | | |
| | CTCATATGAACAACA[C/A]TGTTGGT | | | | |
| | TCCACCCCTACATCACCCAAAAATAGT | | | | |
| | GAAAAGAATTCAATAAATCCTAGGAGT | | | | |
| | CTAAAAAAAAAAGTATGGAAAATGGTT | | | | |
| | ACACCGAGGAGGAATCGGCGAAAACTT | | | | |
| | GATTAAGCATATGATCGTCGTCGTTGC | | | | |
| | CGTTGGGT | | | | |
| SQ-0018902-N | CTCATACTAATGGAGATTGTGTCCCTC | 8,199,135 | C | T | 7 |
| | ACTTATGTACCCATGAGCTTCCTGTTA | | | | |
| | ATTAGCCAACGTGGAACTCAAACAATC | | | | |
| | CTCAACAATCTTCACATGGGACAAAGT | | | | |
| | ACACCATATAGCCTCTCCTAAGGCCTA | | | | |
| | TCTACTTCTTTGGAG[T/C]CCTTGAA | | | | |
| | CAACCTTTGTTTAATCGAAGTTCAACT | | | | |
| | CCTTTCGAGCCCTTGGAGAAAATACAC | | | | |
| | CCTCTATTCAACACGTGACTCACTAAA | | | | |
| | ACAGCTCACAACTTTCAAAGAAAAATA | | | | |
| | TTTTTACAATTGTTTCTTTTAAAAAGC | | | | |
| | CACTCAAC | | | | |
| SQ-0018903-N | TTAGAGAATAAATAATAAAAAATTAAT | 8,417,827 | A | G | 8 |
| | TATAAAATAAAATAAATATATATAATT | | | | |
| | GAGTTCGTTAGTTATATTCAAAATTTT | | | | |
| | GTTATGGTTAATTCATAAAATATCTAT | | | | |
| | TTATTAAAATTATTTTTCAAAAAAAAA | | | | |
| | TTTGTAAGAGCAGTG[G/A]GAGAATA | | | | |
| | AATAATAAAAAATAGTAATAAAATAAA | | | | |
| | ATAAAATAAATATACTGTATGTATAAC | | | | |
| | TGTATTATAAGCTAATTTTTTTTAATC | | | | |
| | AACAGTAAATTTGAATTAATTTGTTAT | | | | |
| | CAGGGGAATCTACTCACTAATTCGGTC | | | | |
| | CATAAAAT | | | | |
| SQ-0018904-N | AATATGGTCTACATTTTGTCACTGCTA | 8,426,091 | C | G | 9 |
| | CCCATACTACCTATTCTATTGTGGGTT | | | | |
| | CAAATCTATTAACTTGTTGTTACTAAC | | | | |
| | TCTATTAACTAATCCTCCTAAAGATGC | | | | |
| | TATTGTATAGGATCTAGATTCATATTA | | | | |
| | GAGTCATGACTTTAA[G/C]TTAACCA | | | | |
| | TATAAATATAATCCTCAAATGTCGAAT | | | | |
| | AAAAAAGTTATGAATCTAGAAGGTGTA | | | | |
| | GGCAAAAAGTGACTCAAGTGCGAACTC | | | | |
| | GGGTGTACTTTGTTTTGAGGGCTCTAG | | | | |
| | AGAAAGAAGTCAAGACTCAATTAAAGG | | | | |
| | GAGGTTGT | | | | |
| SQ-0018907-N | CAATCCGTAACCTACTAGTAGAGGAAG | 8,471,787 | C | G | 10 |
| | ACCCCCTAAAAATAAAAGACGTAGGGC | | | | |
| | CAACCAGAGAATCAAAAGAGGATATTT | | | | |
| | TTGTGCAGAATTTTAAAGATTTTTCTA | | | | |
| | ATATATTTAATCTGTGTCAGAAAGATT | | | | |
| | AAACTCGGAATCTTT[G/C]ATCGAAT | | | | |
| | ACATTATGTTTAACTTATTTAAAAAGG | | | | |
| | TTAGAGGTTTAAATTAATAATGATATT | | | | |
| | TACTATTTTATCTACTGAATTATGCTC | | | | |
| | AAATGGGTAAAAAAAATAAAAAAACAA | | | | |
| | ACATTTTCCACGAATCTTTAATTACTA | | | | |
| | AAGTTTTT | | | | |
| SQ-0018909-N | ACCAAAAAGTAATGTATAATAATTTTC | 8,472,832 | T | C | 11 |
| | TTTACAGAAATTTATCTTTCTTTTTTT | | | | |
| | CCTTTTCCAAAAGGTTAAATTAAGATA | | | | |
| | ATTTTTTATATTTCATAGTAAATTTAA | | | | |
| | TATTCAATTTTATCGTATAAAATAAAT | | | | |
| | AGGTGCAATAATAGG[C/T]AATTAAT | | | | |
| | AATAATAATCGAGATTTTTTTATTAAA | | | | |
| | AAATAATGCATTATATTTAGGCAATTT | | | | |
| | TTAAAAATAAATAAATTTAACAAAGAA | | | | |
| | AAAAAAAACAGGAGAGAAGCTGCAATT | | | | |
| | GACAAAGCTCACGTACATGCAAAGTCC | | | | |
| | AATGAGGA | | | | |
| SQ-0018914-N | TGTAAGCATGAACTTGTGTTGGTATGT | 8,476,501 | T | C | 12 |
| | GGAGATAATATGATCGTATGATATAAG | | | | |
| | GGATGCAAAGACATGTCAAGGTCATTT | | | | |
| | AGTAGGTAACGTGGTCATGTCGCAAGA | | | | |
| | GACGTAAGTAAGTGAATGTTAGGCTTA | | | | |
| | TTTTGTGCTAGTCGA[C/T]GAAGTGA | | | | |
| | CCATGCGGCAGAGGATGGCGTTTGTAG | | | | |
| | CAATCAAAACTGGGTGAGAGTGTGCAT | | | | |
| | GAAAACAAACTGTCAAGAATTGAGGAG | | | | |
| | TTGAGAATGTTAAAAGGGTTTGATTTG | | | | |
| | ATGGCAAAACGCTTCCCTCTCATCCTT | | | | |
| | TTTCCCTC | | | | |

SEQ ID NOs: 4 and 6 are identical and can be interchangeably used herein.

Insofar as the QTL conferring determinate growth habit can be identified by the specific alleles (i.e., D alleles) described in Table 1, a plant of the disclosure may comprise at least one of the following alleles at homozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant of the disclosure conferring a determinate trait may comprise all of the following alleles at homozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant conferring a determinate trait may also comprise a combination of at least two of the following alleles at homozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant of the disclosure may comprise any combination of the following alleles at homozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). at homozygous state.

In some embodiments, a plant of the disclosure conferring a semi-determinate growth habit may comprise at least one of the alleles described herein at heterozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant of the disclosure conferring a semi-determinate growth habit may comprise all of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at heterozygous state. In another embodiment, the plant conferring a semi-determinate growth habit may also comprise a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant conferring a semi-determinate growth habit may also comprise any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at heterozygous state.

The present disclosure thus provides Cucurbita plant comprising: a Quantitative Trait Locus (QTL) associated with a determinate growth habit. In some embodiments, said QTL is located on chromosome 15 in a locus encompassing markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said QTL is genetically linked to markers SQ-0018902-N (SEQ ID NO:7) and SQ-0018839 (SEQ ID NO:6). In other embodiments, said QTL is genetically linked to markers SQ-0018902-N (SEQ ID NO:7) and SQ-0018814 (SEQ ID NO:12). In other embodiments, said QTL is genetically linked to markers SQ-0018902-N (SEQ ID NO:7) and SQ-0018909-N (SEQ ID NO:11). In other embodiments, said QTL is genetically linked to markers SQ-0018902-N (SEQ ID NO:7) and SQ-0018807 (SEQ ID NO:10). In other embodiments, said QTL is genetically linked to markers SQ-0018902-N (SEQ ID NO:7) and SQ-0018804 (SEQ ID NO:9). In other embodiments, said QTL is genetically linked to markers SQ-0018902-N (SEQ ID NO:7) and SQ-0018803 (SEQ ID NO:8)

In some embodiments, said QTL is located within a chromosomal region delimited by markers SQ-0018902-N (SEQ ID NO:7) and SQ-0018839 (SEQ ID NO:6). In other embodiments, said QTL is located within a chromosomal region delimited by markers SQ-0018902-N (SEQ ID NO:7) and SQ-0018814 (SEQ ID NO:12). In other embodiments, said QTL is located within a chromosomal region delimited by markers SQ-0018902-N (SEQ ID NO:7) and SQ-0018909-N (SEQ ID NO:11). In other embodiments, said QTL is located within a chromosomal region delimited by markers SQ-0018902-N (SEQ ID NO:7) and SQ-0018807 (SEQ ID NO:10). In other embodiments said QTL is located within a chromosomal region delimited by markers SQ-0018902-N (SEQ ID NO:7) and SQ-0018804 (SEQ ID NO:9). In other embodiments, said QTL is located within a chromosomal region delimited by markers SQ-0018902-N (SEQ ID NO:7) and SQ-0018803 (SEQ ID NO:8). These markers have predictive value for the phenotype of interest, i.e. a determinate growth habit.

In some embodiments, said QTL is introgressed into a genus Cucurbita plant displaying an indeterminate growth trait. In some embodiments, the genus Cucurbita plant is a Cucurbita moschata (C. moschata), Cucurbita pepo (C. pepo), Cucurbita okeechobeensis (C. okeechobeensis), Cucurbita pedatifolia (C. pedatifolia), Cucurbita argyrosperma (C. argyrosperma) or a Cucurbita maxima (C. maxima) plant. In further embodiments, the genus Cucurbita is a Cucurbita moschata (C. moschata), Cucurbita pepo (C. pepo) or a Cucurbita maxima (C. maxima) plant.

In some embodiments, the Cucurbita plant has a determinate growth with a limited number of short internodes when compared to a Cucurbita plant not having said QTL associated with a determinate growth habit.

In some embodiments, said QTL is present in the genome of said Cucurbita plant, a representative sample of seed of which has been respectively deposited under NCIMB accession No. 43740.

In some embodiments, said plant is a progeny of said Cucurbita plant deposited with NCIMB accession No. 43740.

Also provided is a cell of the Cucurbita plant according to the Cucurbita plant taught herein. The cell comprises the QTL associated to a determinate growth.

Provided is a plant part obtained from a Cucurbita plant according to the Cucurbita plant taught herein. In some embodiments, said plant part is a seed, a fruit, a reproductive material, roots, flowers, a rootstock or a scion. The plant part comprises the QTL associated to a determinate growth.

Further provided is a seed of a Cucurbita plant according to the Cucurbita plant taught herein. The seed comprises the QTL associated to a determinate growth.

Further provided is a fruit of a Cucurbita plant according to the Cucurbita plant taught herein. The fruit comprises the QTL associated to a determinate growth.

Plants grown from the deposited seeds are indeed homozygously determinate, i.e., they bear in their genome the QTL associated with determinate growth habit on chromosome 15 as defined here above at homozygous state. They can be used to transfer this QTL in another background by any suitable methods, such as by crossing and selfing and/or backcrossing. A progeny of a plant obtained from the deposited seed can be identified by one skilled in the art, for example by using the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6), and any other markers within a chromosomal region delimited by markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

The present disclosure also provides a plant's part derived from a plant of the genus Cucurbita with determinate growth habit. In some embodiments, said plant's part derives from a Cucurbita plant according to the disclosure, i.e. said plant's part comprises the QTL on chromosome 15 as defined above conferring the determinate growth habit.

In some embodiments, a part of plant is a plant cell. The disclosure thus provides an isolated cell of a Cucurbita plant according to the disclosure, i.e. a cell that comprises the QTL on chromosome 15 as defined above conferring the determinate growth habit.

In some embodiments, the alleles conferring the determinate growth habit are as described in Table 1. In some embodiments, the plant, plant part, or plant cell, which possess a determinate growth habit according to the disclosure thus may comprise at least one of the following alleles at homozygous or heterozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In some embodiments, the plant part according to the disclosure may comprise all of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous or heterozygous state. In some embodiments, the plant part according to the disclosure may also comprise a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous or heterozygous state. In some embodiments, the plant part according to the disclosure may comprise any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous or heterozygous state.

In some embodiments, the QTL or allele(s) as described here above are chosen from those present in the genome of a plant corresponding to the deposited C. moschata plant (NCIMB accession no. 43740).

In some embodiments, the QTL or allele(s) as described here above are as found in the genome of a plant corresponding to the deposited C. moschata plant (NCIMB accession no. 43740).

A plant cell of the disclosure may have the capacity to be regenerated into a whole plant, said plant having a commercially acceptable fruit quality.

Alternatively, the disclosure is also directed to plant cells which are not regenerable, and thus not capable of giving rise to a whole plant.

According to another embodiment, the plant part is any other part of a plant according to the disclosure; it may be in particular seeds, reproductive material, roots, flowers, fruits, rootstock or scion. Such a part comprises a cell as defined above.

The present disclosure also provides seed derived from a plant population, plant, plant part, plant tissue or plant cell of the genus Cucurbita, wherein said seed can give rise to a plant of the genus Cucurbita that is also determinate or semi-determinate. In some embodiments, said seed derives from a plant population, plant, plant part, plant tissue or plant cell of a Cucurbita plant according to the disclosure, i.e. said seed is determinate in growth habit due to the QTL on chromosome 15 as defined here above conferring said determinacy. In some embodiments, the plant obtained from said seed is determinate due to the presence of said QTL on chromosome 15 at homozygous state as defined here above conferring said determinacy. In some embodiments, the plant obtained from said seed is identified as being determinate due to the presence of at least one of the following alleles at homozygous state on chromosome 15: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant obtained from said seed is identified as being determinate due to the presence of all the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous state on chromosome 15. In another embodiment, the plant obtained from said seed is identified as being determinate due to the presence of a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous state. In another embodiment, the plant obtained from said seed is identified as being determinate due to the presence of any combination of the following alleles allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous state.

In some embodiments, the plant obtained from said seed is semi-determinate due to the presence of said QTL on chromosome 15 at heterozygous state as defined here above conferring said determinacy. In some embodiments, the plant obtained from said seed is identified as being semi-determinate due to the presence of at least one of the following alleles at heterozygous state on chromosome 15: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant obtained from said seed is identified as being semi-determinate due to the presence of all of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at heterozygous state on chromosome 15. In another embodiment, the plant obtained from said seed is identified as being semi-determinate due to the presence of a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at heterozygous state. In another embodiment, the plant obtained from said seed is identified as being semi-determinate due to the presence of a combination of any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at heterozygous state.

In some embodiments, said seeds are the deposited seeds of C. moschata with determinate growth habit (NCIMB accession no. 43740). These seeds contain homozygously the QTL on chromosome 15 conferring the determinate growth habit as defined above; they are however distinct on other phenotypic traits such that they do not give rise to a plant variety.

The present disclosure also provides a tissue culture of a plant population, plant, plant part, plant tissue or plant cell of the genus Cucurbita, wherein said tissue culture retains determinate growth habit. In some embodiments, said tissue culture is a tissue culture of a plant population, plant, plant part, plant tissue or plant cell of a Cucurbita plant according to the disclosure, i.e. a tissue culture that comprises the QTL on chromosome 15 as defined above conferring the determinate growth habit.

The present disclosure also provides a progeny derived from the plant of the genus Cucurbita as described above, whether produced sexually or asexually, wherein said progeny retains determinate or semi-determinate growth habit. In some embodiments, said progeny derives from a plant a Cucurbita plant according to the disclosure, i.e. said progeny bears in its genome the QTL associated to determinate growth habit on chromosome 15 as defined here above at homozygous state. In some embodiments, said progeny bears in its genome the QTL associated to determinate growth habit on chromosome 15 as defined here above at heterozygous state.

Thus, the present disclosure provides methods of isolating a nucleic acid sequence conferring the entire determinate growth habit from a determinate plant of the genus Cucurbita, plant tissue, or plant cell, comprising:
a) crossing the determinate plant of the genus Cucurbita as a donor with an indeterminate, or partially indeterminate plant of the genus Cucurbita to get offspring plants as a mapping population,
b) determining the determinacy in said offspring plants, and
c) cloning the nucleic acid sequence. For example, by map-based cloning or association mapping.

One skilled in the art will know how to choose a suitable plant for crossing, and how to clone a nucleic acid sequence by map-based cloning (see, Varshney and Tuberisa, Genomics-assisted crop improvement: Genomics application in crops, Volume 2 of Genomics-assisted Crop Improvement, 2008, Springer, Loze and Wenzel, Molecular marker systems in plant breeding and crop improvement, 2007, Springer, ISBN. 3540740066 9783540740063; Kang, Quantitative genetics, genomics, and plant breeding, 2002, CABI, ISBN 0851996019, 9780851996011, each of which is incorporated herein by reference in its entirety). Such cloned nucleic acid sequence can be transformed into an indeterminate plant to make it become determinate. Methods of plant transformation is well-known in the art, and described separately below. Alternatively, genome fragment comprising said nucleic acid from a donor plant of the genus Cucurbita which is determinate can be transferred to a recipient indeterminate plant of the genus Cucurbita through any transferring and/or breeding method described separately below.

QTL (quantitative trait locus) mapping can be applied to determine the region of the donor plant's genome conferring the determinate growth habit.

Thus, the present disclosure provides methods of detecting a QTL associated with the determinate growth habit in a donor plant of the genus Cucurbita, comprising:
a) crossing the determinate plant of the genus Cucurbita as a donor with a suitable indeterminate plant of the genus Cucurbita to produce offspring plants,
b) quantitatively determining the determinacy in said one or more offspring plants,
c) establishing a genetic linkage map that links the observed resistance to the presence of chromosomal markers of said donor plant in said one or more offspring plants, and
d) assigning to a QTL the contiguous markers on said map that are linked to determinate growth habit.

Inheritance of quantitative traits or polygenic inheritance refers to the inheritance of a phenotypic characteristic that varies in degree and can be attributed to the interactions between two or more genes and their environment. Though not necessarily genes themselves, quantitative trait loci (QTLs) are stretches of DNA that are closely linked to the genes that underlie the trait in question. QTLs can be molecularly identified to help map regions of the genome that contain genes involved in specifying a quantitative trait. This can be an early step in identifying and sequencing these genes.

Typically, QTLs underlie continuous traits (those traits that vary continuously, e.g. level of determinacy) as opposed to discrete traits (traits that have two or several character values, e.g. smooth vs. wrinkled peas used by Mendel in his experiments). Moreover, a single phenotypic trait is usually determined by many genes. Consequently, many QTLs are associated with a single trait.

A quantitative trait locus (QTL) is a region of DNA that is associated with a particular phenotypic trait—these QTLs are often found on different chromosomes. Knowing the number of QTLs that explains variation in the phenotypic trait tells about the genetic architecture of a trait. It may tell that determinate plants of the present disclosure are controlled by many genes of small effect, or by a few genes of large effect.

Another use of QTLs is to identify candidate genes underlying a trait. Once a region of DNA is identified as contributing to a phenotype, it can be sequenced. The DNA sequence of any genes in this region can then be compared to a database of DNA for genes whose function is already known.

In a recent development, classical QTL analyses are combined with gene expression profiling i.e. by DNA microarrays. Such expression QTLs (e-QTLs) describe cis- and trans-controlling elements for the expression of often disease-associated genes. Observed epistatic effects have been found beneficial to identify the gene responsible by a cross-validation of genes within the interacting loci with metabolic pathway- and scientific literature databases.

QTL mapping is the statistical study of the alleles that occur in a locus and the phenotypes (physical forms or traits) that they produce (see, Meksem and Kahl, The handbook of plant genome mapping: genetic and physical mapping. 2005, Wiley-VCH, ISBN 3527311165, 9783527311163). Because most traits of interest are governed by more than one gene, defining and studying the entire locus of genes related to a trait gives hope of understanding what effect the genotype of an individual might have in the real world.

Statistical analysis is required to demonstrate that different genes interact with one another and to determine whether they produce a significant effect on the phenotype. QTLs identify a particular region of the genome as containing a gene that is associated with the trait being assayed or measured. They are shown as intervals across a chromosome, where the probability of association is plotted for each marker used in the mapping experiment.

To begin, a set of genetic markers must be developed for the species in question. A marker is an identifiable region of variable DNA. Biologists are interested in understanding the genetic basis of phenotypes (physical traits). The aim is to find a marker that is significantly more likely to co-occur with the trait than expected by chance, that is, a marker that has a statistical association with the trait. Ideally, they would be able to find the specific gene or genes in question, but this is a long and difficult undertaking. Instead, they can more readily find regions of DNA that are very close to the genes in question. When a QTL is found, it is often not the actual gene underlying the phenotypic trait, but rather a region of DNA that is closely linked with the gene.

For organisms whose genomes are known, one might now try to exclude genes in the identified region whose function is known with some certainty not to be connected with the trait in question. If the genome is not available, it may be an option to sequence the identified region and determine the putative functions of genes by their similarity to genes with known function, usually in other genomes. This can be done using BLAST, an online tool that allows users to enter a primary sequence and search for similar sequences within the BLAST database of genes from various organisms.

Another interest of statistical geneticists using QTL mapping is to determine the complexity of the genetic architecture underlying a phenotypic trait. For example, they may be interested in knowing whether a phenotype is shaped by many independent loci, or by a few loci, and do those loci interact. This can provide information on how the phenotype may be evolving.

Molecular markers are used for the visualization of differences in nucleic acid sequences. This visualization is possible due to DNA-DNA hybridization techniques (RFLP) and/or due to techniques using the polymerase chain reaction (e.g. STS, microsatellites, AFLP, SNP). All differences between two parental genotypes will segregate in a mapping population based on the cross of these parental genotypes. The segregation of the different markers may be compared and recombination frequencies can be calculated. The recombination frequencies of molecular markers on different chromosomes is generally 50%. Between molecular markers located on the same chromosome the recombination frequency depends on the distance between the markers. A low recombination frequency corresponds to a low distance between markers on a chromosome. Comparing all recombination frequencies will result in the most logical order of the molecular markers on the chromosomes. This most logical order can be depicted in a linkage map (Paterson, 1996). A group of adjacent or contiguous markers on the linkage map that is associated to a reduced disease incidence and/or a reduced lesion growth rate pinpoints the position of a QTL.

Using QTL mapping analysis, the inventors have been able to identify one QTL conferring the determinate growth habit on chromosome 15. Said QTL on chromosome 15 confers determinate growth habit when present at homozygous state. Said QTL on chromosome 15 confers semi-determinate growth habit when present at heterozygous state.

In some embodiments, said QTL on chromosome 15 is genetically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said QTL on chromosome 15 conferring the determinate growth habit is located at less than 5 cM, less than 2.5 cM, less than 1 cM, or less than 0.5 cM from markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and/or SQ-0018839 (SEQ ID NO:6).

In some embodiments, said QTL on chromosome 15 is located in a locus encompassing the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and/or SQ-0018839 (SEQ ID NO:6).

In some embodiments, said QTL on chromosome 15 is located within a chromosomal region delimited by markers SQ-0018902-N (SEQ ID NO:7), SQ-0018839 (SEQ ID NO:6), both of which are physically located within about 500 kb, about 400 kb, or about 300 kb. These markers have predictive value for the phenotype of interest, i.e. the determinate growth habit.

The nucleic acid sequence of the QTL of the present disclosure may be determined by methods known to the skilled person. For instance, a nucleic acid sequence comprising said QTL or a determinacy-conferring part thereof may be isolated from a determinate donor plant of the genus Cucurbita by fragmenting the genome of said plant and selecting those fragments harboring one or more markers indicative of said QTL. Subsequently, or alternatively, the marker sequences (or parts thereof) indicative of said QTL may be used as (PCR) amplification primers, in order to amplify a nucleic acid sequence comprising said QTL from a genomic nucleic acid sample or a genome fragment obtained from said plant. The amplified sequence may then be purified in order to obtain the isolated QTL. The nucleotide sequence of the QTL, and/or of any additional markers comprised therein, may then be obtained by standard sequencing methods.

Such QTL associated with the determinate growth habit in a donor plant of the genus Cucurbita can be transformed into an indeterminate plant of the genus Cucurbita to make it become determinate in growth habit. Methods of plant transformation is well-known in the art, and described separately below. Alternatively, genome fragment comprising said QTL from a donor plant of the genus Cucurbita which is determinate can be transferred to a indeterminate recipient plant of the genus Cucurbita through any transferring and/or breeding method described separately below.

In one embodiment, an advanced backcross QTL analysis (AB-QTL) is used to discover the nucleotide sequence or the QTLs responsible for the determinacy or indeterminacy of a plant. Such method was proposed by Tanksley and Nelson in 1996 (Tanksley and Nelson, 1996, Advanced backcross QTL analysis: a method for simultaneous discovery and transfer of valuable QTL from un-adapted germplasm into elite breeding lines. Theor Appl Genet. 92:191-203) as a new breeding method that integrates the process of QTL discovery with variety development, by simultaneously identifying and transferring useful QTL alleles from un-adapted (e.g., land races, wild species) to elite germplasm, thus broadening the genetic diversity available for breeding. AB-QTL strategy was initially developed and tested in tomato, and has been adapted for use in other crops include rice, maize, wheat, pepper, barley, and bean. Once favorable QTL alleles are detected, only a few additional marker-assisted generations are required to generate near isogenic lines (NILs) or introgression lines (ILs) that can be field tested in order to confirm the QTL effect and subsequently used for variety development.

Isogenic lines in which favorable QTL alleles have been fixed can be generated by systematic backcrossing and introgressing of marker-defined donor segments in the recurrent parent background. These isogenic lines are referred as near isogenic lines (NILs), introgression lines (ILs), backcross inbred lines (BILs), backcross recombinant inbred lines (BCRIL), recombinant chromosome substitution lines (RCSLs), chromosome segment substitution lines (CSSLs), and stepped aligned inbred recombinant strains (STAIRSs). An introgression line in plant molecular biology is a line of a crop species that contains genetic material derived from a similar species. ILs represent NILs with relatively large average introgression length, while BILs and BCRILs are backcross populations generally containing multiple donor introgressions per line. As used herein, the term “introgression lines or ILs” refers to plant lines containing a single marker defined homozygous donor segment, and the term “pre-ILs” refers to lines which still contain multiple homozygous and/or heterozygous donor segments.

To enhance the rate of progress of introgression breeding, a genetic infrastructure of exotic libraries can be developed. Such an exotic library comprises of a set of introgression lines, each of which has a single, possibly homozygous, marker-defined chromosomal segment that originates from a donor exotic parent, in an otherwise homogenous elite genetic background, so that the entire donor genome would be represented in a set of introgression lines. A collection of such introgression lines is referred as libraries of introgression lines or IL libraries (ILLs). The lines of an ILL covers usually the complete genome of the donor, or the part of interest.

Introgression lines allow the study of quantitative trait loci, but also the creation of new varieties by introducing exotic traits. High resolution mapping of QTL using ILLs enable breeders to assess whether the effect on the phenotype is due to a single QTL or to several tightly linked QTL affecting the same trait. In addition, sub-ILs can be developed to discover molecular markers which are more tightly linked to the QTL of interest, which can be used for marker-assisted breeding (MAB). Multiple introgression lines can be developed when the introgression of a single QTL is not sufficient to result in a substantial improvement in agriculturally important traits (Gur and Zamir, Unused natural variation can lift yield barriers in plant breeding, 2004, PLoS Biol.; 2(10):e245).

In one embodiment, when it is not determined that which parts of the donor plant's genome confer the determinate growth habit, complete chromosomes of the donor plant are transferred. For example, the determinate plant of the genus Cucurbita can serve as a male or female parent in a cross pollination to produce determinate offspring plants, wherein by receiving the genomic material form the determinate donor plant, the offspring plants are determinate in growth habit.

### Methods of Producing Cucurbita Plants Having a Semi-Determinate or Determinate Growth Habit

Any Cucurbita plant bearing the QTL associated with the determinate growth habit on chromosome 15 as defined above can be used to produce more Cucurbita plants, especially, but not limited to, C. moschata, C. pepo, or C. maxima plants that are determinate in growth habit through plant breeding methods well known to those skilled in the art.

In one embodiment, a determinate plant of the genus Cucurbita is used as a donor plant of genetic material which can be transferred to produce a recipient plant which has the transferred genetic material and is also determinate in growth habit. Any suitable method known in the art can be applied to transfer genetic material from a donor plant to a recipient plant. In most cases, such genetic material is genomic material.

In one embodiment, the genome of the determinate plants of the present disclosure is transferred into a recipient plant. This can be done by crossing the determinate plants to a recipient plant to create a F1 plant. In some embodiments, the F1 plant can have the semi-determinate growth habit. In some embodiments, the F1 plant can be further selfed and selected for one, two, three, four, or more generations to give determinate plants.

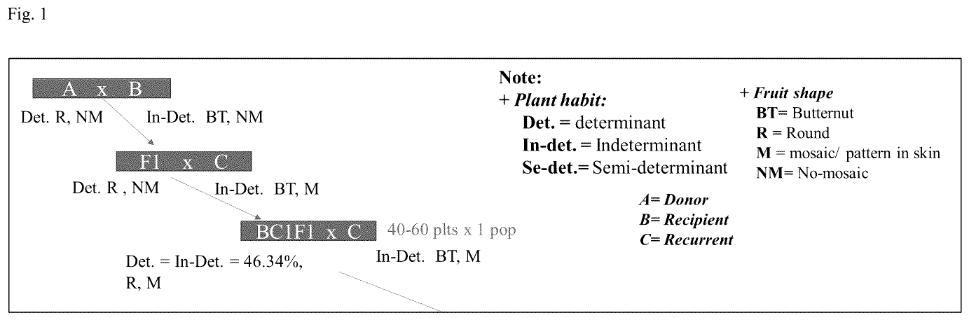

In another embodiment, at least the determinacy-conferring parts of the donor plant's genome, i.e. the QTL on chromosome 15 as defined above are transferred. This can be done by crossing the determinant plants to a recipient plant to create a F1 plant, followed optionally with one or more backcrosses to one of the parent plants to give determinate plants with the desired genetic background, as presented in FIG. 1. The progeny resulting from the backcrosses can be further selfed to give determinate plants.

According to the genetic analysis done by the inventors, the nature of the growth determinacy of the present disclosure is codominant or incompletely dominant considering the overall plant architecture. Still according to the genetic analysis done by the inventors the nature of the growth determinacy of the present disclosure is moreover (i) codominant or incompletely dominant considering the plant internode length, and/or (ii) dominant considering the terminated apex (i.e. determinate growth).

In some embodiments, said QTL conferring the determinate growth habit is located on chromosome 15 and is genetically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO: 11), SQ-0018914-N (SEQ ID NO:12), and SQ-0018839 (SEQ ID NO:6). In some embodiments, said QTL on chromosome 15 conferring the determinate growth habit is located at less than 5 cM, less than 2.5 cM, less than 1 cM, or less than 0.5 cM from markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said QTL conferring the determinate growth habit is located on chromosome 15 and is physically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO: 11), SQ-0018914-N (SEQ ID NO:12), and SQ-0018839 (SEQ ID NO:6), wherein the markers are physically located within about 500 kb, about 400 kb, about 300 kb, about 200 kb, about 100 kb, or about 50 kb. In some embodiments, said QTL on chromosome 15 conferring the determinate growth habit is located at less than 300 kb, less than 250 kb, less than 200 kb, less than 150 kb, less than 100 kb, or less than 50 kb, less than 40 kb, less than 30 kb between two markers selected from SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO: 11), SQ-0018914-N (SEQ ID NO:12), and SQ-0018839 (SEQ ID NO:6).

In some embodiments, said QTL on chromosome 15 conferring the determinate growth habit is physically located at less than 300 kb, less than 250 kb, less than 200 kb, less than 150 kb, less than 100 kb, less than 50 kb, less than 40 kb, or less than 30 kb between markers SQ-0018902-N (SEQ ID NO:7) and SQ-0018839 (SEQ ID NO:6).

In some embodiments, said QTL on chromosome 15 conferring the determinate growth habit is physically located at less than 150 kb, less than 100 kb, less than 50 kb, less than 40 kb, or less than 30 kb between markers SQ-0018902-N (SEQ ID NO:7) and SQ-0018914-N (SEQ ID NO:12).

In some embodiments, said QTL on chromosome 15 conferring the determinate growth habit is physically located at less than 150 kb, less than 100 kb, less than 50 kb, less than 40 kb, or less than 30 kb between markers SQ-0018902-N (SEQ ID NO:7) and SQ-0018909-N (SEQ ID NO:11).

In some embodiments, said QTL on chromosome 15 conferring the determinate growth habit is physically located at less than 150 kb, less than 100 kb, less than 50 kb, less than 40 kb, or less than 30 kb between markers SQ-0018902-N (SEQ ID NO:7) and SQ-0018907-N (SEQ ID NO:10).

In some embodiments, said QTL on chromosome 15 conferring the determinate growth habit is physically located at less than 150 kb, less than 100 kb, less than 50 kb, less than 40 kb, or less than 30 kb between markers SQ-0018902-N (SEQ ID NO:7) and SQ-0018904-N (SEQ ID NO:9).

In some embodiments, said QTL on chromosome 15 conferring the determinate growth habit is physically located at less than 150 kb, less than 100 kb, less than 50 kb, less than 40 kb, or less than 30 kb between markers SQ-0018902-N (SEQ ID NO:7) and SQ-0018903-N (SEQ ID NO:8).

Thus, in some embodiments, the genome segment comprising said QTL on chromosome 15 can be transferred to a recipient line though breeding methods. As also described elsewhere herein, molecular marker assisted selection can be used to facilitate the breeding.

The disclosure thus concerns method for the production of semi-determinate or determinate Cucurbita plants, especially commercial plant.

The present disclosure is indeed also directed to transferring the QTL conferring the determinate growth habit to other Cucurbita varieties, or other species and is useful for producing new types and varieties of semi-determinate or determinate Cucurbita plants.

A method or process for the production of a plant having these features may comprise the following steps:
a) Crossing a first plant bearing the QTL associated with determinate growth habit on chromosome 15 as defined above and a second Cucurbita plant as a recipient plant, in which the desired phenotype is to be imported or improved; preferably such the second Cucurbita plant is indeterminate in growth habit;
b) Selecting a plant in the progeny thus obtained bearing the QTL conferring determinate growth habit when present homozygously or semi-determinate plants when present heterozygously,
c) Optionally, self-pollinating one or several times the plant obtained at step b) and selecting a determinate plant in the progeny thus obtained;

wherein markers are used in steps b) and c), for selecting plants bearing QTL conferring determinate growth habit and/or determinate plants when present homozygously or selecting plants bearing QTL conferring semi-determinate growth habit and/or semi-determinate plants when present heterozygously. The markers are one or more of the markers of the disclosure, i.e. one or more of the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6). According to an embodiment, the selection is at least made on the basis of the alleles of markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6). The selection can also be made on the detection of the alleles of all the markers of the disclosure.

The plant, which is selected at the selection step disclosed above, is selected on the presence of at least one of the following alleles on chromosome 15: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant, which is selected at the selection step disclosed above, is selected on the presence of all the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In still another embodiment, the plant, which is selected at the selection step disclosed above, is selected on the presence of a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In still another embodiment, the plant, which is selected at the selection step disclosed above, is selected on the presence of any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6).

In order to identify plants bearing homozygously or heterozygously the QTL responsible for the determinate growth habit, the presence of the allele linked to the determinacy is to be detected in combination with the absence of the allele linked to the indeterminate parent.

In order to identify plants bearing heterozygously the introgressed sequences, the sole presence of the allele linked to the determinacy is to be detected.

The method used for allele detection can be based on any technique known by the one skilled in the art allowing the distinction between two different allele of a marker, on a specific chromosome.

A method or process as defined above may advantageously comprises backcrossing steps, preferably after step b) or c), in order to obtain plants having all the characterizing features of Cucurbita plants as a recipient. Consequently, a method or process for the production of a plant having these features may also comprise the following additional steps:
d) Backcrossing the determinate plant selected in step b) or c) with an indeterminate Cucurbita plant;
e) Selecting a plant in the progeny bearing QTL conferring determinate growth habit when present homozygously or semi-determinate plants when present heterozygously,
f) Optionally, self-pollinating the plant obtained at step e), and
g) Selecting a determinate plant in growth habit when present homozygously or selecting a semi-determinate plant when present heterozygously.

The plant used in step a) can be a plant grown from the deposited seeds NCIMB No. 43740); it may alternatively be any plant according to the disclosure, i.e. homozygously determinate in growth habit.

Alternatively, the method or process may comprise the following steps:
a1) Crossing a plant corresponding to the deposited seeds (NCIMB No. 43740) and a Cucurbita plant, in which the desired phenotype is to be imported or improved, thus generating the F1 population;
a2) Optionally, selfing the F1 population to create F2 population;
b) Selecting determinate individuals in the progeny thus obtained bearing the QTL conferring determinate growth habit when present homozygously or semi-determinate plants when present heterozygously;
c) Optionally self-pollinating one or several times the determinate or semi-determinate plant obtained at step b) and selecting a determinate plant in the progeny thus obtained;
d) Backcrossing the determinate progeny plants selected in step b) or c) with Cucurbita plant,
e) Selecting in the progeny a plant bearing an QTL linked to the desired phenotype or a plant being determinate in growth habit when present homozygously or semi-determinate plants when present heterozygously,
f) Optionally, self-pollinating the plant obtained at step e) one or several times, and
g) Selecting a determinate plant when present homozygously or selecting a semi-determinate plant when present heterozygously.

The Cucurbita plant of steps a) or al) and d) is preferably an indeterminate plant or a less determinate plant than the determinate plants of the disclosure.

The plant selected at step b), c), e) or g) of the preceding methods may be a commercial plant, especially a plant having a bushy type with short internode, early yield and homogeneous fruit color and shape.

Steps d), e) and/or f) may be repeated twice or three times or more, not necessarily with the same Cucurbita plant. Said Cucurbita plant is preferably a breeding line. This plant is preferably an elite line, used in order to introduce commercially desired traits or desired horticultural traits.

The self-pollination/crossing and backcrossing steps may be carried out in any order and can be intercalated, for example a backcross can be carried out before and after one or several self-pollinations/crossings, and self-pollinations/crossings can be envisaged before and after one or several backcrosses.

Moreover, the methods of the disclosure are advantageously carried out by using markers as described above for one or more of the selection steps for selecting plants bearing the QTL conferring the determinate growth habit, or for selecting plants having the phenotype of interest. The markers are one or more of the markers of the disclosure, i.e. one or more of the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

According to a preferred embodiment, the selection is at least made on the basis of the allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). The selection can also be made on the detection of the alleles of all the markers of the disclosure.

The plant selected at any one of steps b), e) and/or g) is preferably selected on the presence of at least one of the following alleles on chromosome 15: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In another embodiment, the plant, which is selected at the selection step disclosed above, is selected on the presence of all the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In still another embodiment, the plant, which is selected at the selection step disclosed above, is selected on the presence of a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In still another embodiment, the plant, which is selected at the selection step disclosed above, is selected on the presence of a combination of any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6).

The method used for allele detection can be based on any technique allowing the distinction between two different alleles of a marker, on a specific chromosome locus.

The present disclosure also concerns any breeding scheme involving as first step crossing a plant grown from one of the deposited seeds (NCIMB No. 43740).

The present disclosure also concerns a plant obtained or obtainable by one of the methods described above. Such a plant is indeed a Cucurbita (e.g. a C. moschata, C. pepo, or C. maxima) plant having the desired phenotype according to the disclosure, i.e. a plant that is semi-determinate or determinate in growth habit.

The disclosure also provides a method for producing a hybrid Cucurbita seed comprising crossing a first cultivar plant parent with a second cultivar plant parent and harvesting the resultant hybrid Cucurbita seed, wherein both parents are cultivars containing the QTL as defined in the disclosure in the homozygous or heterozygous state. The hybrid seeds, plant and parts thereof produced by such method are also part of the disclosure.

In a method for producing a semi-determinate or determinate plant of the genus Cucurbita, protoplast fusion can also be used for the transfer of determinacy-conferring genomic material from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained with plant species that cannot be interbred in nature, is tissue cultured into a hybrid plant exhibiting the desirable combination of traits. More specifically, a first protoplast can be obtained from a plant line of the genus Cucurbita that is determinate. For example, a protoplast from a determinate squash line may be used. A second protoplast can be obtained from an indeterminate second plant line, optionally from another plant species or variety, such as from the same plant species or variety, that comprises commercially desirable characteristics, such as, but not limited to disease resistance, insect resistance, valuable fruit characteristics, etc. The protoplasts are then fused using traditional protoplast fusion procedures, which are known in the art to produce the cross.

Alternatively, embryo rescue may be employed in the transfer of determinacy-conferring genomic material from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (see Pierik, 1999, In vitro culture of higher plants, Springer, ISBN 079235267x, 9780792352679, which is incorporated herein by reference in its entirety).

In addition, in one embodiment, a method for producing a determinate plant of the genus Cucurbita comprises grafting an indeterminate recipient plant of the genus Cucurbita onto rootstocks of determinate plants, which is proved to be an effective methodology developed for intensive cultivation in the Far East (Lee and Oda, 2003, Grafting of herbaceous vegetable and ornamental crops, Hort. Rev. 28:61-124).

In some embodiments, the recipient plant is a squash plant, such as a C. moschata, C. pepo, or C. maxima plant, or any other cucurbit plant that can hybridize with the determinate plant of the disclosure.

In one embodiment, the recipient plant is an elite line having one or more certain agronomically important traits. As used herein, “agronomically important traits” include any phenotype in a plant or plant part that is useful or advantageous for human use. Examples of agronomically important traits include but are not limited to those that result in increased biomass production, production of specific biofuels, increased food production, improved food quality, etc. Additional examples of agronomically important traits includes pest resistance, vigor, development time (time to harvest), enhanced nutrient content, novel growth patterns, flavors or colors, salt, heat, drought and cold tolerance, and the like. Agronomically important traits do not include selectable marker genes (e.g., genes encoding herbicide or antibiotic resistance used only to facilitate detection or selection of transformed cells), hormone biosynthesis genes leading to the production of a plant hormone (e.g., auxins, gibberellins, cytokinins, abscisic acid and ethylene that are used only for selection), or reporter genes (e.g. luciferase, β-glucuronidase, chloramphenicol acetyl transferase (CAT, etc.).

Other agronomically important traits include resistance to biotic and/or abiotic stresses. As used herein, the phrase “biotic stress” or “biotic pressure” refers to a situation where damage is done to plants by other living organisms, such as bacteria, viruses, fungi, parasites, insects, weeds, animals and human. As used herein, the phrase “abiotic stress” or “abiotic pressure” refers to the negative impact of non-living factors on plants in a specific environment. The non-living variable must influence the environment beyond its normal range of variation to adversely affect the population performance or individual physiology of plants in a significant way. Non-limiting examples of stressors are high winds, extreme temperatures, drought, flood, and other natural disasters, such as tornados and wildfires. For example, the plant lines developed using the genetic materials and methods of the present disclosure can also include determinate growth habit due to one or more different loci other than the QTL on chromosome 15 as defined here above.

A list of popular North America squash cultivars with various agronomically important traits can be found in the Cucurbit Breeding database of North Carolina State University (Wessel-Beaver et al., Vegetable Cultivar Descriptions for North America, Squash, Retrieved on Apr. 21, 2010, incorporated by reference in its entirety).

The recipient line will have one or more preferred Cucurbit traits. These traits include, but are not limited to, resistance/tolerance to pathogens, such as to Bacterial wilt (Erwinia tracheiphila), Alternaria leaf blight (Alternaria cucumerina), Downy mildew (Pseudoperonospora cubensis), Powdery mildew (Erysiphe spp. or Sphaerotheca spp.), Squash Mosaic Virus, Zucchini Yellow Mosaic Virus, Phytophthora blight (Phytophthora capsici), Tomato Leaf Curl New Delhi Virus (ToLCNDV); resistance/tolerance to insects such as to cucumber beetles, squash beetles, spider mites, aphids, squash vine borers, pickleworms, worms, white fly, root-knot nematode; specific flower-fruit related traits, such as traits related to abscission, bitterness, blossom scar, fruit skin pattern, flesh color, flesh thickness, fruit diameter, fruit length, fruit rib, fruit shape, seed cavity color, fruit skin texture, spine color and fruit weight; specific type of plant growth habit; certain specific morphological traits, such as size/type of the blossom end fruit shape, size/type of cavity diameter, size/type of the blossom scar, ease of peduncle separation from fruit, ease of seed separation from flesh, external aroma, flesh color intensity, flesh flavor, flesh moisture, flower color, fruit skin corking, fruit skin glossiness, fruit splitting, fruit stem color, fruit stripes on blossom end, fruit volume, fruit width, internal aroma, internal color of skin, internode length, leaf color, leaf lobes, leaf shape, leaf size, number of fruits harvested per plant, number of seeds per fruit, seed coat color, seed shape, seed size, skin hardness of fruit), or certain preferred phenological traits, such as a desired time of maturity (based on accumulated heat units, days after planting, and/or day length), desired production related traits (e.g., 100 seed weight, flesh dry matter percent, fruit storage ability, fruit weight), and/or desired stress related traits (e.g., tolerance to drought, tolerance to salt, tolerance to low and high temperatures).

As mentioned above, the determinacy in the determinate plant provided by the present disclosure is likely due to a QTL in the genome based on genetic analysis. In some embodiments, said QTL conferring the determinate growth habit is located on chromosome 15 and is genetically linked to the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6) as defined here above. In some embodiments, said QTL conferring the determinate growth habit is located on chromosome 15 in a locus encompassing the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6). In some embodiments, said QTL conferring the determinacy is located on chromosome 15 within a chromosomal region delimited by markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

One skilled in the art will know how to clone said QTL on chromosome 15 using the determinate plants of the present disclosure. For example, one skilled in the art will be able to choose a suitable plant for crossing, generate a mapping population, and isolate polynucleotide of the gene responsible for the determinacy located in said QTL on chromosome 15 by map-based cloning or any other suitable methods (see, Varshney and Tuberisa, Genomics-assisted crop improvement: Genomics application in crops, Volume 2 of Genomics-assisted Crop Improvement, 2008, Springer, Loze and Wenzel, Molecular marker systems in plant breeding and crop improvement, 2007, Springer, ISBN. 3540740066 9783540740063; Kang, Quantitative genetics, genomics, and plant breeding, 2002, CABI, ISBN 0851996019, 9780851996011, each of which is incorporated herein by reference in its entirety). Such isolated polynucleotide sequence can be transferred into a recipient indeterminate plant through any breeding method described separately below, to make a new line that is determinate.

The isolated polynucleotide of gene responsible for the determinate growth habit located in said QTL on chromosome 15 can be used in many aspects. In one embodiment, the nucleic acid sequence of said isolated gene, or any function variant thereof, can be expressed in other plant species that are indeterminate in growth habit, and wherein said species cannot hybridize with Cucurbits plants of the present disclosure. For example, said species is other Cucurbitaceae species, such as squash, pumpkin, butternut squash, melon, cucumber, watermelon. In other embodiment, said isolated gene, or fragment thereof can be used as probe to identify and/or isolate homologous genes in other plants.

Molecular Markers Closely Linked to QTL Associated with Determinate Growth Habit on Chromosome 15

The inventors of the present disclosure also provide molecular markers that are tightly linked to the locus conferring the determinate growth habit in the plants of the present disclosure.

At least 6 SNP markers, i.e. the markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6), were found to be closely linked to the locus conferring the determinate growth habit on chromosome 15 at less than 300 kb in a physical map, as presented in Table 1.

Molecular markers have proven to be of great value for increasing the speed and efficiency of plant breeding. Most traits of agronomic value, e.g. pest resistance, yield and the like, are difficult to measure, often requiring a full growth season and statistical analysis of field trial results. Interpretation of the data can be obscured or confused by environmental variables.

Occasionally it has been possible for breeders to make use of conventional markers such as flower color which could be readily followed through the breeding process. If the desired QTL is linked closely enough to a conventional marker, the likelihood of recombination occurring between them is sufficiently low that the QTL and the marker co-segregate throughout a series of crosses. The marker becomes, in effect, a surrogate for the QTL itself. Prior to the advent of molecular markers, the opportunities for carrying out marker-linked breeding were severely limited by the lack of suitable markers mapping sufficiently close to the desired trait. Genetic map distance is simply a function of recombination frequency between two markers, QTLs or markers and QTLs. Consequently, if a marker and a QTL map too far apart, too much recombination will occur during a series of crosses or self-pollinations such that the marker becomes no longer associated with the QTL. Having a wide selection of molecular markers available throughout the genetic map provides breeders the means to follow almost any desired trait through a series of crosses, by measuring the presence or absence of a marker linked to the QTL which affects that trait. The primary obstacle is the initial step of identifying a linkage between a marker and a QTL affecting the desired trait.

With molecular biology techniques advanced, new markers (i.e. SNP) on a QTL map can be identified by whole genome sequencing (WGS) or specific locus amplified fragment sequencing (SLAF-seq). Inventors conducted quantitative trait locus (QTL) mapping on a population (two BC3F2 families named V15 and V17) to identify QTLs associated with determinate growth habit. Seq-BSA analysis was conducted on the same BC3F2 population to identify the single nucleotide polymorphism (SNP) markers linked to the determinate growth habit. The physical map with positioned SNP markers provides a physical distance between markers on QTL associated with a desired trait.

The inventors of the present disclosure identified such molecular markers that are tightly linked to the determinate growth habit, which brings huge advantage in the breeding program targeting improve the determinate growth habit in cucurbit plants. Molecular markers provide two additional operational advantages. First, since they exist as features of the plant DNA itself, they can be detected soon after germination, for example by analysis of leaf DNA of seedlings. Selection for plants carrying the marker can be performed at the seedling stage, thereby saving the space and energy formerly needed to grow large numbers of plants to maturity. Second, molecular markers do not depend on gene expression for detection. Their use is unlikely to lead to misleading results, such as can occur when environmental or other variables modify expression of conventional marker genes.

More molecular markers can be developed by using the determinate plants of the present disclosure. In general, as the map distance (expressed by the unit cM on a genetic map or by kb on a physical map) between a molecular marker and a gene of interest becomes shorter, the marker and the gene are more closely localized to each other, and more likely to be inherited simultaneously; thus such markers are more useful. Methods of developing molecular markers are well known to one of ordinary skill in the art. Also, specific locus amplified fragment sequencing (SLAF-seq) can be used to identify actual sequence information of SNP markers and physical location on a physical map.

The markers can be bi-allelic dominant, bi-allelic co-dominant, and/or multi-allelic co-dominant. The types of molecular markers that can be developed include, but are not limited to, restriction fragment length polymorphisms (RFLPs), isozyme markers, allele specific hybridization (ASH), amplified variable sequences of plant genome, self-sustained sequence replication, simple sequence repeat (SSR), single base-pair change (single nucleotide polymorphism, SNP), random amplification of polymorphic DNA (RAPDs), SSCPs (single stranded conformation polymorphisms); amplified fragment length polymorphisms (AFLPs) and microsatellites DNA.

Methods of developing molecular markers and their applications are described by Avise (Molecular markers, natural history, and evolution, Publisher: Sinauer Associates, 2004, ISBN0878930418, 9780878930418), Srivastava et al. (Plant biotechnology and molecular markers, Publisher: Springer, 2004, ISBN1402019114, 9781402019111), and Vienne (Molecular markers in plant genetics and biotechnology, Publisher: Science Publishers, 2003), each of which is incorporated by reference in its entirety.

Thus, the present disclosure provides at least one molecular marker that is closely linked to the locus of the determinate plants of the present disclosure. In some embodiments, said molecular marker is chosen from the group consisting of markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6), and any other markers within a chromosomal region delimited by markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6), or a chromosomal region delimited by markers SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

The molecular markers of the present disclosure are closely linked to a desired trait taught herein. As used herein, the phrase “closely linked” or “tightly linked” refers to the situation wherein the genetic distance between the molecular marker and the locus associated with determinate growth habit is less than 5 centimorgan (cM). For example, the genetic distance between the marker and the QTL is about 4.9 cM, 4.8 cM, 4.7 cM, 4.6 cM, 4.5 cM, 4.4 cM, 4.3 cM, 4.2 cM, 4.1 cM, 4.0 cM, about 3.9 cM, 3.8 cM, 3.7 cM, 3.6 cM, 3.5 cM, 3.4 cM, 3.3 cM, 3.2 cM, 3.1 cM, 3.0 cM, about 2.9 cM, 2.8 cM, 2.7 cM, 2.6 cM, 2.5 cM, 2.4 cM, 2.3 cM, 2.2 cM, 2.1 cM, 2.0 cM, about 1.9 cM, about 1.8 cM, about 1.7 cM, about 1.6 cM, about 1.5 cM, about 1.4 cM, about 1.3 cM, about 1.2 cM, about 1.1 cM, about 1.0 cM, about 0.9 cM, about 0.8 cM, about 0.7 cM, about 0.6 cM, about 0.5 cM, about 0.4 cM, about 0.3 cM, about 0.2 cM, about 0.1 cM, or less than 0.1 cM.

The molecular markers of the present disclosure are located on a specific chromosome to the locus associated with the determinate growth habit. For example, the physical distance between the marker and the QTL is less than 500 kb, less than 450 kb, less than 400 kb, less than 350 kb, less than 300 kb, less than 250 kb, less than 200 kb, less than 150 kb, less than 100 kb, less than 50 kb, less than 40 kb, less than 30 kb, less than 20 kb, or less than 10 kb.

The molecular markers identified herein can be used in many aspects of the present disclosure. For example, the molecular markers can be used to assist a breeding program wherein the goal is to transfer determinacy in the cucurbit lines of the present disclosure to other cucurbit lines. Detailed methods of molecular marker assistant selection/breeding is described by Wenzel (Molecular Marker Systems in Plant Breeding and Crop Improvement, Volume 55 of Biotechnology in Agriculture and Forestry, Publisher: Springer, 2007, ISBN 3540740066, 9783540740063), Xu (Molecular Plant Breeding, CABI, February 2010, ISBN 1845933923, 9781845933920), and Kang (Quantitative genetics, genomics, and plant breeding, CABI Publishing Series, 2002, ISBN 0851996019, 9780851996011), each of which is incorporated by reference in its entirety.

### Breeding Methods (Plant Breeding Techniques)

i. Backcross Breeding

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype recurrent parent and the trait of interest from the donor parent are selected and repeatedly crossed (backcrossed) to the recurrent parent. The resulting plant is expected to have the attributes of the recurrent parent (e.g., cultivar) and the desirable trait transferred from the donor parent.

When the Cucurbita plant is used in the context of the present disclosure, this also includes any Cucurbita plant where one or more desired trait has been introduced through backcrossing methods, whether such trait is a naturally occurring one, a spontaneously or artificially induced gene mutation(s), or a gene or a nucleotide sequence modified by the use of New Breeding Techniques. Backcrossing methods can be used with the present disclosure to improve or introduce one or more characteristic into the Cucurbita plant of the present disclosure. The term “backcrossing” as used herein refers to the repeated crossing of a hybrid progeny back to the recurrent parent, i.e., backcrossing one, two, three, four, five, six, seven, eight, nine, or more times to the recurrent parent. The parental Cucurbita plant plant which contributes the gene, the genes, or QTL(s) for the desired characteristic is termed the nonrecurrent or donor parent. This terminology refers to the fact that the nonrecurrent parent is used one time in the backcross protocol and therefore does not recur. The parental Cucurbita plant to which the gene, genes, or QTL(s) from the nonrecurrent parent are transferred is known as the recurrent parent as it is used for several rounds in the backcrossing protocol.

In a typical backcross protocol, the original cultivar of interest (recurrent parent) is crossed to a second cultivar (nonrecurrent parent) that carries the gene or genes of interest to be transferred. The resulting progeny from this cross are then crossed again to the recurrent parent and the process is repeated until a Cucurbita plant is obtained wherein all the desired morphological and physiological characteristics of the recurrent parent are recovered in the converted plant, when grown in the same environmental conditions, in addition to the gene, genes or QTL(s) transferred from the nonrecurrent parent. It has to be noted that one, two, three or more self-pollination and growing of population might be included between two successive backcrosses. Indeed, an appropriate selection in the population produced by the self-pollination, i.e. selection for the desired trait and physiological and morphological characteristics of the recurrent parent might be equivalent to one, two or even three additional backcrosses in a continuous series without rigorous selection, saving then time, money and effort to the breeder. A non-limiting example of such a protocol would be the following: a) the first generation F1 produced by the cross of the recurrent parent A by the donor parent B is backcrossed to parent A, b) selection is practiced for the plants having the desired trait of parent B, c) selected plant are self-pollinated to produce a population of plants where selection is practiced for the plants having the desired trait of parent B and physiological and morphological characteristics of parent A, d) the selected plants are backcrossed one, two, three, four, five, six, seven, eight, nine, or more times to parent A to produce selected backcross progeny plants comprising the desired trait of parent B and the physiological and morphological characteristics of parent A. Step (c) may or may not be repeated and included between the backcrosses of step (d).

The selection of a suitable recurrent parent is an important step for a successful backcrossing procedure. The goal of a backcross protocol is to alter or substitute one or more trait(s) or characteristic(s) in the original inbred parental line in order to find it then in the hybrid made thereof. To accomplish this, a gene, genes or QTL(s) of the recurrent inbred is modified or substituted with the desired gene, genes, or QTL(s) from the nonrecurrent parent, while retaining essentially all of the rest of the desired genetic, and therefore the desired physiological and morphological, constitution of the original inbred. The choice of the particular nonrecurrent parent will depend on the purpose of the backcross; one of the major purposes is to add some commercially desirable, agronomically important trait(s) to the plant. The exact backcrossing protocol will depend on the characteristic(s) or trait(s) being altered to determine an appropriate testing protocol. Although backcrossing methods are simplified when the characteristic being transferred is a single gene and dominant allele, multiple genes and recessive allele(s) may also be transferred and therefore, backcross breeding is by no means restricted to character(s) governed by one or a few genes. In fact the number of genes might be less important that the identification of the character(s) in the segregating population. In this instance it may then be necessary to introduce a test of the progeny to determine if the desired characteristic(s) has been successfully transferred. Such tests encompass visual inspection, simple crossing, but also follow up of the characteristic(s) through genetically associated markers and molecular assisted breeding tools. For example, selection of progeny containing the transferred trait is done by direct selection, visual inspection for a trait associated with a dominant allele, while the selection of progeny for a trait that is transferred via a recessive allele require selfing the progeny to determine which plant carry the recessive allele(s).

Many single gene traits have been identified that are not regularly selected for in the development of a new Cucurbita plant but that can be improved by backcrossing techniques. Single gene traits may or may not be transgenic. Traits for resistance or tolerance to an infection by a virus, a bacterium, an insect or a fungus may also be introduced. Such traits may come from another Cucurbita plant, or a different plant species.

The backcross breeding method provides a precise way of improving varieties that excel in a large number of attributes but are deficient in a few characteristics. (Page 150 of the Pr. R. W. Allard's 1960 book, published by John Wiley & Sons, Inc, Principles of Plant Breeding). The method makes use of a series of backcrosses to the variety to be improved during which the character or the characters in which improvement is sought is maintained by selection. At the end of the backcrossing the gene, genes, or QTL(s) being transferred unlike all other genes, will be heterozygous. Selfing after the last backcross produces homozygosity for this gene, genes, or QTL(s), coupled with selection, will result in a new Cucurbita plant with exactly or essentially the same adaptation, yielding ability and quality characteristics of the recurrent parent but superior to that parent in the particular characteristic(s) for which the improvement program was undertaken. Therefore, this method provides the plant breeder with a high degree of genetic control of this work.

The method is scientifically exact because the morphological and agricultural features of the improved variety could be described in advance and because a similar variety could, if it were desired, be bred a second time by retracing the same steps (Briggs, “Breeding wheats resistant to bunt by the backcross method”, 1930 Jour. Amer. Soc. Agron., 22: 289-244). Backcrossing is a powerful mechanism for achieving homozygosity and any population obtained by backcrossing must rapidly converge on the genotype of the recurrent parent. When backcrossing is made the basis of a plant breeding program, the genotype of the recurrent parent will be theoretically modified only with regards to genes being transferred, which are maintained in the population by selection. One of the advantages of the backcross method is that the breeding program can be carried out in almost every environment that will allow the development of the character being transferred or when using molecular markers that can identify the trait of interest.

ii. Open-Pollinated Populations.

The improvement of open-pollinated populations of such crops as squash, rye, many maize and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed “en masse” by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

iii. Recurrent Selection and Mass Selection

Recurrent selection is a method used in a plant breeding program to improve a population of plants. Cucurbita plants having the semi-determinate or determinate phenotype are suitable for use in a recurrent selection program. The method entails individual plants cross-pollinating with each other to form progeny. The progenies are grown and the superior progenies selected by any number of selection methods, which include individual plant, half-sib progeny, full-sib progeny, and selfed progeny. The selected progenies are cross-pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic variety. A synthetic variety is the resultant progeny formed by the intercrossing of several selected varieties.

Mass selection is a useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk, and then using a sample of the seed harvested in bulk to plant the next generation. Also, instead of self-pollination, directed pollination could be used as part of the breeding program.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Mass and recurrent selections can be used to improve populations of either self- or cross-pollinating plants. A genetically variable population of heterozygous individuals is either identified, or created, by intercrossing several different parents. The best plants are selected based on individual superiority, outstanding progeny, or excellent combining ability. The selected plants are intercrossed to produce a new population in which further cycles of selection are continued.

B) Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

iv. Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the production of both the parental lines and the hybrids which result from crossing those lines. For a detailed discussion of the hybrid production process, see, e.g., Wright, Commercial Hybrid Seed Production 8:161-176, In Hybridization of Crop Plants.

v. Bulk Segregation Analysis (BAS).

BAS, a.k.a. bulked segregation analysis, or bulk segregant analysis, is a method is described by Michelmore et al. (Michelmore et al., 1991, Proceedings of the National Academy of Sciences, USA, 99:9828-9832) and Quarrie et al. (Quarrie et al., 1999, Journal of Experimental Botany, 50(337):1299-1306).

For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., determinate), and the other from the individuals having reversed phenotype (e.g., indeterminate), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

vi. Pedigree Breeding

Pedigree breeding starts with the crossing of two genotypes, such as Cucurbita having semi-determinate or determinate trait and another different Cucurbita having one or more desirable characteristics that is lacking or which complements the semi-determinate or determinate trait. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically in the pedigree method of breeding, five or more successive filial generations of selfing and selection is practiced: F₁ to F₂; F₂ to F₃; F₃ to F₄; F₄ to F₅; etc. After a sufficient amount of inbreeding, successive filial generations will serve to increase seed of the developed variety. Preferably, the developed variety comprises homozygous alleles at about 95% or more of its loci.

vii. Breeding with Molecular Markers

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection. Molecular markers may also be used to identify and exclude certain sources of germplasm as parental varieties or ancestors of a plant by providing a means of tracking genetic profiles through crosses. Molecular markers, which includes markers identified through the use of techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), and Single Nucleotide Polymorphisms (SNPs), may be used in plant breeding methods utilizing Cucurbita plants having the busy-like determinate phenotype. See Vainstein, “Breeding for Ornamentals: Classical and Molecular Approaches,” Kluwer Academic Publishers (2002).

viii. Genetic Marker Profile Through SSR and First Generation Progeny

In addition to phenotypic observations, a plant can also be identified by its genotype. The genotype of a plant can be characterized through a genetic marker profile which can identify plants of the same variety, or a related variety, or be used to determine or validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and Single Nucleotide Polymorphisms (SNPs), all of which are well-known in the art.

ix. Tissue Culture

Further reproduction of the variety can occur by tissue culture and regeneration. Tissue culture of various tissues of ornamental plants and Cucurbita plants and regeneration of plants therefrom is well-known and widely published. For example, reference may be had to do Chandel et al., Advances in Plant Sciences. 2000, 13: 1, 11-17, Costa et al., Plant Cell Report. 2000, 19: 3327-332, Plastira et al., ActaHorticulturae. 1997, 447, 231-234, Zagorska et al., Plant Cell Report. 1998, 17: 12 968-973, Asahura et al., Breeding Science. 1995, 45: 455-459, Chen et al., Breeding Science. 1994, 44: 3, 257-262, Patil et al., Plant and Tissue and Organ Culture. 1994, 36: 2, 255-258. Thus, another embodiment is to provide cells which upon growth and differentiation produce Cucurbita plants having the physiological and morphological characteristics of Cucurbita having semi-determinate or determinate trait described in the present application.

Regeneration refers to the development of a plant from tissue culture. The term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as pollen, ovules, embryos, protoplasts, meristematic cells, callus, leaves, anthers, cotyledons, hypocotyl, pistils, roots, root tips, flowers, seeds, petiole, shoot, or stems, and the like. Means for preparing and maintaining plant tissue culture are well-known in the art.

x. Targeting Induced Local Lesions in Genomes (TILLING)

Breeding schemes of the present application can include crosses with TILLING® plant lines. TILLING® is a method in molecular biology that allows directed identification of mutations in a specific gene. TILLING® was introduced in 2000, using the model plant Arabidopsis thaliana. TILLING® has since been used as a reverse genetics method in other organisms such as zebrafish, corn, wheat, rice, soybean, tomato and lettuce. In some embodiments, TILLING can be applied to Cucurbita plants.

The method combines a standard and efficient technique of mutagenesis with a chemical mutagen (e.g., Ethyl methanesulfonate (EMS)) with a sensitive DNA screening-technique that identifies single base mutations (also called point mutations) in a target gene. EcoTILLING is a method that uses TILLING® techniques to look for natural mutations in individuals, usually for population genetics analysis (see Comai, et al., 2003 The Plant Journal 37, 778-786; Gilchrist et al. 2006 Mol. Ecol. 15, 1367-1378; Mejlhede et al. 2006 Plant Breeding 125, 461-467; Nieto et al. 2007 BMC Plant Biology 7, 34-42, each of which is incorporated by reference hereby for all purposes). DEcoTILLING is a modification of TILLING® and EcoTILLING which uses an inexpensive method to identify fragments (Garvin et al., 2007, DEco-TILLING: An inexpensive method for SNP discovery that reduces ascertainment bias. Molecular Ecology Notes 7, 735-746).

The TILLING® method relies on the formation of heteroduplexes that are formed when multiple alleles (which could be from a heterozygote or a pool of multiple homozygotes and heterozygotes) are amplified in a PCR, heated, and then slowly cooled. As DNA bases are not pairing at the mismatch of the two DNA strands (the induced mutation in TILLING® or the natural mutation or SNP in EcoTILLING), they provoke a shape change in the double strand DNA fragment which is then cleaved by single stranded nucleases. The products are then separated by size on several different platforms.

Several TILLING® centers exists over the world that focus on agriculturally important species: UC Davis (USA), focusing on Rice; Purdue University (USA), focusing on Maize; University of British Columbia (CA), focusing on Brassica napus; John Innes Centre (UK), focusing on Brassica rapa; Fred Hutchinson Cancer Research, focusing on Arabidopsis; Southern Illinois University (USA), focusing on Soybean; John Innes Centre (UK), focusing on Lotus and Medicago; and INRA (France), focusing on Pea and Tomato.

More detailed description on methods and compositions on TILLING® can be found in U.S. Pat. No. 5,994,075, US 2004/0053236 A1, WO 2005/055704, and WO 2005/048692, each of which is hereby incorporated by reference for all purposes.

Thus, in some embodiments, the breeding methods of the present disclosure include breeding with one or more TILLING plant lines with one or more identified mutations.

xi. Mutation Breeding

Mutation breeding is another method of introducing new variation and subsequent traits into cantaloupe plants. Mutations that occur spontaneously or are artificially induced can be useful sources of variability for a plant breeder. The goal of artificial mutagenesis is to increase the rate of mutation for a desired characteristic. Mutation rates can be increased by many different means or mutating agents including temperature, long-term seed storage, tissue culture conditions, radiation (such as x-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in W. R. Fehr, 1993, Principles of Cultivar Development, Macmillan Publishing Co.

xii. New Breeding Techniques

New breeding techniques such as the ones involving the uses of Zinc Finger Nucleases or oligonucleotide directed mutagenesis shall also be used to generate genetic variability and introduce new traits into cantaloupe varieties.

New breeding techniques (NBTs) are said of various new technologies developed and/or used to create new characteristics in plants through genetic variation, the aim being targeted mutagenesis, targeted introduction of new genes or gene silencing. The following breeding techniques are within the scope of NBTs: targeted sequence changes facilitated thru the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM, a.k.a., site-directed mutagenesis), Cisgenesis and intragenesis, epigenetic approaches such as RNA-dependent DNA methylation (RdDM, which does not necessarily change nucleotide sequence but can change the biological activity of the sequence), Grafting (on GM rootstock), Reverse breeding, Agro-infiltration for transient gene expression (agro-infiltration “sensu stricto”, agro-inoculation, floral dip), genome editing with endonucleases such as chemical nucleases, meganucleases, ZFNs, and Transcription Activator-Like Effector Nucleases (TALENs, see U.S. Pat. Nos. 8,586,363 and 9,181,535, incorporated by reference in their entireties), the CRISPR/Cas system (using such as Cas9, Cas12a/Cpf1, Cas13/C2c2, CasX and CasY; see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease, engineered homing endonucleases, DNA guided genome editing (Gao et al., Nature Biotechnology (2016), incorporated by reference in its entirety), and Synthetic genomics. A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques-State-of-the-art and prospects for commercial development”, which is incorporated by reference in its entirety.

xiii. Targeted Gene Editing

Targeted gene editing can be done using CRISPR/Cas9 technology (Saunders & Joung, Nature Biotechnology, 32, 347-355, 2014). CRISPR is a type of genome editing system that stands for Clustered Regularly Interspaced Short Palindromic Repeats. This system and CRISPR-associated (Cas) genes enable organisms, such as select bacteria and archaea, to respond to and eliminate invading genetic material. Ishino, Y., et al. J. Bacteriol. 169, 5429-5433 (1987). These repeats were known as early as the 1980s in E. coli, but Barrangou and colleagues demonstrated that S. thermophilus can acquire resistance against a bacteriophage by integrating a fragment of a genome of an infectious virus into its CRISPR locus. Barrangou, R., et al. Science 315, 1709-1712 (2007). Many plants have already been modified using the CRISPR system. See for example, U.S. Application Publication No. WO2014068346 (György et al., Identification of a Xanthomonas euvesicatoria resistance gene from pepper (Capsicum annuum) and method for generating plants with resistance), Martinelli, F. et al., “Proposal of a Genome Editing System for Genetic Resistance to Tomato Spotted Wilt Virus” American Journal of Applied Sciences 2014, and Noman, A. et al., “CRISPR-Cas9: Tool for Qualitative and Quantitative Plant Genome Editing” Frontiers in Plant Science Vol. 7 Nov. 2016.

Gene editing can also be done using crRNA-guided surveillance systems for gene editing. Additional information about crRNA-guided surveillance complex systems for gene editing can be found in the following documents, which are incorporated by reference in their entirety: U.S. Application Publication No. 2010/0076057 (Sontheimer et al., Target DNA Interference with crRNA); U.S. Application Publication No. 2014/0179006 (Feng, CRISPR-CAS Component Systems, Methods, and Compositions for Sequence Manipulation); U.S. Application Publication No. 2014/0294773 (Brouns et al., Modified Cascade Ribonucleoproteins and Uses Thereof); Sorek et al., Annu. Rev. Biochem. 82:273-266, 2013; and Wang, S. et al., Plant Cell Rep (2015) 34: 1473-1476. Therefore, it is another embodiment to use the CRISPR system on the plants of the instant application to for example, modify traits and tolerances to pests and viruses.

The present disclosure teaches a method comprising mutagenesis of the plant, or plant part thereof. In some embodiments, said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.

Accordingly, the present disclosure is also directed to the use of a semi-determinate or determinate Cucurbita plant as defined above, as a breeding partner in a breeding program for obtaining semi-determinate or determinate Cucurbita plants from indeterminate Cucurbita plants. Indeed, such a semi-determinate or determinate Cucurbita plant harbors in its genome one QTL on chromosome 15 as defined here above conferring said determinacy. In an embodiment, such a determinate Cucurbita plant harbors in its genome at least one or more of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6).

In another embodiment, such a determinate Cucurbita plant harbors in its genome all of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) at homozygous state. In still another embodiment, such a determinate Cucurbita plant harbors in its genome a combination of at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). Instill another embodiment, such a determinate Cucurbita plant harbors in its genome any combination of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6).

By crossing this plant with indeterminate or less determinate plants, it is possible to transfer this QTL. A plant according to the disclosure can thus be used as a breeding partner for introgressing the QTL on chromosome 15 conferring the desired phenotype. In some embodiments, a plant according to the disclosure can be used as a breeding partner for introgressing the QTL on chromosome 15 as defined above conferring the desired phenotype. In some embodiments, a plant according to the disclosure can be used as a breeding partner for introgressing at least one or more of the following alleles on chromosome 15: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6). In some embodiments, a plant according to the disclosure can be used as a breeding partner for introgressing allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) on chromosome 15.

The disclosure is also directed to the same use with plants or seeds of Cucurbita plant as deposited at NCIMB accession no. 43740. Said plants are also suitable as introgression partners in a breeding program aiming at conferring the desired phenotype to a Cucurbita plant or germplasm.

In such a breeding program, the selection of the progeny displaying the desired phenotype, or bearing QTL linked to the desired phenotype, can advantageously be carried out on the basis of the allele of the marker disclosed here above. The progeny is selected on the presence of one or more of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) on chromosome 15. In some embodiments, the progeny is selected on the presence of all of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) on chromosome 15. In some embodiments, the progeny is selected on the presence of at least at least two of the following alleles: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) on chromosome 15 on chromosome 15. In another embodiment, the progeny is selected on the presence of any combination of the following alleles at homozygous state: allele C of marker SQ-0018902-N (SEQ ID NO:7), allele A of marker SQ-0018903-N (SEQ ID NO:8), allele C of marker SQ-0018904-N (SEQ ID NO:9), allele C of marker SQ-0018907-N (SEQ ID NO:10), allele T of marker SQ-0018909-N (SEQ ID NO:11), allele T of marker SQ-0018914-N (SEQ ID NO:12), and allele A of marker SQ-0018839 (SEQ ID NO:6) on chromosome 15.

A plant according to the disclosure, or as deposited under NCIMB accession no. 43740, is thus particularly valuable in a marker-assisted selection program for obtaining commercial Cucurbita lines and determinate varieties. The disclosure is also directed to the use of said plants in a program aiming at identifying, sequencing and/or cloning the genes conferring the desired phenotype, i.e. determinate growth habit.

The present disclosure further provides methods producing a genus Cucurbita plant with a determinate growth habit, the method comprising: (i) crossing a first genus Cucurbita plant with a second genus Cucurbita plant to produce progeny plants of a subsequent generation, (ii) selecting one or more progeny plants that contain a determinate growth habit from the progeny of the subsequent generation, wherein the first genus Cucurbita plant comprises said determinate growth habit, (iii) backcrossing the selected progeny plants having said determinate growth habit or selfed offspring thereof with the second Cucurbita plant or a third Cucurbita plant to produce backcross progeny plants, (iv) selecting for backcross progeny plants having said determinate growth habit from the backcross progeny plants, and (v) repeating steps (iii) and (iv) at least one time in succession to produce selected second or higher backcross progeny plants that confer said determinate growth habit, wherein the progeny plants selected in (ii) and (iv) are selected by assaying for the presence of one or more of the markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said first genus Cucurbita plant is a plant conferring a determinate growth habit. In other embodiments, said second genus Cucurbita plant is a plant conferring an indeterminate growth habit.

In some embodiments, the first genus Cucurbita plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the first genus Cucurbita plant is a Cucurbita moschata (C. moschata), Cucurbita pepo (C. pepo) or a Cucurbita maxima (C. maxima) plant.

In other embodiments, the second genus Cucurbita plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the second genus Cucurbita plant is a Cucurbita moschata (C. moschata), Cucurbita pepo (C. pepo) or a Cucurbita maxima (C. maxima) plant.

In further embodiments, the third genus Cucurbita plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the third genus Cucurbita plant is a Cucurbita moschata (C. moschata), Cucurbita pepo (C. pepo) or a Cucurbita maxima (C. maxima) plant.

In some embodiments, said C. moschata plant is butternut squash (e.g. C. moschata butternut type, C. moschata long neck butternut type), C. moschata flat round type, C. moschata round to oblate type, or hybrid thereof.

In other embodiments, the second or third genus Cucurbita plant is a commercial elite plant that has an indeterminate growth habit. In further embodiments, the second or third genus Cucurbita plant is a Cucurbita plant, for example, TARMINO, VICTORIA, GLORIA, CORA, SINATRA, MUSA, LOREA, ASMA, JEDIDA, FALALI, SIBELLE, GLADIATOR, MAGIC LANTERN, ALADDIN, KRATOS, WARTY GOBLIN, MISCHIEF, WALTHAM, ZYPMB24, HOWDEN BIGGIE, F1 ROYAL ACE PM, F1 FLORENCIA, F1 MILANO, AKAI BOTCHAN, KURIJIMAN, or hybrid thereof.

Provided is a selected genus Cucurbita plant produced by the method taught herein, wherein said plant has a determinate growth habit.

Also, provided is a plant, plant part, or plant cell derived from the genus Cucurbita plant produced by the method taught herein. A seed is also produced by the genus Cucurbita plant produced by the method taught herein.

The present disclosure provides a method for identifying a Cucurbita plant comprising a QTL associated with a determinate growth habit, the method comprising: (i) providing a population of cultivated Cucurbita plants, (ii) screening said population using a molecular marker assay which detects at least one SNP marker selected from the group consisting of: SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and, SQ-0018839 (SEQ ID NO:6) or any combinations thereof and (iii) identifying and/or selecting a plant comprising at least one of the SNP markers of step (ii).

Accordingly, the present disclosure provides plants of the genus Cucurbita produced by the breeding methods described above. In some further embodiments, the present disclosure provides a plant, plant part, or plant cell of the genus Cucurbita derived from the plant of the genus Cucurbita produced by the breeding methods described above, for example, plant seeds or fruits of the genus Cucurbita derived from said selected plant of the genus Cucurbita.

The present disclosure also provides a method for producing a squash plant with a determinate growth habit, the method comprising: (i) crossing a first squash plant with a second squash plant to produce progeny plants of a subsequent generation, (ii) selecting one or more progeny plants that contain a determinate growth habit from the progeny of the subsequent generation, wherein the first squash plant comprises said determinate growth habit, (iii) backcrossing the selected progeny plants having said determinate growth habit or selfed offspring thereof with the second squash plant or a third squash plant to produce backcross progeny plants, (iv) selecting for backcross progeny plants having said determinate growth habit from the backcross progeny plants, and (v) repeating steps (iii) and (iv) at least one time in succession to produce selected second or higher backcross progeny plants that confer said determinate growth habit. In some embodiments, the progeny plants selected in (ii) and (iv) are selected by assaying for the presence of one or more of the markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO:12), and SQ-0018839 (SEQ ID NO:6).

In some embodiments, said first squash plant is a plant conferring a determinate growth habit. In other embodiments, said second squash plant is a plant conferring an indeterminate growth habit.

In some embodiments, the first squash plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the first squash plant is a Cucurbita moschata (C. moschata), Cucurbita pepo (C. pepo) or a Cucurbita maxima (C. maxima) plant.

In other embodiments, the second squash plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the second squash plant is a Cucurbita moschata (C. moschata), Cucurbita pepo (C. pepo) or a Cucurbita maxima (C. maxima) plant.

In further embodiments, the third squash plant is a Cucurbita moschata (C. moschata), Cucurbita pepo (C. pepo), Cucurbita okeechobeensis (C. okeechobeensis), Cucurbita pedatifolia (C. pedatifolia) or a Cucurbita maxima (C. maxima) plant. In further embodiments, the third squash plant is a Cucurbita moschata (C. moschata), Cucurbita pepo (C. pepo) or a Cucurbita maxima (C. maxima) plant.

In some embodiments, said Cucurbita moschata (C. moschata) plant is butternut squash (e.g. C. moschata butternut type, C. moschata long neck butternut type), C. moschata flat round type, C. moschata round to oblate type, or hybrid thereof.

In other embodiments, the second or third squash plant is a commercial elite plant that has an indeterminate growth habit. In further embodiments, the second or third squash is a cucurbita plant, for example, TARMINO, VICTORIA, GLORIA, CORA, SINATRA, MUSA, LOREA, ASMA, JEDIDA, FALALI, SIBELLE, GLADIATOR, MAGIC LANTERN, ALADDIN, KRATOS, WARTY GOBLIN, MISCHIEF, WALTHAM, ZYPMB24, HOWDEN BIGGIE, F1 ROYAL ACE PM, F1 FLORENCIA, F1 MILANO, AKAI BOTCHAN, KURIJIMAN, or hybrid thereof.

Provided is a selected squash plant produced by the method taught herein, wherein said plant has a determinate growth habit.

Also, provided is a plant, plant part, or plant cell derived from the squash plant produced by the method taught herein. A seed is also produced by the squash plant produced by the method taught herein.

Accordingly, the present disclosure provides squash plants produced by the breeding methods described above. In some further embodiments, the present disclosure provides a squash plant, plant part, or plant cell derived from the plant produced by the breeding methods described above, for example, squash plant seeds or fruits derived from said selected plant.

The disclosure also provides the use of the plant according to the disclosure as a breeding partner in a breeding program for obtaining Cucurbita plant conferring a determinate growth habit.

The present disclosure more particularly relates to the breeding of a new typology of squash or pumpkin, i.e. a determinate squash or pumpkin, which allows an increased productivity per acre and the possibility to perform mechanical and concentrated harvest.

The present disclosure teaches that the determinate Cucurbita plant taught herein gives rise to increased productivity per acre. With density increased, growers should produce more tons per acre. Consequently, more seeds per acre can be provided by suppliers using the determinate Cucurbita plant taught herein, thereby benefiting consumers.

Also, the present disclosure teaches that the determinate Cucurbita plant taught herein would make mechanical harvest possible. Today, in practice, butternut is 100% hand harvested, but mechanical harvest is possible only if fruit set in concentrated like in tomato determinate.

The present disclosure provides this innovation with determinacy trait added to the Cucurbita plants could be a breakthrough to develop mechanical and concentrated harvest for processing or even for fresh market (mature fruit with thick skin, not too sensitive to machine bruising).

It should be understood that the examples and embodiments described herein are for illustrative purposes only and that various modifications or changes in light thereof will be suggested to persons skilled in the art and are to be included within the spirit and purview of this application.

### DEPOSIT INFORMATION

A representative sample of seeds from the C. moschata plant according to the disclosure (PUV008) has been deposited by H M. Clause, S. A., Rue Louis Saillant, Z. I. La Motte, BP83, 26802 Portes-lès-Valence cedex, France, under the authorization of the owner of the present disclosure (i.e., Vilmorin & Cie), pursuant to, and in satisfaction of, the requirements of the Budapest Treaty on the International Recognition of the Deposit of Microorganisms for the Purposes of Patent Procedure (the “Budapest Treaty”) with the National Collection of Industrial, Food and Marine Bacteria (NCIMB), Ferguson Building, Craibstone Estate, Busksburn, Aberdeen, Scotland, AB21 9YA, United Kingdom, under accession number NCIMB 43740.

A deposit of the C. moschata seeds is maintained by H M. Clause, S. A., Rue Louis Saillant, Z. I. La Motte, BP83, 26802 Portes-lès-Valence cedex, France, under the authorization of the owner of the present disclosure (i.e., Vilmorin & Cie).

To satisfy the enablement requirements of 35 U.S.C. 112, and to certify that the deposit of the seeds of the present disclosure meets the criteria set forth in 37 CFR 1.801-1.809, Applicants hereby make the following statements regarding the deposit of the resistant germplasm:
1. During the pendency of this application, access to the disclosure will be afforded to the Commissioner upon request;
2. Upon granting of the patent the strain will be available to the public under conditions specified in 37 CFR 1.808;
3. The deposit will be maintained in a public repository for a period of 30 years or 5 years after the last request or for the enforceable life of the patent, whichever is longer; and
4. The viability of the biological material at the time of deposit will be tested; and
5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to the deposit.

### EXAMPLES

### Example 1. Creation of Population of Cucurbita moschata Having Determinate Growth Habit

Inventors observed and characterized the determinate growth habit plant architecture in Cucurbita moschata breeding program inventors conducted.

As presented in FIG. 1, a donor line (A) having determinate growth habit and non-mosaic, round fruit shape was crossed with a first butternut Cucurbita moschata line (B) having indeterminate growth habit and non-mosaic, butternut fruit shape to produce F1 hybrid progenies. The donor line (A) carries the determinate trait, which can be transferred or introgressed into a recipient line (B). The F1 plants were followed by two recurrent backcrosses with a second Cucurbita moschata line (C) having indeterminate growth habit and mosaic, butternut fruit shape. The determinate trait is further transferred or introgress into a second recipient line (C). Then, the BC2F1 progenies were further backcrossed and selfed to generate BC3F2 plant materials. BC3F2 plants were self-pollinated four more times to generate BC3F6 population (a.k.a. PUV008) at a homozygous state for the determinate QTL taught herein in the recurrent line. At Year 1 the necessary germplasms collected and at Year 2 the first observation trial was done. At year 8, the further observation was accomplished and the line was selected and fixed for QTL mapping. Until Year 16, all the necessary crosses for trait(s) integration were carried out including backcross and test crosses to have desired fruit shape and adaptation along with new trait(s) of determinacy. At year 17 this BC3F6 population (PUV008) was QTL mapped and identified for the QTL with the associated markers and/or mutation(s). Seeds of PUV008 cultivar with determinate growth habit have been deposited as NCIMB No. 43740.

PUV008 plant has the following morphologic and physiological characteristics, as described in Table 2, based on data collected during a single growth season in France. All experiments and phenotype collection were done under the direct supervision of the applicant.

TABLE 2

| Trait | Scale | PUV008 |
|---|---|---|
| Growht Habit | Indeterminate, semi- | determinate |
| | determinate, determinate | |
| Plant: Length of main stem | very short, short, medium, | very short |
| | long, very long | |
| Leaf: Silvering | No slivering, lightly silvered, | No silvering |
| | moderately silvered, strongly | |
| | silvered | |
| Leaf: Leaf blade: margin | entire or very weakly incised, | very weakly incised |
| | weakly incised, moderately | |
| | incised | |
| Fruit Length (mm) | | 80 mm |
| Fruit Neck Width (mm) | | N/A |
| Fruit Bulb Width (mm) | | 190 mm |
| Fruit: Peduncle: length | short, medium, long | long |
| Fruit: shape | heart shaped, capped, | retangular |
| | transverse medium elliptic, | |
| | transverse broad elliptic, | |
| | circular, narrow elliptic, | |
| | medium elliptic, rectangular, | |
| | trapezoid, broad pear shaped, | |
| | narrow pear shaped, butternut | |
| | shape, Hubbard type, tri-lobed | |
| Fruit: position of broadest part | towards stem end, at middle, | at middle |
| | towards blossom end | |
| Fruit: profile at stem end | raised, flat, slightly depressed, | moderately depressed |
| | moderately depressed, strongly | |
| | depressed | |
| Fruit: profile at blossom end | depressed, flat, raised | depressed |
| (flower scar included) | | |
| Fruit: grooves | absent, present | present |
| Fruit: distance between | small, medium, large | medium |
| grooves | | |
| Fruit: depth of grooves | shallow, medium, deep | medium |
| Fruit: number of colors of skin | one, two color intensities (with | one |
| | clear borders), two color hues, | |
| | more than two color hues or | |
| | intensities (with clear borders) | |
| Fruit: main color of skin | white, cream, yellow, orange, | tan |
| | red, pink, green, grey green, | |
| | grey, tan | |
| Excluding varieties with main | light, medium, dark | light |
| color of skin: cream or white: | | |
| Fruit: intensity of main color | | |
| of skin | | |
| Only varieties with two or | white, cream, yellow, orange, | NA |
| more color hues: Fruit: | red, pink, green, grey green, | |
| secondary color of skin | grey, tan | |
| Only varieties with two or | light, medium, dark | NA |
| more color hues: Fruit: | | |
| intensity of secondary color of | | |
| skin | | |
| Only varieties with two or | only patches, patches and | NA |
| more hues or intensities: Fruit: | stripes, only stripes | |
| distribution of secondary color | | |
| of skin | | |
| Fruit: texture of surface | smooth, rough, warted, bullate | smooth |

### Example 2. QTL Mapping for Determinate Growth Habit Trait

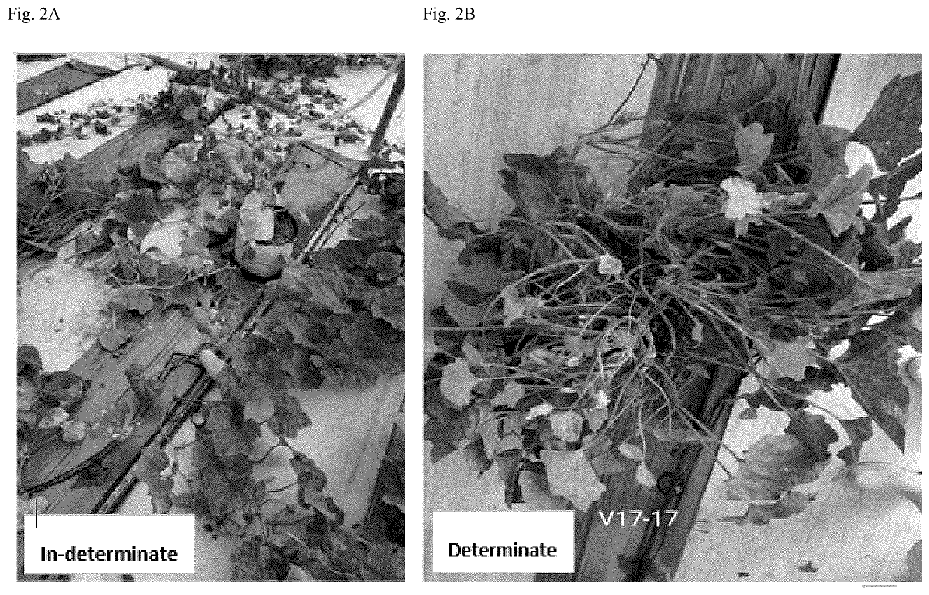

The developed BC3F2 plant material (FIG. 2B) was used to evaluate the trait heritability by phenotyping a number of plants of two BC3F2 families followed by sequencing approach to develop markers linked to determinate growth habit trait. The two BC3F2 families are named as ‘V15’ and ‘V17’ QTL mapping populations.

149 individual plants of BC3F2 segregating for the determinate trait (population V17) was phenotyped. The population was used later for constructing the seq-BSA bulks. At the same time another 149 individual plants of BC3F2 (population V15) was phenotyped in order to check the segregation, but also later on check the accuracy of marker prediction. Both indeterminate recurrent parent and donor parent were included as controls in every trial.

A last trial was scored for several traits to characterize the determinate trait. In this trial, the advanced material (i.e. BC3F6) and recurrent line were evaluated. 30 plants of the in-determinate recurrent genotype and 34 plants of BC3F6 with a determinate architecture were evaluated for (i) The number of internodes; (ii) The length of internode by plant; and (iii) The average length of the internodes. Quantitative trait locus (QTL) mapping on a population (two BC3F2 families named V15 and V17) were conducted to identify QTLs associated with determinate growth habit.

To develop markers, seq-BSA (Sequencing Bulk Segregant Analysis) was chosen. DNA of 20 determinate plants were bulked and 20 DNA of 20 in-determinate plants were bulked. Both bulks and the three parents have been sequenced, then mapped against internal Cucurbita moschata reference genome, followed by SNP calling.

Statistical association between SNP and phenotype of the bulks was run using statistical software.

For bulks construction and sequencing, genomic DNA was isolated from the BC3F2 as well as parents of the population individually using a genomic DNA purification kit (Macherey Nagel). DNA was quantified using PicoGreen measurement. Two bulks were generated by pooling equal amounts of DNA from 20 samples of the population with Determinate trait and 20 with non-determinate. The two bulks of DNA as well the parents were sequenced using the Illumina's NovaSeq sequencing platform.

For variant-calling analysis, raw reads were processed using Trimmomatic to remove low-quality sequencing reads and any adapter contamination. The clean reads were further rechecked for quality using FASTQC (bioinformatics.babraham.ac.uk/projects/fastqc). The high-quality reads were mapped onto internal Cucurbita moschata genome using Burrows-Wheeler Aligner (BWA) MEM tool. Picard Tools ‘SortSam’ was used to sort mapped reads. ‘MarkDuplicates’ was used to locate duplicate molecules and ‘BuildBamIndex’ to index the BAM files with the default parameters SNPs and InDels were called using GATK (v.3.7) ‘HaplotypeCaller’ (DePristo et al., 2011; Van der Auwera et al., 2013) across parental lines and bulks. Variant-calling files from parental lines and bulks were merged using bcftools ‘merge’. Polymorphic SNPs between parental lines were selected using SNPSift and used for SNP-index analysis. After filtering SNP polymorphic between the two parents, 412,183 SNP and 338,478 INDEL were found in-between the two bulks.

The Seq-BSA analysis was performed under R software (R Core Team, 2017) using QTLseqr package (Mansfeld and Grumet, 2018). The Delta SNP-index was used as the key indicator for the QTL discovery. The Delta SNP-index is defined for each SNP as the difference of the “determinate bulk” SNP-index from the “in-determinate” bulk SNP-index. SNP-index of both bulks is defined as the total reference allele frequency. Filters on SNP quality allowed to reduce the number of SNP and INDEL to 10,044. Variants with a total depth below 10 or above 175, and an allele frequency to the reference genome below 0.1 were discarded.

For genotyping, SNP identified through seq-BSA analysis were design to genotype V15 and V17 population and check the profiles of the observed phenotype. An Anova was used to find the most associated SNP with the trait.

Using Seq-BSA analysis, single nucleotide polymorphism (SNP) markers linked to the determinate growth habit on the QTL map were identified on the same BC3F2 population. The analysis of deltaSNP index revealed a peak on chromosome 15. Several SNP have absolute deltaSNP index higher than 0.75 suggesting a QTL. Indeed, high absolute value of the deltaSNP index suggest a major difference of allele frequency between bulks and therefore potential QTL.

143 SNP were designed. The populations of V15 and V17 were genotyped. The results of the Anova revealed six SNP with low p-values suggesting a strong association between the SNP and the phenotype, which are SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5).

From the Seq-BSA analysis, one major QTL on chromosome 15 was identified on two populations BC3F2 (V15 and V17) with a first interval located between markers SQ-0018902-N (SEQ ID NO:7) and SQ-0018839 (SEQ ID NO:6), (i.e. determinate growth habit).

Table 3 presents the coordinate information of SNP markers on chromosome 15 in relation to QTL associated with a desired trait. The positional coordinate information of SNPs in Table 3 is based on a publicly available Cucurbita moschata (Rifu) Genome database (cucurbitgenomics.org/organism/9). deltaSNP index indicates markers having the closest distance to the QTL of interest are selected from SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018902 (SEQ ID NO:1), SQ-0018903 (SEQ ID NO:2), SQ-0018904 (SEQ ID NO:3), SQ-0018907 (SEQ ID NO:4), SQ-0018839 (SEQ ID NO:6), and SQ-0018909 (SEQ ID NO:5), which are associated with the lowest P-values in the populations from the experiment.

TABLE 3
Physical Map Position of QTL markers with deltaSNP index for determinate growth habit trait

| Population | Locus | Chromosome | Position | R2 | P value | deltaSNP | MAF | HetRate |
|---|---|---|---|---|---|---|---|---|
| V15 | SQ-0018902 | 15 | 8473188 | 92% | 7.6E−88 | 0.81 | 0.44 | 50.32 |
| | (SEQ ID | | | | | | | |
| | NO: 1) | | | | | | | |
| V17 | SQ-0018902 | 15 | 8473188 | 88% | 1.1E−74 | 0.81 | 0.49 | 52.53 |
| | (SEQ ID | | | | | | | |
| | NO: 1) | | | | | | | |
| V15 | SQ-0018903 | 15 | 8476073 | 92% | 9.9E−89 | 0.93 | 0.43 | 50.00 |
| | (SEQ ID | | | | | | | |
| | NO: 2) | | | | | | | |
| V17 | SQ-0018903 | 15 | 8476073 | 82% | 2.5E−60 | 0.93 | 0.49 | 53.16 |
| | (SEQ ID | | | | | | | |
| | NO: 2) | | | | | | | |
| V15 | SQ-0018904 | 15 | 8476149 | 92% | 9.9E−89 | 0.94 | 0.43 | 50.00 |
| | (SEQ ID | | | | | | | |
| | NO: 3) | | | | | | | |
| V17 | SQ-0018904 | 15 | 8476149 | 88% | 9.3E−74 | 0.94 | 0.49 | 52.87 |
| | (SEQ ID | | | | | | | |
| | NO: 3) | | | | | | | |
| V15 | SQ-0018907 | 15 | 8477012 | 92% | 2.8E−87 | 0.86 | 0.44 | 50.00 |
| | (SEQ ID | | | | | | | |
| | NO: 4) | | | | | | | |
| V17 | SQ-0018907 | 15 | 8477012 | 89% | 3.6E−76 | 0.86 | 0.48 | 52.90 |
| | (SEQ ID | | | | | | | |
| | NO: 4) | | | | | | | |
| V15 | SQ-0018909 | 15 | 8647279 | 85% | 1.3E−64 | 0.86 | 0.43 | 48.72 |
| | (SEQ ID | | | | | | | |
| | NO: 5) | | | | | | | |

Table 3 shows the raw analysis outputs from the Anova between markers and phenotypes. Each raw gives statistics of the most important markers. The P-value reflects the association between the trait and the marker. At least the 5 SNPs presented in Table 3 are strongly linked to the trait. R² in Table 3 indicates the prediction accuracy of the SNP. Physical position of the SNP is also provided in Table 3. The 5 SNPs of interest are distant of about 300 kb on chromosome 15.

The segregation for the two BC3F2 families named V15 and V17 is presented is Table 4.

TABLE 4
Segregation ratio of traits from two QTL mapping populations

| | V17 (%) | V15 (%) |
|---|---|---|
| In-Determinate | 23.3 | 30.2 |
| Semi-Determinate | 57.3 | 54.4 |
| Determinate | 19.3 | 15.4 |

The segregation presented in Table 4 indicates a “codominant” or “incompletely dominant” determinism type of the trait considering the overall architecture of the determinate plant. This suggests that the genetic mechanism is relatively simple, with one major QTL (minor QTLs can have an effect).

### Example 3. Phenotypic Data of Population of Cucurbita moschata Having Determinate Growth Habit

The recurrent inbred line (35 indeterminate plants) and a BC3F6 population (34 determinate plants; plants carrying the determinate QTL at a homozygous state) were phenotyped to look at plant architecture differences between determinate and in-determinate type.

It was observed significant differences between the length of each internodes, between the average length of the internodes and between the number of internodes produces by the plant.

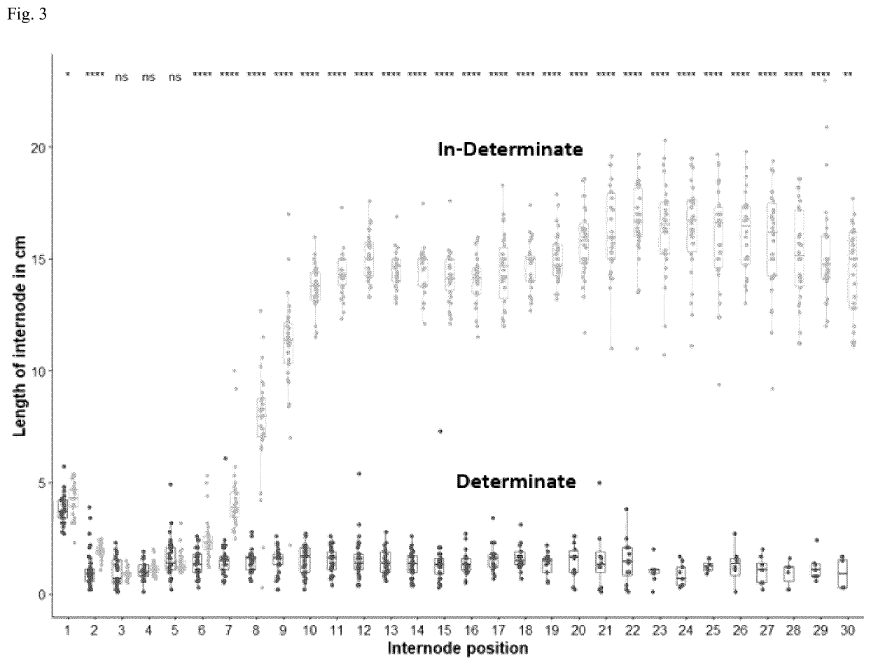

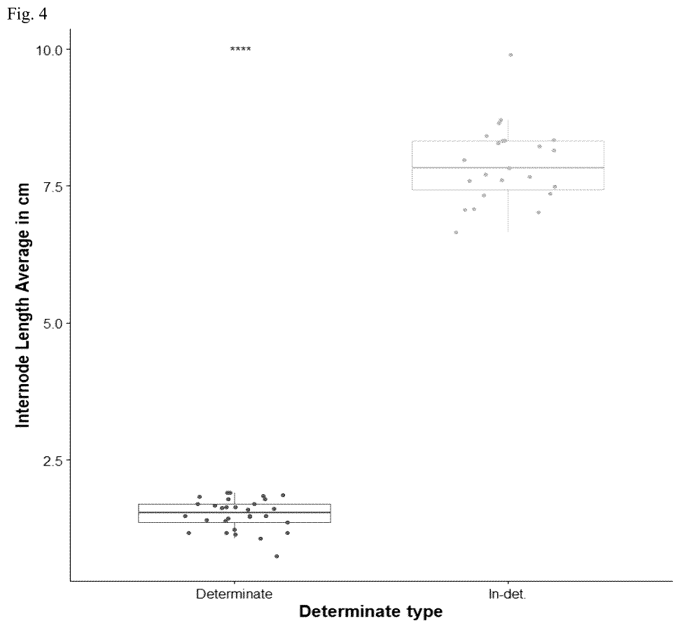

FIG. 4 shows major differences in internode length, between both plant types; in-determinate (upper part in FIG. 3) vs determinate (lower part in FIG. 3), indicating that determinate plants have significantly shorter internode length than in-determinate plants. FIG. 3 displays only the 30 first internodes, indeed since determinate plant stopped growing, there were not enough data scored for the determinate type after the 30^{th} internode. The X-axis represents the internode position on the plant, the Y-axis represent the length of the internode in cm. Each point is the length of one internode of one plant. On the top of the figure, the stars represent the significance level between the two groups.

A wilcoxon test was run between the two groups for each internode presented in FIG. 3. Except for the 3^{rd} to the 5^{th} internode of the plant, the difference in length of internodes is highly significant. The significance level is displayed in Table 5.

TABLE 5
P-value associated to the Wilcoxon test between indeterminate
and determinate plant for each internode length from the 1^{st} to
the 30^{th} internode of each plant population

| Internode | | | |
|---|---|---|---|
| Position | p-value | P significance | Method |
| 1 | 1.67E⁻⁰² | * | Wilcoxon |
| 2 | 2.11E⁻⁰⁸ | **** | Wilcoxon |
| 3 | 6.50E⁻⁰¹ | ns | Wilcoxon |
| 4 | 2.55E⁻⁰¹ | ns | Wilcoxon |
| 5 | 3.90E⁻⁰¹ | ns | Wilcoxon |
| 6 | 8.68E⁻⁰⁷ | **** | Wilcoxon |
| 7 | 6.98E⁻¹⁰ | **** | Wilcoxon |
| 8 | 9.23E⁻¹⁰ | **** | Wilcoxon |
| 9 | 5.54E⁻¹¹ | **** | Wilcoxon |
| 10 | 4.36E⁻¹¹ | **** | Wilcoxon |
| 11 | 4.00E⁻¹¹ | **** | Wilcoxon |
| 12 | 4.32E⁻¹¹ | **** | Wilcoxon |
| 13 | 4.03E⁻¹¹ | **** | Wilcoxon |
| 14 | 6.02E⁻¹¹ | **** | Wilcoxon |
| 15 | 9.70E⁻¹¹ | **** | Wilcoxon |
| 16 | 9.78E⁻¹¹ | **** | Wilcoxon |
| 17 | 1.70E⁻⁰⁹ | **** | Wilcoxon |
| 18 | 1.59E⁻⁰⁸ | **** | Wilcoxon |
| 19 | 3.24E⁻⁰⁸ | **** | Wilcoxon |
| 20 | 1.27E⁻⁰⁷ | **** | Wilcoxon |
| 21 | 5.70E⁻⁰⁷ | **** | Wilcoxon |
| 22 | 5.71E⁻⁰⁷ | **** | Wilcoxon |
| 23 | 1.26E⁻⁰⁶ | **** | Wilcoxon |
| 24 | 7.24E⁻⁰⁶ | **** | Wilcoxon |
| 25 | 1.83E⁻⁰⁵ | **** | Wilcoxon |
| 26 | 1.85E⁻⁰⁵ | **** | Wilcoxon |
| 27 | 1.86E⁻⁰⁵ | **** | Wilcoxon |
| 28 | 4.99E⁻⁰⁵ | **** | Wilcoxon |
| 29 | 4.97E⁻⁰⁵ | **** | Wilcoxon |
| 30 | 1.56E⁻⁰³ | ** | Wilcoxon |

Considering all internodes independently from their positions, FIG. 4 shows difference of internode length average between two plant types; determinate (on the left) and in-determinate (on the right), indicating that determinate plants have significantly shorter internode length than in-determinate plants. Table 6 presents that the difference of internode length average between determinate and in-determinate plants is highly significant, with a P-value of 6.30 E⁻¹⁰. In average, internodes of determinate type plants are shorter than 2.5 cm, whereas those of indeterminate type plants are longer than 6 cm.

TABLE 6
P-value associated to the Wilcoxon test between indeterminate
and determinate plant for internode length average from the 1^{st} to
the 30^{th} internode of each plant population

| Trait | p-value | P significance | Method |
|---|---|---|---|
| Internode Length Average | 6.30E⁻¹⁰ | **** | Wilcoxon |

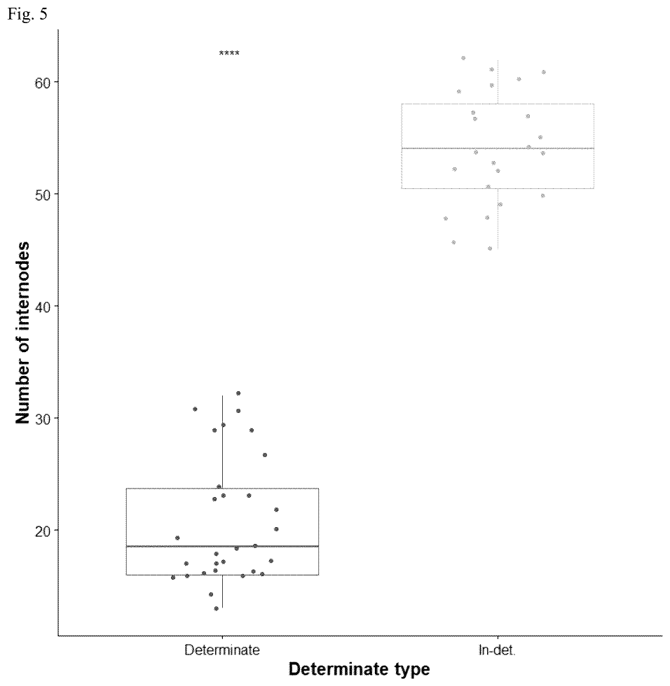

After 2.5 month of growth, the number of internodes for each plant population was scored. Whereas the number of internodes for indeterminate type plants was in average 54.1, the number of internodes for the determinate types plants was in average 20.8. The internode number of the determinate plants is more than a half of that of the in-determinate plants. The difference in internode number is highly significant as presented in FIG. 5 and Table 7.

TABLE 7
P-value associated to the Wilcoxon test between indeterminate
and determinate plant for internode number average from the 1^{st} to
the 30^{th} internode of each plant population

| Trait | p-value | P significance | Method |
|---|---|---|---|
| Number of internode | 5.88E⁻¹⁰ | **** | Wilcoxon |

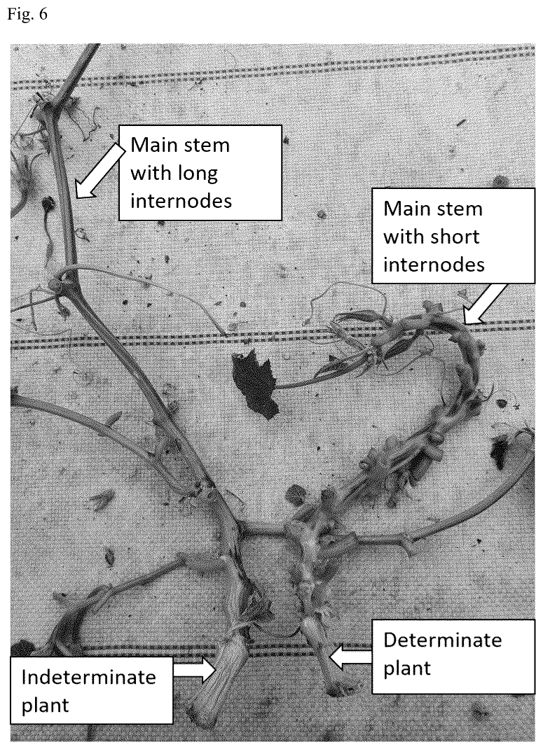

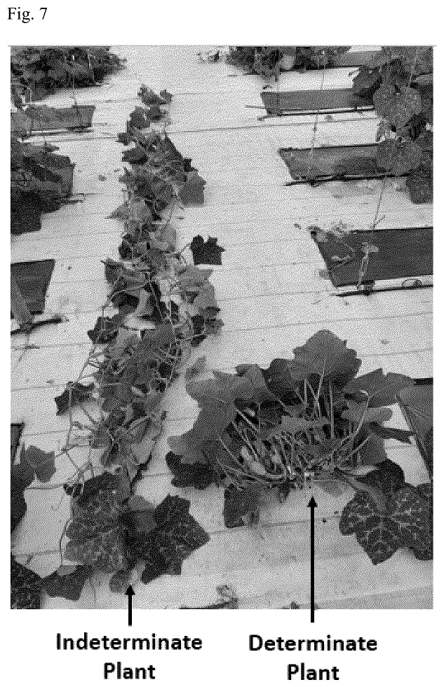

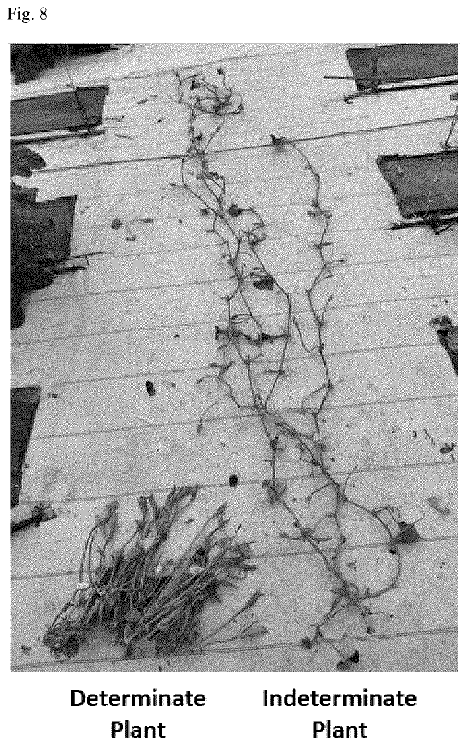

FIGS. 6-8 show one of bushy-like determinate plants from BC3F6 population with a limited number of short internodes in comparison to a vine-like recurrent indeterminate plant that has a large number of long internodes. Table 8 presents numbers of internodes and total length of internodes from 35 recurrent indeterminate plants and 34 BC3F6 determinate plants.

TABLE 8
Comparison of Numbers of internodes
and total length of internodes

| | | | | Total |
|---|---|---|---|---|
| | | | | length of |
| No. of | | | No. of | Internodes |
| plants | Genotype | Phenotype | Internodes | (cm) |
| 35 plants | Inbred | Indeterminate | 48-62 | 18.7 cm-53.3 cm |
| | recurrent | | | |
| | line | | | |
| 34 plants | BC3F6 | Determinate | 16-32 | 318.8 cm-508 cm |

Using six SNP markers described above, BC3F6 population (i.e. determinate plants) and recurrent line (i.e. in-determinate plants) were genotyped. The haplotypes of 30 plants of the in-determinate recurrent genotype are presented in Table 9, which are distinguished from the haplotypes of 34 plants BC3F6 that are determinate. Table 9 shows the haplotypes of both determinate and indeterminate type.

TABLE 9
Haplotypes of both indeterminate and determinate plants on six SNP markers

| No. of | | | SQ- | SQ- | SQ- |
|---|---|---|---|---|---|
| plants | Genotype | Phenotype | 0018839 | 0018902 | 0018903 |
| 35 plants | Inbred recurrent | Indeterminate | C/C | A/A | G/G |
| | line | | | | |
| 34 plants | BC3F6 | Determinate | A/A | G/G | C/C |
| # of | | | SQ- | SQ- | SQ- |
| plants | Genotype | Phenotype | 0018904 | 0018907 | 0018909 |
| 35 plants | Inbred recurrent | Indeterminate | A/A | C/C | G/G |
| | line | | | | |
| 34 plants | BC3F6 | Determinate | G/G | A/A | A/A |

Seeds of determinate C. moschata plants from the BC3F6 population harboring QTL in chromosome 15 flanked by 6 SNP markers presented in Table 1, which is referred as “PUV008” are deposited as NCIMB accession no. 43470 at the NCIMB.

### Example 4. Phenotypic Data of Population of Cucurbita moschata Having Semi-Determinate Growth Habit

Phenotypic data of semi-determinate plants (i.e. plants carrying the QTL at a heterozygous state) are collected to understand plant architecture differences between semi-determinate and indeterminate plants. In this example, the semi-determinate plants are obtained by crossing the BC3F6 plant and the recurrent parent inbred line such as HF1). The semi-determinate plants with QTL harboring the determinate growth habit at a heterozygous state were analyzed to match their genotypes with phenotypes (such as number and length of internodes, male/female flower positions, number and length of branches, termination status of branches, number of fruits and fruit positions and the like) in comparison to the recurrent inbred plants, and BC3F6 plants.

Table 10 shows that the genotypes of SNP markers and field rating (i.e. phenotypes) for the growth habit (determinant, semi-determinant, or indeterminate), which indeed support the markers as effective tools for breeding. The data was collected from 65 plots. Five SNP markers used for identify genotypes of the plants and their qualitative field rating was determined by their phenotypic growth habits. The interpreted genotypes from selected five SNP markers are presented to show expected genotype of the plants for field trials. The quantitative field rating is presented to show phenotypic growth habit. Det refers to ‘Determinate’; Se-Det refers to ‘Semi-Determinate”; and In-Det refers to Indeterminate.” Plot 3403 is for an ‘Indeterminate’ check variety as a control.

TABLE 10
Plot tests for Cucurbita moschata plants based on their genotypes and phenotypes

| | Interpreted | | | | | | |
|---|---|---|---|---|---|---|---|
| | Genotype | SNP markers Genotype | | | | | |
| | from SNP | SQ- | SQ- | SQ- | SQ- | SQ- | Field |
| Plot | markers | 0018902 | 0018904 | 0018907 | 0018909 | 0018839 | Rating |
| 3401 | Det | Det | Det | Det | Det | Det | Semi |
| 3402 | Det | Det | Det | Det | Det | Det | Det |
| 3403 | In-Det | In-Det | In-Det | In-Det | In-Det | In-Det | In-det |
| 3404 | Se-Det | Se-Det | Se-Det | Se-Det | Se-Det | Se-Det | Det |
| 3405 | Det | Det | Det | Det | Det | Det | Det |
| 3406 | Det | Det | Det | Det | Det | Det | In-det |
| 3407 | Det | Det | Det | Det | Det | Det | Det |
| 3408 | Det | Det | Det | Det | Det | Det | Det |
| 3409 | Det | Det | Det | Det | Det | Det | Det |
| 3410 | Det | Det | Det | Det | Det | Det | Det |
| 3412 | Det | Det | Det | Det | Det | Det | Det |
| 3413 | Det | Det | Det | Det | Det | Det | Det |
| 3414 | Det | Det | Det | Det | Det | Det | Se-Det |
| 3415 | N/A** | Se-Det | Se-Det | Se-Det | Det | Se-Det | Se-Det |
| 3416 | N/A** | Se-Det | Se-Det | Se-Det | Det | Se-Det | Se-Det |
| 3417 | Se-Det | Se-Det | Se-Det | Se-Det | Se-Det | Se-Det | Se-Det |
| 3418 | Det | Det | Det | Det | Det | Det | Det |
| 3419 | Se-Det | Se-Det | Se-Det | Se-Det | Se-Det | Se-Det | Det |
| 3420 | Se-Det | Se-Det | Se-Det | Se-Det | Se-Det | Se-Det | Det |
| 3421 | Se-Det | Se-Det | Se-Det | Se-Det | Se-Det | Se-Det | Det |
| 3422 | Se-Det | Se-Det | Se-Det | Se-Det | Se-Det | Se-Det | Det |
| 3423 | Se-Det | Se-Det | Se-Det | Se-Det | Se-Det | Se-Det | Det |
| 3424 | Se-Det | Se-Det | Se-Det | Se-Det | Se-Det | Se-Det | Det |
| 3425 | Det | Det | Det | Det | Det | Det | Det |
| 3426 | Se-Det | Se-Det | Se-Det | Se-Det | Se-Det | Se-Det | In-det |
| 3427 | Se-Det | Se-Det | Se-Det | Se-Det | Se-Det | Se-Det | Det |
| 3428 | Se-Det | Se-Det | Se-Det | Se-Det | Se-Det | Se-Det | Det |
| 3429 | Se-Det | Se-Det | Se-Det | Se-Det | Se-Det | Se-Det | Det |
| 3430 | N/A** | Se-Det | Se-Det | Se-Det | Det | Se-Det | Det |
| 3431 | N/A** | Se-Det | Se-Det | Se-Det | Det | MISSING | Det |
| 3432 | N/A** | Se-Det | Se-Det | MISSING | Se-Det | Se-Det | Mixed²* |
| 3433 | Se-Det | Se-Det | Se-Det | Se-Det | Se-Det | Se-Det | Mixed²* |
| 3434 | Det | Det | Det | Det | Det | Det | Det |
| 3435 | Det | Det | Det | Det | Det | Det | Det |
| 3436 | Det | Det | Det | Det | Det | Det | Det |
| 3437 | Det | Det | Det | Det | Det | Det | Mixed¹* |
| 3438 | Det | Det | Det | Det | Det | Det | Det |
| 3439 | Det | Det | Det | Det | Det | Det | Det |
| 3440 | Se-Det | Se-Det | Se-Det | Se-Det | Se-Det | Se-Det | Det |
| 3441 | Se-Det | Se-Det | Se-Det | Se-Det | Se-Det | Se-Det | Det |
| 3442 | Det | Det | Det | Det | Det | Det | Det |
| 3443 | Det | Det | Det | Det | Det | Det | Det |
| 3444 | Det | Det | Det | Det | Det | Det | Det |
| 3445 | Det | Det | Det | Det | Det | Det | Det |
| 3446 | Se-Det | Se-Det | Se-Det | Se-Det | Se-Det | Se-Det | Det |
| 3447 | Se-Det | Se-Det | Se-Det | Se-Det | Se-Det | Se-Det | Mixed²* |
| 3449 | Det | Det | Det | Det | Det | Det | Det |
| 3451 | Det | Det | Det | Det | Det | Det | Se-Det |
| 3453 | Det | Det | Det | Det | Det | Det | Se-Det |
| 3455 | Det | Det | Det | Det | Det | Det | Se-Det |
| 3457 | Det | Det | Det | Det | Det | Det | Det |
| 3459 | Det | Det | Det | Det | Det | Det | Det |
| 3461 | Det | Det | Det | Det | Det | Det | Det |
| 3463 | Det | Det | Det | Det | Det | Det | Det |
| 3465 | Det | Det | Det | Det | Det | Det | Det |
| 3467 | Det | Det | Det | Det | Det | Det | Det |
| 3472 | Det | Det | Det | Det | Det | Det | Se-Det |
| 3475 | Det | Det | Det | De | Det | Det | Det |
| 3476 | Se-Det | Se-Det | Se-Det | Se-Det | Se-Det | Se-Det | Mixed²* |
| 3477 | Se-Det | Se-Det | Se-Det | Se-Det | Se-Det | Se-Det | Mixed¹* |
| 3478 | Se-Det | Se-Det | Se-Det | Se-Det | Se-Det | Se-Det | Se-Det |
| 3479 | Se-Det | Se-Det | Se-Det | Se-Det | Se-Det | Se-Det | Det |
| 3480 | Se-Det | Se-Det | Se-Det | Se-Det | Se-Det | Se-Det | Mixed1* |
| 3481 | Se-Det | Se-Det | Se-Det | Se-Det | Se-Det | Se-Det | Det |
| 3482 | Se-Det | Se-Det | Se-Det | Se-Det | Se-Det | Se-Det | Det |

Mixed¹* - Determinate and semi-determinate traits were detected in the designated plot.
Mixed²* - Determinate, semi-determinate, and indeterminate traits were detected in the designated plot.
N/A** - Genotype is unable to be determined or interpreted based on the given SNP marker genotypes.

Inventors collected the data for 4 SNP markers with 3 allele combinations (D/D; D/I; I/I) in relation to vine length and determinate/indeterminate. D and I alleles of SNP markers are presented in Table 1. D/D signifies homozygous on Determinate (D) allele. D/I signifies heterozygous with 1:1 of D allele and I allele, which shows almost the determinate growth habit trait. I/I allele signifies homozygous on Indeterminate (I) allele. There were clear difference between the phenotypes that were consistently predicted by the genotypes of some of the markers.

TABLE 11
Vine length and Determinate trait based on SNP marker alleles.

| | | | | Average | |
|---|---|---|---|---|---|
| | | | | vine length | Determinate |
| SQ-0018902 | SQ-0018904 | SQ-0018909 | SQ-0018914 | (cm) | (Yes/No) |
| Determinate | Determinate | Determinate | Determinate | 61 | Yes |
| Heterozygous | Heterozygous | Heterozygous | Heterozygous | 94 | Yes |
| Indeterminate | Indeterminate | Indeterminate | Indeterminate | 424 | No |

### Example 5. Candidate Gene Determining Determinate Growth Habit in Cucurbita Plants

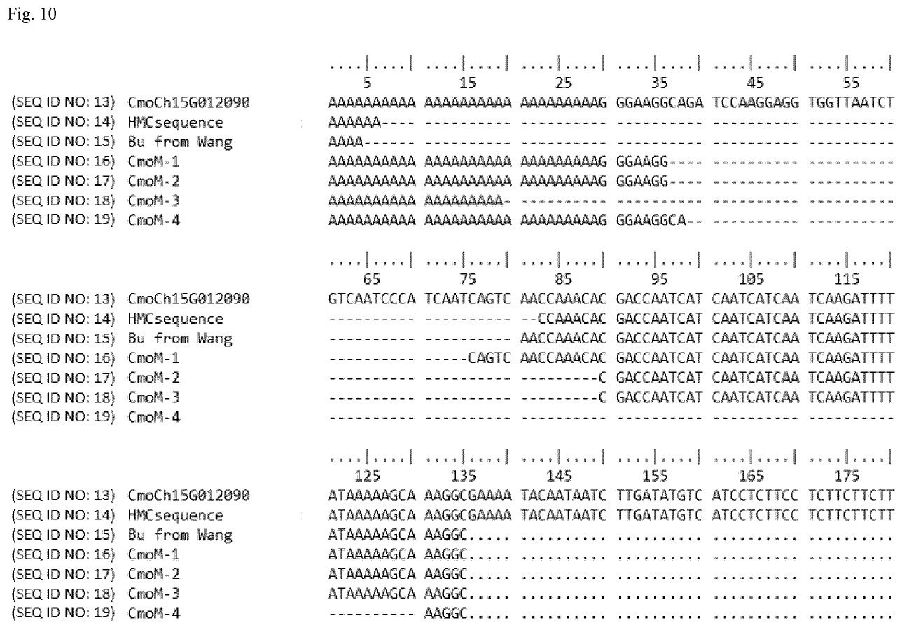

Further QTL and molecular analyses identified that CmoCh15G012090 is positioned between two SNP markers: SQ-0018902-N (SEQ ID NO: 7) and SQ-0018900 (SEQ ID NO: 5). CmoCh15G012090 is a transcription factor gene YABBY1 as a candidate gene giving rise to determinate phenotype of C. moschata plant. As presented in FIG. 10, HMC sequence obtained from the plants of the present disclosure (PUV008) shows a genetic modification with a portion of the 5′ UTR of CmoCh15G012090 gene deleted.

TABLE 12
Sequence Information

| Name | Sequence |
|---|---|
| 5′ UTR CmoCh15G012090 | AAAAAAAAAA AAAAAAAAAA AAAAAAAAAG GGAAGGCAGA |
| | TCCAAGGAGG TGGTTAATCT GTCAATCCCA TCAATCAGTC |
| | AACCAAACAC GACCAATCAT CAATCATCAA TCAAGATTTT |
| | ATAAAAAGCA AAGGCGAAAA TACAATAATC TTGATATGTC |
| | ATCCTCTTCC TCTTCTTCTT (SEQ ID NO: 13) |
| HMC sequence | AAAAAA CCAAACAC GACCAATCAT CAATCATCAA |
| | TCAAGATTTT ATAAAAAGCA AAGGCGAAAA TACAATAATC |
| | TTGATATGTC ATCCTCTTCC TCTTCTTCTT (SEQ ID NO: 14) |
| Bu from Wang | AAAA AACCAAACAC GACCAATCAT CAATCATCAA |
| | TCAAGATTTT ATAAAAAGCA AAGGCGAAAA TACAATAATC |
| | TTGATATGTC ATCCTCTTCC TCTTCTTCTT (SEQ ID NO: 15) |
| CmoM-1 | AAAAAAAAAA AAAAAAAAAA AAAAAAAAAG GGAAGG CAGTC |
| | AACCAAACAC GACCAATCAT CAATCATCAA TCAAGATTTT |
| | ATAAAAAGCA AAGGC (SEQ ID NO: 16) |
| CmoM-2 | AAAAAAAAAA AAAAAAAAAA AAAAAAAAAG GGAAGG C |
| | GACCAATCAT CAATCATCAA TCAAGATTTT ATAAAAAGCA |
| | AAGGC (SEQ ID NO: 17) |
| CmoM-3 | AAAAAAAAAA AAAAAAAAA C GACCAATCAT CAATCATCAA |
| | TCAAGATTTT ATAAAAAGCA AAGGC (SEQ ID NO: 18) |
| CmoM-4 | AAAAAAAAAA AAAAAAAAAA AAAAAAAAAG GGAAGGCA |
| | AAGGC (SEQ ID NO: 19) |

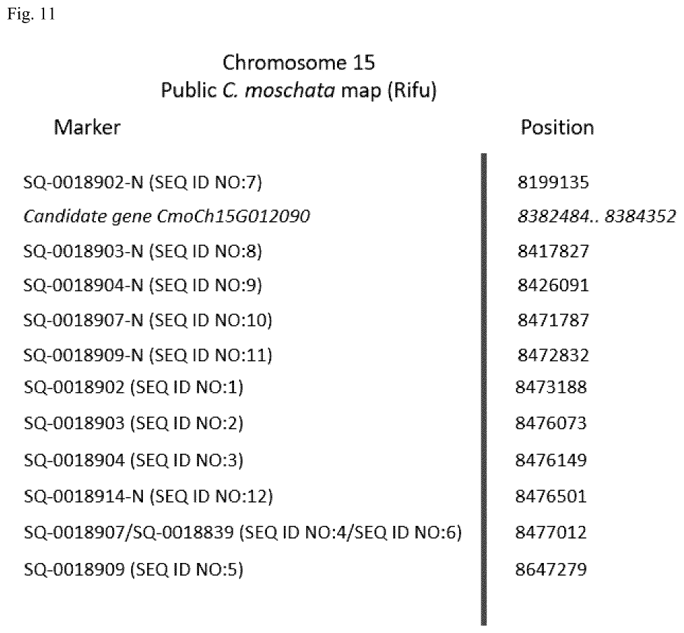

As further demonstrated in FIG. 11, SNP markers SQ-0018902-N (SEQ ID NO:7) and SQ-0018903-N (SEQ ID NO:8) are physically close to the candidate gene CmoCh15G012090. The 5′ UTR deletion of the gene in PUV008 plants indicates that bushy-like determinate growth habit with short internodes in PUV008 plants is caused by the genetic modification of this CmoCh15G012090 gene.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present disclosure is not entitled to antedate such publication by virtue of prior disclosure.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

### NUMBERED EMBODIMENTS OF THE DISCLOSURE

Notwithstanding the appended claims, the disclosure sets forth the following numbered embodiments:

1. A Cucurbita plant comprising a determinate growth habit, wherein said plant comprises a Quantitative Trait Locus (QTL) associated with a determinate growth habit on chromosome 15, wherein said QTL on chromosome 15 comprises Single Nucleotide Polymorphism (SNP) marker SQ-0018902-N (SEQ ID NO:7) on one side and SQ-0018839 (SEQ ID NO:6) on the other side; and wherein said QTL is present in the genome of Cucurbita moschata variety ‘PUV008,’ a representative sample of seed of which has been deposited under NCIMB No. 43740.
2. The Cucurbita plant according to embodiment 1, wherein said QTL further comprises a locus encompassing markers selected from the group consisting of SQ-0018903-N (SEQ ID NO:8), SQ-0018904-N (SEQ ID NO:9), SQ-0018907-N (SEQ ID NO:10), SQ-0018909-N (SEQ ID NO:11) and SQ-0018914-N (SEQ ID NO:12).
3. The Cucurbita plant according to embodiment 2, wherein said marker linked to said QTL is present in a homozygous or a heterozygous state for a Cucurbita plant having a determinate growth habit.
4. The Cucurbita plant according to embodiment 1, wherein said QTL is introgressed into a plant of genus Cucurbita having an indeterminate growth habit.
5. The Cucurbita plant according to embodiment 4, wherein the genus Cucurbita plant is a Cucurbita moschata (C. moschata), Cucurbita pepo (C. pepo), Cucurbita okeechobeensis (C. okeechobeensis), Cucurbita pedatifolia (C. pedatifolia), Cucurbita argyrosperma (C. argyrosperma) or Cucurbita maxima (C. maxima) plant.
6. The Cucurbita plant according to embodiment 1, wherein the Cucurbita plant displays a determinate growth habit with a limited number of short internodes when compared to a Cucurbita plant not having said QTL associated with a determinate growth habit.
7. The Cucurbita plant according to embodiment 1, wherein the determinate growth habit is conferred by a genetic modification or mutation at the 5′ UTR, exons, introns, or 3′ UTR of CmoCh15G012090.
8. The Cucurbita plant according to embodiment 1 or 7, wherein a portion of the 5′ UTR of CmoCh15G012090 is deleted.
9. The Cucurbita plant according to any one of embodiments 1 to 8, wherein said plant is a progeny of said Cucurbita moschata plant deposited with NCIMB No. 43740.
10. A cell of the Cucurbita plant according to any one of embodiments 1 to 8.
11. A plant part of the Cucurbita plant according to any one of embodiments 1 to 8.
12. A fruit of the Cucurbita plant according to any one of embodiments 1 to 8.
13. A seed of the Cucurbita plant according to any one of embodiments 1 to 8.
14. A method for producing a Cucurbita spp. plant with a determinate growth habit, the method comprising:
(i) crossing a first Cucurbita spp. plant having a determinant growth habit with a second Cucurbita spp. plant to produce progeny plants,
(ii) selecting one or more progeny plants having a determinate growth habit,
(iii) backcrossing the selected progeny plant having said determinate growth habit with the second Cucurbita spp. plant to produce backcross progeny plants,
(iv) selecting one or more backcross progeny plants having a determinate growth habit, and
(v) repeating steps (iii) and (iv) to produce selected second or higher backcross progeny plants having said determinate growth habit,
wherein the progeny plants selected in step (ii) and (iv) are selected by detecting the presence of one or more of SNP markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO: 12), SQ-0018839 (SEQ ID NO:6), and combinations thereof.
   15. The method according to embodiment 14, wherein said first or second Cucurbita plant is Cucurbita moschata.
   16. The method according to embodiment 14, wherein said first or second Cucurbita plant is a Cucurbita pepo (C. pepo), Cucurbita okeechobeensis (C. okeechobeensis), Cucurbita pedatifolia (C. pedatifolia), Cucurbita argyrosperma (C. argyrosperma) or Cucurbita maxima (C. maxima) plant.
   17. The method according to embodiment 15, wherein said C. moschata plant is C. moschata butternut type, C. moschata long neck butternut type, C. moschata flat round type, C. moschata round to oblate type, or hybrid thereof.
   18. A Cucurbita plant, a plant part thereof, or a plant cell thereof produced by the method according to any one of embodiments 14 to 17, wherein said plant has a determinate growth habit.
   19. A fruit of the Cucurbita plant according to embodiment 18
   20. A seed of the Cucurbita plant according to embodiment 18.
   21. A method for identifying a Cucurbita plant comprising a QTL associated with a determinate growth habit, the method comprising:
(i) providing a population of cultivated Cucurbita plants;
(ii) screening said population using a molecular marker assay which detects at least one SNP marker selected from the group consisting of: SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), 0018914-N (SEQ ID NO: 12), SQ-0018839 (SEQ ID NO:6) and combinations thereof; and
(iii) identifying and/or selecting a plant comprising at least one of the SNP markers of step (ii).
   22. The method according to embodiment 21, wherein said marker linked to said QTL is present in a homozygous or heterozygous state for a Cucurbita plant with a determinate growth habit.
   23. A method for producing a squash plant with a determinate or semi-determinate growth habit, the method comprising:
(i) crossing a first squash plant with a second squash plant to produce progeny plants,
(ii) selecting one or more progeny plants having a determinate or semi-determinate growth habit,
(iii) backcrossing the selected progeny plant having said determinate or semi-determinate growth habit with the second squash plant to produce backcross progeny plants,
(iv) selecting for backcross progeny plants having a determinate or semi-determinate growth habit, and
(v) repeating steps (iii) and (iv) to produce selected second or higher backcross progeny plants having a determinate or semi-determinate growth habit,
wherein the progeny plants selected in step (ii) and (iv) are selected by detecting the presence of one or more of markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO: 12), and SQ-0018839 (SEQ ID NO:6).
   24. The method according to embodiment 23, wherein said first squash plant comprises a determinate growth habit and said second squash plant comprises an indeterminate growth habit.
   25. The method according to any one of embodiments 23 to 24, wherein said first or second squash plant is a Cucurbita moschata (C. moschata), Cucurbita pepo (C. pepo), Cucurbita okeechobeensis (C. okeechobeensis), Cucurbita ficifolia (C. ficifolia), Cucurbita argyrosperma (C. argyrosperma) or a Cucurbita maxima (C. maxima) plant.
   26. The method according to embodiment 25, wherein said C. moschata plant is C. moschata butternut type, C. moschata long neck butternut type, C. moschata flat round type, C. moschata round to oblate type, or hybrid thereof.
   27. A selected squash plant, a plant part thereof, or a plant cell thereof produced by the method according to any one of embodiments 23 to 26, wherein said plant has a determinate growth habit.
   28. A fruit of the squash plant according to embodiment 27.
   29. A seed of the squash plant according to embodiment 27.
   30. A seed, plant, plant part, or plant cell of a Cucurbita moschata plant variety designated ‘PUV008,’ wherein a representative sample of seed of said plant has been deposited under NCIMB No. 43740.
   31. A Cucurbita moschata plant, a part thereof, or a cell thereof, produced by growing the seed of embodiment 30, wherein the Cucurbita moschata plant has all of the physiological and morphological characteristics of Cucurbita moschata designated ‘PUV008’ when grown under the same environmental conditions.
   32. The Cucurbita moschata plant, the part thereof, or the cell thereof of embodiment 31, wherein the plant part is selected from the group consisting of a leaf, a flower, a fruit, a stalk, a root, a rootstock, a scion, a meristem, and a cell.
   33. A tissue culture of regenerable cells produced from the Cucurbita moschata plant or the part thereof of embodiment 31, wherein a Cucurbita moschata plant regenerated from the tissue culture has all of the physiological and morphological characteristics of Cucurbita moschata designated ‘PUV008’ when grown under the same environmental conditions.
   34. A Cucurbita moschata plant regenerated from the tissue culture of embodiment 33.
   35. A method for harvesting a Cucurbita moschata fruit, the method comprising: (a) growing the Cucurbita moschata plant of embodiment 31 to produce a Cucurbita moschata fruit, and (b) harvesting said Cucurbita moschata fruit.
   36. A method for producing a Cucurbita moschata seed, the method comprising: (a) crossing a first Cucurbita moschata plant with a second Cucurbita moschata plant and (b) harvesting the resultant Cucurbita moschata seed, wherein said first Cucurbita moschata plant and/or second Cucurbita moschata plant is the Cucurbita moschata plant of embodiment 31.
   37. A method for producing a Cucurbita moschata seed, the method comprising: (a) self-pollinating the Cucurbita moschata plant of embodiment 31 and (b) harvesting the resultant Cucurbita moschata seed.
   38. A method of vegetatively propagating the Cucurbita moschata plant of embodiment 31, the method comprising: (a) collecting a part capable of being propagated from the plant of embodiment 31 and (b) regenerating a plant from said part.
   39. The method of embodiment 38, further comprising (c) harvesting a fruit from said regenerated plant.
   40. A plant produced by the method of embodiment 38, wherein said plant has all of the physiological and morphological characteristics of Cucurbita moschata designated ‘PUV008’ deposited under NCIMB No. 43740.
   41. A Cucurbita moschata fruit selected from the group consisting of: (a) a Cucurbita moschata fruit produced from the plant of embodiment 31; (b) a Cucurbita moschata fruit produced by the method of embodiment 35, and (c) a Cucurbita moschata fruit produced from the method of embodiment 39.
   42. A method of producing a Cucurbita moschata from Cucurbita moschata designated ‘PUV008’, the method comprising: (a) self-pollinating the Cucurbita moschata plant of embodiment 31 at least once to produce a progeny Cucurbita moschata plant obtained from Cucurbita moschata designated ‘PUV008’.
   43. The method of embodiment 42, further comprising the steps of:
(b) crossing the progeny Cucurbita moschata plant obtained from the Cucurbita moschata designated ‘PUV008’ with itself or a second Cucurbita moschata plant to produce a progeny seed of a subsequent generation;
(c) growing a progeny plant from the progeny seed of the subsequent generation;
(d) crossing the progeny plant of the subsequent generation with itself or a second Cucurbita moschata plant to produce a Cucurbita moschata plant derived from the Cucurbita moschata designated ‘PUV008’; and
(e) repeating step (c) and (d) for at least one generation to produce a Cucurbita moschata plant further derived from the Cucurbita moschata designated ‘PUV008’.
   44. A method of producing a Cucurbita moschata plant obtained from Cucurbita moschata designated ‘PUV008’, the method comprising: (a) crossing the Cucurbita moschata plant of embodiment 31 with a second Cucurbita moschata plant to produce a progeny Cucurbita moschata plant obtained from Cucurbita moschata designated ‘PUV008’.
   45. The method of embodiment 44, further comprising the steps of:
(b) crossing the progeny Cucurbita moschata plant obtained from the Cucurbita moschata plant designated ‘PUV008’ with itself or a second Cucurbita moschata plant to produce a progeny seed of a subsequent generation;
(c) growing a progeny plant from the progeny seed of the subsequent generation;
(d) crossing the progeny plant of the subsequent generation with itself or a second Cucurbita moschata plant to produce a Cucurbita moschata plant derived from the Cucurbita moschata plant designated ‘PUV008’; and
(e) repeating step (c) and (d) for at least one generation to produce a Cucurbita moschata plant further derived from the Cucurbita moschata plant designated ‘PUV008’.
   46. A method of producing a plant of Cucurbita moschata designated ‘PUV008’ comprising at least one desired trait, the method comprising introducing a single locus conversion conferring the desired trait into Cucurbita moschata designated ‘PUV008’, whereby a plant of Cucurbita moschata designated ‘PUV008’ comprising the desired trait is produced.
   47. A Cucurbita moschata plant, a part thereof, or a cell thereof, produced by the method of embodiment 46, wherein the plant, the part, or the cell thereof comprises a single locus conversion and otherwise all of the physiological and morphological characteristics of Cucurbita moschata ‘PUV008’ deposited under NCIMB No. 43740.
   48. The plant of embodiment 47, wherein the single locus conversion confers said plant with male sterility, male fertility, herbicide resistance, insect resistance, disease resistance, water stress tolerance, heat tolerance, improved standability, enhanced plant vigor, improved shelf life, delayed senescence or controlled ripening, and increased nutritional quality.
   49. The plant of embodiment 47, wherein the single locus conversion is an artificially mutated gene or nucleotide sequence.
   50. The plant of embodiment 47, wherein the single locus conversion is introduced into the plant by a genetic transformation or a gene editing technique with a nuclease selected from the group consisting of Zinc finger nuclease (ZFN), Transcription Activation-Like Effector Nuclease (TALEN), Clustered Regularly Interspaced Short Palindromic Repeats-associated Cas endonuclease (CRISPR-Cas), meganuclease, homing endonuclease, and RNA-guided nuclease.
   51. A method of producing a Cucurbita moschata plant, the method comprising: grafting a rootstock or a scion of the Cucurbita moschata plant of embodiment 31 to another Cucurbita moschata plant.
   52. A method for producing nucleic acids, the method comprising: isolating nucleic acids from the plant of embodiment 31, a part, or a cell thereof.
   53. A method for producing a second Cucurbita moschata plant, the method comprising applying plant breeding techniques to the plant or part of embodiment 31 to produce the second Cucurbita moschata plant.
   54. A method of producing a commodity plant product, the method comprising obtaining the plant of embodiment 31 or a part thereof and producing said commodity plant product therefrom.
   55. A Cucurbita spp. seed, plant, part thereof, or cell thereof, whose genome contains at least one copy of a dominant allele for a determinate or semi-determinate growth habit, wherein the allele localizes to chromosome 15 and is flanked on either side by one or more SNP markers selected from the group consisting of SQ-0018902-N (SEQ ID NO:7), SQ-0018903-N (SEQ ID NO:8), SQ-0018904-N (SEQ ID NO:9), SQ-0018907-N (SEQ ID NO:10), SQ-0018909-N (SEQ ID NO:11), SQ-0018914-N (SEQ ID NO: 12), SQ-0018839 (SEQ ID NO:6), and combinations thereof.
   56. The Cucurbita spp. seed, plant, part thereof, or cell thereof, of embodiment 55, wherein the dominant allele for a determinate or semi-determinate growth habit on chromosome 15 comprises a deleted portion of the 5′ UTR of CmoCh15G012090.

Another set of the numbered embodiments are provided below for the deposited Cucurbita variety PUV008 and methods of producing the variety and its progenies.

1. A plant of Cucurbita variety PUV008, having a determinate growth habit, wherein a representative sample of plant tissue of said variety was deposited under NCIMB No. 43740.
2. A Cucurbita seed produced by growing the plant of embodiment 1.
3. A method of producing a Cucurbita plant, or part thereof, produced by growing the seed of embodiment 2.
4. A Cucurbita plant, or part thereof, having all of the physiological and morphological characteristics of the Cucurbita plant of embodiment 1.
5. A method of vegetatively propagating the plant of embodiment 1, comprising the steps of:
a) collecting tissue or cells capable of being propagated from said plant;
b) cultivating said tissue or cells to obtain proliferated shoots or plantlets; and
c) rooting said proliferated shoots or plantlets to obtain rooted shoots or rooted plantlets.
   6. A Cucurbita plant produced by growing the rooted shoots or rooted plantlets of embodiment 5.
   7. A method of determining the genotype of the Cucurbita plant of embodiment 1, wherein said method comprises obtaining a sample of nucleic acids from said plant and detecting in said nucleic acids a plurality of polymorphisms.
   8. A method of introducing a mutation into the genome of a PUV008 plant, said method comprising mutagenesis of the plant, or plant part thereof, of embodiment 1, wherein said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.
   9. A method of genetically modifying the plant of embodiment 1, wherein said genetic modification is selected from agrobacterium-mediated gene transfer, protoplast transformation, or biolistic transformation.
   10. A plant produced by the method of embodiment 9, wherein said plant has the determinate growth habit.
   11. A method of editing the genome of Cucurbita plant PUV008, said method comprising editing the genome of the plant, or a part thereof, of embodiment 1, wherein said method is selected from the group comprising zinc finger nucleases, transcription activator-like effector nucleases (TALENs), engineered homing endonucleases/meganucleases, and the clustered regularly interspaced short palindromic repeat (CRISPR)-associated protein9 (Cas9) system.
   12. A Cucurbita plant produced by the method of embodiment 11.
   13. A plant, or a plant part thereof, produced by growing a plant of Cucurbita variety PUV008, wherein a representative sample of plant tissue of said variety was deposited under NCIMB No.
   43740, and wherein the plant or plant part comprises at least one cell of Cucurbita variety PUV008.
   14. A tissue or cell culture of regenerable cells produced from the plant or plant part of embodiment 13.
   15. A Cucurbita plant regenerated from the tissue or cell culture of embodiment 14, wherein said plant has all of the morphological and physiological characteristics of Cucurbita variety PUV008.
   16. A method for producing a seed or an embryo, wherein the method comprises crossing Cucurbita variety PUV008, wherein a representative sample of plant tissue of said variety was deposited under NCIMB No. 43740, with a different plant and harvesting the resultant seed or embryo.
   17. A method for producing a seed or an embryo, wherein the method comprises selfing Cucurbita variety PUV008, wherein a representative sample of plant tissue of said variety was deposited under NCIMB No. 43740, and harvesting the resultant seed or embryo.
   18. A plant produced by growing the seed or embryo produced by the method of embodiment 17, wherein said plant has the determinate growth habit.
   19. A method for developing a Cucurbita plant having a determinate growth habit, wherein said method comprises applying plant breeding techniques to the plant of Cucurbita variety PUV008, having a determinate growth habit, wherein a representative sample of plant tissue of said variety was deposited under NCIMB No. 43740, to produce a plant having a determinate growth habit.
   20. The method of embodiment 19, wherein said plant breeding technique is recurrent selection, mass selection, hybridization, open-pollination, backcrossing, or pedigree breeding.
   21. The method of embodiment 19, wherein said plant breeding technique is mutation breeding, and wherein said mutation selected is spontaneous or artificially induced.
   22. The method of embodiment 19, wherein said plant breeding technique is marker enhanced selection.
   23. A method for producing a Cucurbita plant having a determinate growth habit, said method comprising:
a) crossing two Cucurbita plants,
b) harvesting the resultant Cucurbita seed,
c) growing the seed from step b) to produce progeny, and
d) selecting resulting progeny for the determinate growth habit; wherein at least one of the two Cucurbita plants is ‘PUV008,’ representative tissue of ‘PUV008’ having been deposited under NCIMB No. 43740.
   24. The method of embodiment 23, further comprising: e) crossing the selected progeny plant having the determinate growth habit another Cucurbita plant, or with itself, and selecting resulting subsequent progeny for the determinate growth habit.
   25. The method of embodiment 23, further comprising: e) propagating the selected progeny plant via seed, cutting, or tissue culture.
   26. A method for producing a Cucurbita plant having a determinate growth habit, said method comprising: propagating a vegetative cutting from a stock Cucurbita plant, thereby producing the Cucurbita plant having a determinate growth habit; wherein the stock Cucurbita plant has the determinate growth habit and is a product of a single cross between: a parental line plant with another plant, or with itself; wherein the parental line plant is ‘PUV008,’ representative tissue of ‘PUV008’ having been deposited under NCIMB No. 43740.
   27. A method for producing a Cucurbita plant having a determinate growth habit, said method comprising: propagating a vegetative cutting from a stock Cucurbita plant, thereby producing the Cucurbita plant having a determinate growth habit, wherein the stock Cucurbita plant has the a determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740.
   28. The method of embodiment 27, wherein said plant breeding technique is recurrent selection, mass selection, hybridization, open-pollination, backcrossing, or pedigree breeding.
   29. A method for producing a seed that develops into a Cucurbita plant having a determinate growth habit, said method comprising: crossing a first Cucurbita plant with a stock Cucurbita plant and harvesting resulting seed, thereby producing the seed that develops into the Cucurbita plant having the determinate growth habit; wherein the first Cucurbita plant and/or the stock Cucurbita plant has the determinate growth habit and is a product of one or more crosses comprising a cross between: a parental line plant with another Cucurbita plant, or with itself; wherein the parental line plant is ‘PUV008,’ representative tissue of ‘PUV008’ having been deposited under NCIMB No. 43740.
   30. A method for producing a seed that develops into a Cucurbita plant having a determinate growth habit, said method comprising: crossing a first Cucurbita plant with a stock Cucurbita plant, thereby producing the seed that develops into the Cucurbita plant having the determinate growth habit; wherein the first Cucurbita and/or the stock Cucurbita plant has the determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740.
   31. The method of embodiment 30, wherein said plant breeding technique is recurrent selection, mass selection, hybridization, open-pollination, backcrossing, or pedigree breeding.
   32. The method of embodiment 30, wherein said plant breeding technique is mutation breeding, and wherein said mutation breeding selects for a mutation that is spontaneous or artificially induced.
   33. The method of embodiment 30, wherein said plant breeding technique is marker enhanced selection.
   34. A method for producing a seed that develops into a Cucurbita plant having a determinate growth habit, said method comprising: selfing a stock Cucurbita plant, thereby producing the seed that develops into the Cucurbita plant having the determinate growth habit; wherein the stock Cucurbita plant has determinate growth habit and is a product of applying a plant breeding technique to a parental line plant having the spotted flower color pattern; wherein the parental line plant is ‘PUV008,’ representative tissue of ‘PUV008’ having been deposited under NCIMB No. 43740.
   35. The method of embodiment 34, wherein said plant breeding technique is recurrent selection, mass selection, hybridization, open-pollination, backcrossing, or pedigree breeding.
   36. The method of embodiment 34, wherein said plant breeding technique is mutation breeding, and wherein said mutation breeding selects for a mutation that is spontaneous or artificially induced.
   37. The method of embodiment 34, wherein said plant breeding technique is marker enhanced selection.
   38. A method for producing a Cucurbita plant having a determinate growth habit, said method comprising: propagating a vegetative cutting from a stock Cucurbita plant, thereby producing the Cucurbita plant having the determinate growth habit; wherein the stock Cucurbita plant has the determinate growth habit and is a product of one or more crosses comprising a cross between: a parental line plant with another Cucurbita plant, or with itself; wherein the parental line plant is ‘PUV008,’ representative tissue of ‘PUV008’ having been deposited under NCIMB No. 43740.
   39. A method for producing a Cucurbita plant having a determinate growth habit, said method comprising: propagating a vegetative cutting from a stock Cucurbita plant, thereby producing the Cucurbita plant having the determinate growth habit; wherein the stock Cucurbita plant has the determinate growth habit and is a product of applying a plant breeding technique to a parental line which is ‘PUV008,’ representative tissue of ‘PUV008’ having been deposited under NCIMB No. 43740.
   40. The method of embodiment 39, wherein said plant breeding technique is selected from the group consisting of: recurrent selection, mass selection, hybridization, open-pollination, backcrossing, pedigree breeding, and marker enhanced selection.
   41. The method of embodiment 39, wherein said plant breeding technique is mutation breeding, and wherein said mutation breeding selects for a mutation that is spontaneous or artificially induced.
   42. A method for producing a Cucurbita plant having a determinate growth habit, said method comprising: rooting a vegetative cutting from a stock Cucurbita plant, thereby producing the Cucurbita plant having the determinate growth habit; wherein the stock Cucurbita plant has the determinate growth habit and is a product of one or more crosses comprising a cross between: a parental line plant with another Cucurbita plant, or with itself; wherein the parental line plant is ‘PUV008,’ representative tissue of ‘PUV008’ having been deposited under NCIMB No.
   43740.
   43. A method for producing a Cucurbita plant having a determinate growth habit, said method comprising: rooting a vegetative cutting from a stock Cucurbita plant, thereby producing the Cucurbita plant having the determinate growth habit; wherein the stock Cucurbita plant has the determinate growth habit and is a product of applying a plant breeding technique to a parental line which is ‘PUV008,’ representative tissue of ‘PUV008’ having been deposited under NCIMB No. 43740.
   44. The method of embodiment 43, wherein said plant breeding technique is selected from the group consisting of: recurrent selection, mass selection, hybridization, open-pollination, backcrossing, pedigree breeding, and marker enhanced selection.
   45. The method of embodiment 43, wherein said plant breeding technique is mutation breeding, and wherein said mutation breeding selects for a mutation that is spontaneous or artificially induced.
   46. A method for producing a Cucurbita plant having a determinate growth habit, said method comprising: regenerating tissue from a stock Cucurbita plant via tissue culture, thereby producing the Cucurbita plant having the determinate growth habit; wherein the stock Cucurbita plant has the determinate growth habit and is a product of one or more crosses comprising a cross between: a parental line plant with another Cucurbita plant, or with itself; wherein the parental line plant is ‘PUV008,’ representative tissue of ‘PUV008’ having been deposited under NCIMB No. 43740.
   47. A method for producing a Cucurbita plant having a determinate growth habit, said method comprising: regenerating tissue from a stock Cucurbita plant via tissue culture, thereby producing the Cucurbita plant having the determinate growth habit; wherein the stock Cucurbita plant has the determinate growth habit and is a product of applying a plant breeding technique to a parental line which is ‘PUV008,’ representative tissue of ‘PUV008’ having been deposited under NCIMB No. 43740.
   48. The method of embodiment 47, wherein said plant breeding technique is selected from the group consisting of: recurrent selection, mass selection, hybridization, open-pollination, backcrossing, pedigree breeding, and marker enhanced selection.
   49. The method of embodiment 47, wherein said plant breeding technique is mutation breeding, and wherein said mutation breeding selects for a mutation that is spontaneous or artificially induced.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### REFERENCES

Taylor, M. J. and Brant, J. 2002. Trends in world cucurbit production, 1991 to 2001. In: Cucurbitaceae 2002, pp. 373-279, Maynard, D. N., Ed., ASHS Press, Alexandria, VA.
Loy, J. Brent (2004): Morpho-Physiological Aspects of Productivity and Quality in Squash and Pumpkin (Cucurbita spp.), Critical Reviews in Plant Sciences, 23:4, 337-363
Pujol, M., et al. (2019). Mapping Cucumber vein yellowing virus resistance in cucumber (Cucumis sativus L.) by using BSA-seq analysis. Frontiers in plant science, 10, 1583.
Ramos, A., Fu, Y., Michael, V., & Meru, G. (2020). QTL-seq for identification of loci associated with resistance to Phytophthora crown rot in squash. Scientific reports, 10(1), 1-8.
Zhang, J., & Panthee, D. R. (2019). PyBSASeq: a novel, simple, and effective algorithm for BSA-Seq data analysis. BioRxiv, 654137.
Zou, C., Wang, P., & Xu, Y. (2016). Bulked sample analysis in genetics, genomics and crop improvement. Plant biotechnology journal, 14(10), 1941-1955.
Song, J., Li, Z., Liu, Z., Guo, Y., & Qiu, L. J. (2017). Next-generation sequencing from bulked-segregant analysis accelerates the simultaneous identification of two qualitative genes in soybean. Frontiers in plant science, 8, 919.
Yoshitsu, Y., Takakusagi, M., Abe, A., Takagi, H., Uemura, A., Yaegashi, H., . . . & Yokoi, S. (2017). QTL-seq analysis identifies two genomic regions determining the heading date of foxtail millet, Setaria italica (L.) P. Beauv. Breeding science, 67(5), 518-527.
Sun, J., Yang, L., Wang, J., Liu, H., Zheng, H., Xie, D., . . . & Zou, D. (2018). Identification of a cold-tolerant locus in rice (Oryza sativa L.) using bulked segregant analysis with a next-generation sequencing strategy. Rice, 11(1), 24.
Pang, W., Fu, P., Li, X., Zhan, Z., Yu, S., & Piao, Z. (2018). Identification and mapping of the Clubroot resistance gene CRd in Chinese cabbage (Brassica rapa ssp. pekinensis). Frontiers in plant science, 9, 653.
Deokar, A., Sagi, M., Daba, K., & Tar'an, B. (2019). QTL sequencing strategy to map genomic regions associated with resistance to ascochyta blight in chickpea. Plant biotechnology journal, 17(1), 275-288.
Zhang, X., Wang, G., Chen, B., Du, H., Zhang, F., Zhang, H., . . . & Geng, S. (2018). Candidate genes for first flower node identified in pepper using combined SLAF-seq and BSA. PLoS One, 13(3), e0194071.
Wang, H., Cheng, H., Wang, W., Liu, J., Hao, M., Mei, D., . . . & Hu, Q. (2016). Identification of BnaYUCCA6 as a candidate gene for branch angle in Brassica napus by QTL-seq. Scientific reports, 6, 38493.
Wen, C., Zhao, W., Liu, W., Yang, L., Wang, Y., Liu, X., . . . & Li, J. (2019). CsTFL1 inhibits determinate growth and terminal flower formation through interaction with CsNOT2a in cucumber. Development, 146(14), dev180166.
Gebremeskel, H., Dou, J., Li, B., Zhao, S., Muhammad, U., Lu, X., . . . & Liu, W. (2020). Molecular Mapping and Candidate Gene Analysis for GA3 Responsive Short Internode in Watermelon (Citrullus lanatus). International journal of molecular sciences, 21(1), 290.
Zhao, W., Gu, R., Che, G., Cheng, Z., & Zhang, X. (2018). CsTFL1b may regulate the flowering time and inflorescence architecture in cucumber (Cucumis sativus L.). Biochemical and biophysical research communications, 499(2), 307-313.
Zhang, H., & Zhang, Y. (2020). Molecular cloning and functional characterization of CmFT (FLOWERING LOCUS T) from Cucumis melo L. Journal of Genetics, 99(1).
Njogu, M. K., Yang, F., Li, J., Wang, X., Ogweno, J. O., & Chen, J. (2020). A novel mutation in TFL1 homolog sustaining determinate growth in cucumber (Cucumis sativus L.). Theoretical and Applied Genetics, 1-10.
Zhao, W., Gu, R., Che, G., Cheng, Z., & Zhang, X. (2018). CsTFL1b may regulate the flowering time and inflorescence architecture in cucumber (Cucumis sativus L.). Biochemical and biophysical research communications, 499(2), 307-313.
Dong, W., Wu, D., Li, G., Wu, D., & Wang, Z. (2018). Next-generation sequencing from bulked segregant analysis identifies a dwarfism gene in watermelon. Scientific reports, 8(1), 1-7.
Zhang, T., Liu, J., Liu, S., Ding, Z., Luan, F., & Gao, P. (2019). Bulked-segregant analysis identified a putative region related to short internode length in melon. HortScience, 54(8), 1293-1298.
Wei, C., Zhu, C., Yang, L., Zhao, W., Ma, R., Li, H., . . . & Zhang, X. (2019). A point mutation resulting in a 13 bp deletion in the coding sequence of Cldf leads to a GA-deficient dwarf phenotype in watermelon. Horticulture research, 6(1), 1-12.
Yang, L., Zhu, H., Zhang, M., Sun, S., Yang, S., Li, J., . . . & Liu, D. (2019). A single nucleotide deletion in the ABC transporter gene leads to a dwarf phenotype in watermelon. Frontiers in plant science, 10, 1399.
Hou, S., Niu, H., Tao, Q., Wang, S., Gong, Z., Li, S., . . . & Li, Z. (2017). A mutant in the CsDET2 gene leads to a systemic brassinosteriod deficiency and super compact phenotype in cucumber (Cucumis sativus L.). Theoretical and applied genetics, 130(8), 1693-1703.
Xu, L., Wang, C., Cao, W., Zhou, S., & Wu, T. (2018). CLAVATA1-type receptor-like kinase CsCLAVATA1 is a putative candidate gene for dwarf mutation in cucumber. Molecular Genetics and Genomics, 293(6), 1393-1405.
DePristo, M., Banks, E., Poplin, R. et al. A framework for variation discovery and genotyping using next-generation DNA sequencing data. Nat Genet 43, 491-498 (2011).
Van der Auwera G A, Carneiro M O, Hartd C, Poplin R, Del Angel G, Levy-Moonshine A, Jordan T, Shakir K, Roazen D, Thibault J, Banks E, Garimella K V, Altshuler D, Gabriel S,
DePristo M A. From FastQ data to high confidence variant calls: the Genome Analysis Toolkit best practices pipeline. Curr Protoc Bioinformatics. 2013; 43(1110):11.10.1-11.10.33.
R Development Core Team (2008) R: A Language and Environment for Statistical Computing. R Foundation for Statistical Computing, Vienna, Austria: ISBN 3-900051-07-0, http://www.R-project.org.
Mansfeld B N, Grumet R. QTLsegr: An R Package for Bulk Segregant Analysis with Next-Generation Sequencing. Plant Genome. 2018 July; 11(2) doi:10.3835/plantgenome2018.01.0006
Wang S, Wang K, Li Z, Li Y, He J, Li H, Wang B, Xin T, Tian H, Tian J, Zhang G, Li H, Huang S, Yang X. Architecture design of cucurbit crops for enhanced productivity by a natural allele. Nat Plants. 2022 December; 8(12):1394-1407.

1. A plant of Cucurbita variety PUV008, having a determinate growth habit, wherein a representative sample of plant tissue of said variety was deposited under NCIMB No. 43740.

2. A Cucurbita seed produced by growing the plant of claim 1.

3. A method of producing a Cucurbita plant, or part thereof, produced by growing the seed of claim 2.

4. A Cucurbita plant, or part thereof, having all of the physiological and morphological characteristics of the Cucurbita plant of claim 1.

5. A method of vegetatively propagating the plant of claim 1, comprising the steps of:
a) collecting tissue or cells capable of being propagated from said plant;
b) cultivating said tissue or cells to obtain proliferated shoots or plantlets; and
c) rooting said proliferated shoots or plantlets to obtain rooted shoots or rooted plantlets.

6. A Cucurbita plant produced by growing the rooted shoots or rooted plantlets of claim 5.

7. A method of determining the genotype of the Cucurbita plant of claim 1, wherein said method comprises obtaining a sample of nucleic acids from said plant and detecting in said nucleic acids a plurality of polymorphisms.

8. A method of introducing a mutation into the genome of a PUV008 plant, said method comprising mutagenesis of the plant, or plant part thereof, of claim 1, wherein said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.

9. A method of genetically modifying the plant of claim 1, wherein said genetic modification is selected from agrobacterium-mediated gene transfer, protoplast transformation, or biolistic transformation.

10. A plant produced by the method of claim 9, wherein said plant has the determinate growth habit.

11. A method of editing the genome of Cucurbita plant PUV008, said method comprising editing the genome of the plant, or a part thereof, of claim 1, wherein said method is selected from the group comprising zinc finger nucleases, transcription activator-like effector nucleases (TALENs), engineered homing endonucleases/meganucleases, and the clustered regularly interspaced short palindromic repeat (CRISPR)-associated protein9 (Cas9) system.

12. A Cucurbita plant produced by the method of claim 11.

13. A method for developing a Cucurbita plant having a determinate growth habit, wherein said method comprises applying plant breeding techniques to the plant of claim 1.

14. The method of claim 13, wherein said plant breeding technique is recurrent selection, mass selection, hybridization, open-pollination, backcrossing, or pedigree breeding.

15. The method of claim 13, wherein said plant breeding technique is mutation breeding, and wherein said mutation selected is spontaneous or artificially induced.

16. The method of claim 13, wherein said plant breeding technique is marker enhanced selection.

17. A method for producing a Cucurbita plant having a determinate growth habit, said method comprising:
a) crossing two Cucurbita plants,
b) harvesting the resultant Cucurbita seed,
c) growing the seed from step b) to produce progeny, and
d) selecting resulting progeny for the determinate growth habit; wherein at least one of the two Cucurbita plants is the plant of claim 1.

18. The method of claim 17, further comprising: e) crossing the selected progeny plant having the determinate growth habit another Cucurbita plant, or with itself, and selecting resulting subsequent progeny for the determinate growth habit.

19. The method of claim 17, further comprising: e) propagating the selected progeny plant via seed, cutting, or tissue culture.

20. A method for producing a Cucurbita plant having a determinate growth habit, said method comprising: propagating a vegetative cutting from a stock Cucurbita plant, thereby producing the Cucurbita plant having a determinate growth habit, wherein the stock Cucurbita plant has the determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740.

21. The method of claim 20, wherein said plant breeding technique is recurrent selection, mass selection, hybridization, open-pollination, backcrossing, or pedigree breeding.

22. A method for producing a seed that develops into a Cucurbita plant having a determinate growth habit, said method comprising: crossing a first Cucurbita plant with a stock Cucurbita plant, thereby producing the seed that develops into the Cucurbita plant having the determinate growth habit; wherein the first Cucurbita and/or the stock Cucurbita plant has the determinate growth habit and is a product of applying a plant breeding technique to a parental line plant having the determinate growth habit; wherein the parental line plant is ‘PUV008,’ representative tissue of ‘PUV008’ having been deposited under NCIMB No. 43740.

23. The method of claim 22, wherein said plant breeding technique is recurrent selection, mass selection, hybridization, open-pollination, backcrossing, or pedigree breeding.

24. The method of claim 22, wherein said plant breeding technique is mutation breeding, and wherein said mutation breeding selects for a mutation that is spontaneous or artificially induced.

25. The method of claim 22, wherein said plant breeding technique is marker enhanced selection.

26. A method of determining the genotype of the Cucurbita plant of claim 1, wherein said method comprises obtaining a sample of nucleic acids from said plant and detecting in said nucleic acids a plurality of polymorphisms.

27. A method of introducing a mutation into the genome of a PUV008 plant, said method comprising mutagenesis of the plant, or plant part thereof, of claim 1, wherein said method of mutagenesis is selected from the group consisting of temperature, long term seed storage, somaclonal variation, radiation, chemical agents, targeting induced local lesions in genomes, site-directed mutagenesis, and genome editing, and wherein the resulting plant comprises at least one genome mutation.

28. A method of genetically modifying the plant of claim 1, wherein said genetic modification is selected from agrobacterium-mediated gene transfer, protoplast transformation, or biolistic transformation.

29. A plant produced by the method of claim 28, wherein said plant has the determinate growth habit.
